SEQUENCE LISTING (4 sequences)

SEQ ID NO: 1 - protein, 221 aa, Artificial Sequence
  Description of Artificial Sequence Synthetic polypeptide
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 227 aa, Artificial Sequence
  Description of Artificial Sequence Synthetic polypeptide
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, Artificial Sequence
  Description of Artificial Sequence Synthetic polypeptide
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - DNA, 12 bp, Homo sapiens
ctgattggccaa

(19) United States
(12) Patent Application Publication
(10) Pub. No.: US 2024/0228623 A1
(43) Pub. Date: Jul. 11, 2024

(54) METHODS FOR REDUCING MATERNAL AUTOANTIBODIES

(71) Applicant: MaraBio Systems, Inc., Menlo Park, CA (US)

(72) Inventor: James N. WOODY, Menlo Park, CA (US)

(21) Appl. No.: 18/042,195

(22) PCT Filed: Aug. 23, 2021
(86) PCT No.: PCT/US2021/047106

§ 371 Date: Feb. 17, 2023

Related U.S. Application Data
(60) Provisional application No. 63/069,632, filed on Aug. 24, 2020.

(51) Int. Cl. C07K 16/28; A61P 37/06

(52) U.S. Cl. CPC C07K 16/283; A61P 37/06

(57) ABSTRACT

Described herein is a method of reducing auto-reactive antibodies in a pregnant individual, an individual trying to become pregnant, an individual considering pregnancy, or an individual serving as a surrogate for the pregnancy, comprising administering an inhibitor of neonatal Fc receptor (FcRn) function to the pregnant individual, the individual trying to become pregnant, the individual considering pregnancy, or the individual serving as a surrogate for the pregnancy.

### CROSS REFERENCE TO RELATED APPLICATIONS

This application is a national stage entry of the PCT application PCT/US2021/047106 filed on Aug. 23, 2021, which claims the benefit of U.S. Provisional Appl. No. 63/069,632 filed on Aug. 24, 2020, each of which is incorporated by reference herein in its entirety.

### SEQUENCE LISTING

This application contains a Sequence Listing which has been submitted electronically in XML format and is hereby incorporated by reference in its entirety. Said XML file, created on Dec. 5, 2023 is named 54669701831_SL.xml and is 6,390 bytes in size.

### BACKGROUND

Autism spectrum disorder (ASD) is a group of heterogeneous neurodevelopmental disorders manifesting in childhood, and defined by communication, behavioral, and social deficiencies. Although strong genetic and environmental links have been demonstrated in numerous reports, a clear etiologic basis for ASD is lacking. Concerning mechanisms that underly ASD etiology, the immune system can play a role in ASD development. Abnormal immune responses, neuroinflammation, and the presence of maternal autoantibodies that target proteins expressed in cells of an embryo or fetus are risk increasing factors for the development of ASD. Notably, such maternal autoantibodies can be present in a pregnant individual, individual trying to become pregnant, or individual considering pregnancy, or an individual serving as a surrogate for the pregnancy, and can result in altered neurological development.

### SUMMARY

Provided herein are methods of reducing auto-reactive antibodies (e.g., an amount present in blood or sera) in an individual that is pregnant, trying to become pregnant, or considering pregnancy. In certain instances, the provided methods for reducing auto-reactive antibodies are based on the discovery that a reduction of auto-reactive antibodies can be achieved by administering an inhibitor of neonatal Fc receptor (FcRn) function, even after a single dose. Such methods generally comprise administering, to an individual that is pregnant, trying to become pregnant, or considering pregnancy, an inhibitor of neonatal Fc receptor (FcRn) function. Practice of the methods disclosed herein facilitates the reduction of maternal auto-reactive antibodies that bind to targets expressed by an embryo or fetus. Such auto-reactive antibodies can affect and/or alter development processes of the embryo or fetus and thus a benefit is conveyed, at least in part, by reducing maternal auto-reactive antibodies, preventing a maternal auto-reactive antibody from contacting an embryo or fetus, or preventing the transport of maternal antibodies across the placenta. Thus, the methods disclosed herein can be used reduce the risk of a child developing ASD or reduce the symptoms of ASD. The methods disclosed herein can also be used reduce the risk or symptoms of ASD associated with the presence of maternal autoantibodies.

Thus, disclosed herein are methods of reducing auto-reactive antibodies (e.g., an amount present in blood or sera) in a pregnant individual or an individual trying to become or an individual considering pregnancy, or an individual serving as a surrogate for the pregnancy, comprising administering, to the pregnant individual or individual trying to become pregnant or individual considering pregnancy, or an individual serving as a surrogate for the pregnancy, an inhibitor of neonatal Fc receptor (FcRn) function.

In some embodiments, the auto-reactive antibodies comprise antibodies that bind a central nervous system (CNS) target. In certain embodiments, an embryo or fetus expresses the CNS target. In some embodiments, the auto-reactive antibodies bind to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In certain embodiments, the auto-reactive antibodies bind to the target LDH-A. In certain embodiments, the auto-reactive antibodies bind to the target LDH-B. In certain embodiments, the auto-reactive antibodies bind to the target GDA. In certain embodiments, the auto-reactive antibodies bind to the target CRMP1. In certain embodiments, the auto-reactive antibodies bind to the target STIP1. In certain embodiments, the auto-reactive antibodies bind to the target DPYSL2. In certain embodiments, the auto-reactive antibodies bind to the target YBX1. In certain embodiments, the auto-reactive antibodies bind to the targetNSE. In certain embodiments, the auto-reactive antibodies bind to the target Caspr2. In certain embodiments, the auto-reactive antibodies bind to the target KCNAB2. In certain embodiments, the auto-reactive antibodies bind to the target KCNAB1. In certain embodiments, the auto-reactive antibodies bind to the target EDIL3. In certain embodiments, the auto-reactive antibodies bind to the target IVD. In certain embodiments, the auto-reactive antibodies bind to the target SUL4A1. In certain embodiments, the auto-reactive antibodies bind to the target TNIP2. In certain embodiments, the auto-reactive antibodies bind to the target RAI16. In certain embodiments, the auto-reactive antibodies bind to the target GDP D5. In some embodiments, the auto-reactive antibodies bind a neural antigen expressed by a fetus.

In some embodiments, the inhibitor of FcRn function prevents and/or blocks auto-reactive antibodies from contacting a fetus or embryo. In certain embodiments, the inhibitor of FcRn function reduces the number of autoreactive antibodies that contact an embryo or fetus. In certain embodiments, the inhibitor of FcRn function inhibits or prevents maternal autoreactive antibodies from crossing the placental barrier. In certain embodiments, the inhibitor of FcRn function inhibits or prevents maternal autoreactive antibodies from crossing the syncytiotrophoblast cell barrier. In certain embodiments, the inhibitor of FcRn function inhibits or prevents maternal autoreactive antibodies from crossing the cytotrophoblast cell barrier. In certain embodiments, the inhibitor of FcRn function inhibits or prevents maternal autoreactive antibodies from crossing the villous stroma.

In some embodiments, the inhibitor prevents an interaction between FcRn and an immunoglobulin G (IgG) molecule. In some embodiments, the inhibitor prevents FcRn-mediated rescue of an IgG molecule. In certain embodiments, the inhibitor prevents an interaction between FcRn and a human serum albumin molecule.

In some embodiments, the inhibitor comprises an antibody or a target-binding fragment thereof, a small molecule, a peptide, or a polypeptide, or a nucleic acid. In some embodiments, the inhibitor is an antibody or target binding fragment thereof. In certain embodiments, the antibody or the target-binding fragment thereof binds FcRn. In certain embodiments, the antibody, or target-binding fragment thereof, is selected from the group consisting of Rozanolixizumab, SYNT001, M281, Argx-113, HL161-11G, HL161-11H, HL161-1A, DX-2504, DX-2507, ABY039, IMVT-1401/RVT1401, and any combination thereof. In certain embodiments, the antibody is Rozanolixizumab. In certain embodiments, the antibody is SYNT001. In certain embodiments, the antibody M281. In certain embodiments, the antibody is Argx-113. In certain embodiments, the antibody is HL161-11G. In certain embodiments, the antibody is DX-2504. In certain embodiments, the antibody is DX-2507. In certain embodiments, the antibody is ABY039,IMVT-1401/RVT1401. In certain embodiments, the antibody or the target-binding fragment thereof comprises a complementarity-determining region of an antibody selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof.

In some embodiments, the inhibitor of FcRn function (e.g., recycling and maintaining of serum antibody levels) is a small molecule. In certain embodiments, the small molecule is selected from TABLE 1. In some embodiments, the inhibitor of FcRn function is a peptide inhibitor. In certain embodiments, the peptide is selected from TABLE 2. In some embodiments, the inhibitor of FcRn function is a protein. In certain embodiments, the protein comprises an Fc region of an IgG molecule. In certain embodiments, the Fc region of an IgG molecule comprises one or more mutations that increase its affinity to FcRn relative to a wild-type Fc molecule.

In some embodiments, the inhibitor of FcRn function (e.g., recycling and maintaining of serum antibody levels) comprises an antibody-binding domain region (e.g., a polypeptide comprising an epitope or an antibody variable domain that binds the Fc region of another antibody) and a variant Fc region, or FcRn-binding fragment thereof, wherein Fc domain of the Fc region or FcRn-binding fragment thereof comprise one or more amino acid substitutions that increase binding to FcRn relative to a wildtype immunoglobulin Fc region. In some embodiments, the inhibitor of FcRn function is a polypeptide comprising a variant Fc region. In certain embodiments, the variant Fc region, or FcRn-binding fragment thereof, comprising the one or more amino acid substitutions that increase binding FcRn (e.g., as described herein) relative to a wild-type IgG1 Fc region. In certain embodiments, the one or more amino acid substitutions comprises Y, T, E, K, F, and Y at EU positions 252, 254, 256, 433, 434, and 436 respectively, and wherein the Fc region binds to FcRn with increased affinity and reduced pH dependence relative to a wild-type IgG1 Fc region. In some embodiments, the inhibitor of FcRn function is an antibody comprising a variant Fc region, wherein the antibody (e.g., an antibody that inhibits FcRn function) comprises a variable domain that recognizes the Fc region of an antibody. In some embodiments, the inhibitor of FcRn function is a fusion polypeptide comprising a polypeptide and a variant Fc region. In certain embodiments, the polypeptide comprises an epitope specific to the auto-reactive antibody.

In some embodiments, the method further comprises comprising assaying a biological sample obtained from the pregnant individual or the individual trying to become pregnant or individual considering pregnancy, or an individual serving as a surrogate for the pregnancy, to detect a maternal antibody that binds to a target selected from the group consisting of a lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In certain embodiments, the biological sample is selected from the group consisting of a maternal blood plasma sample, a maternal blood serum sample, saliva, amniotic fluid, cord blood plasma, cord blood serum sample, fetal blood plasma, fetal blood serum, or a tissue sample. In certain embodiments, a biological sample obtained the pregnant individual or the individual trying to become pregnant or individual considering pregnancy, or an individual serving as a surrogate for the pregnancy, comprises a maternal antibody that binds to a fetal neural antigen. In certain embodiments, the biological sample is a blood plasma sample. In certain embodiments, the biological sample is a blood serum sample. In certain embodiments, the biological sample is a tissue sample.

In some embodiments, the pregnant individual is greater than 30 years old. In some embodiments, the pregnant individual is greater than 35 years old. In some embodiments, the pregnant individual is greater than 40 years old. In some embodiments, the pregnant individual is greater than 45 years old. In some embodiments, the pregnant individual or individual trying to become pregnant has birthed at least one child diagnosed with autism or an autism spectrum disorder.

In some embodiments, a sperm that fertilized an egg of the pregnant individual is from a person greater than 30 years old. In some embodiments, a sperm that fertilized an egg of the pregnant individual is from a person greater than 35 years old. In some embodiments, a sperm that fertilized an egg of the pregnant individual is from a person greater than 40 years old. In some embodiments, a sperm that fertilized an egg of the pregnant individual is from a person greater than 45 years old.

In some embodiments, administering is performed more than once over the course of a pregnancy of the pregnant individual. In some embodiments, administering is performed at least once during each trimester of a pregnancy. In some embodiments, the administering is performed during the first trimester. In some embodiments, the administering is performed during the second trimester. In some embodiments, the administering is performed during the second trimester. In some embodiments, the administering is performed during the third trimester. In some embodiments, the administering is performed during the second trimester. In some embodiments, the administering is performed during the third trimester. In some embodiments, the administering is performed daily. In some embodiments, the administering is performed weekly. In some embodiments, the administering is performed monthly.

In some embodiments, the inhibitor is administered to the pregnant individual before about 30, 60, 90, or 100 days post-conception. In some embodiments, the inhibitor is administered to the pregnant individual after about 100, 150, or 200 days post-conception.

In some embodiments, a gestational age of a fetus of the pregnant individual is less than about 30, 60, 90, or 100 days. In some embodiments, a gestational age of a fetus of the pregnant individual is greater than about 100, 150, or 200 days.

In some embodiments, reducing auto-reactive antibodies (e.g., an amount present in blood or sera) in the pregnant individual or the individual trying to become pregnant prevents or reduces symptoms associated with autism or an autism spectrum disorder. In some embodiments, reducing auto-reactive antibodies in the pregnant individual or the individual trying to become pregnant prevents or reduces symptoms associated with maternal autoantibody related autism spectrum disorder.

Further disclosed herein, are methods of reducing auto-reactive antibodies (e.g., an amount present in blood or sera) in a pregnant individual or an individual trying to become pregnant comprising: (a) obtaining a biological sample obtained from a pregnant individual or an individual considering or trying to become pregnant; (b) determining a presence of a maternal antibody that binds to a fetal neural antigen by at least one assay; (c) administering an inhibitor of neonatal Fc receptor (FcRn) function to the pregnant individual or the individual considering or trying to become pregnant when the presence of the maternal antibody that binds to the fetal neural antigen by the at least one assay is determined.

In some embodiments, the auto-reactive antibodies comprise antibodies that bind a central nervous system (CNS) target. In certain embodiments, an embryo or fetus expresses the CNS target. In some embodiments, the auto-reactive antibodies bind to a target selected from the group consisting of lactate dehydrogenase A (LDH-A), lactate dehydrogenase B (LDH-B), guanine deaminase (GDA), collapsin response mediator protein 1 (CRMP1), stress-induced phosphoprotein 1 (STIP1), a dihydropyrimidinase-like protein 2 (DPYSL2, CRIMP2) protein, a Y Box Binding Protein 1 (YBX1) protein, non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, and any combination thereof. In certain embodiments, the auto-reactive antibodies bind to the target LDH-A. In certain embodiments, the auto-reactive antibodies bind to the target LDH-B. In certain embodiments, the auto-reactive antibodies bind to the target GDA. In certain embodiments, the auto-reactive antibodies bind to the target CRMP1. In certain embodiments, the auto-reactive antibodies bind to the target STIP1. In certain embodiments, the auto-reactive antibodies bind to the target DPYSL2. In certain embodiments, the auto-reactive antibodies bind to the target YBX1. In certain embodiments, the auto-reactive antibodies bind to the targetNSE. In certain embodiments, the auto-reactive antibodies bind to the target Caspr2. In certain embodiments, the auto-reactive antibodies bind to the target KCNAB2. In certain embodiments, the auto-reactive antibodies bind to the target KCNAB1. In certain embodiments, the auto-reactive antibodies bind to the target EDIL3. In certain embodiments, the auto-reactive antibodies bind to the target IVD. In certain embodiments, the auto-reactive antibodies bind to the target SUL4A1. In certain embodiments, the auto-reactive antibodies bind to the target TNIP2. In certain embodiments, the auto-reactive antibodies bind to the target RAI16. In certain embodiments, the auto-reactive antibodies bind to the target GDP D5. In some embodiments, the auto-reactive antibodies bind a neural antigen expressed by a fetus.

In some embodiments, the assay is an immune assay. In some embodiments, the biological sample is selected from the group consisting of a maternal blood plasma sample, a maternal blood serum sample, saliva, amniotic fluid, cord blood plasma, cord blood serum sample, fetal blood plasma, fetal blood serum, or a tissue sample. In certain embodiments, a biological sample obtained the pregnant individual or the individual trying to become pregnant or individual considering pregnancy, or an individual serving as a surrogate for the pregnancy, comprises a maternal antibody that binds to a fetal neural antigen. In certain embodiments, the biological sample is a blood plasma sample. In certain embodiments, the biological sample is a blood serum sample. In certain embodiments, the biological sample is a tissue sample.

In some embodiments, the assay detects the presence and/or levels of a maternal antibody that binds to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In certain embodiments, the auto-reactive antibodies bind to the target LDH-A. In certain embodiments, the maternal antibody binds to the target LDH-B. In certain embodiments, the maternal antibody binds to the target GDA. In certain embodiments, the maternal antibody binds to the target CRMP1. In certain embodiments, the maternal antibody binds to the target STIP1. In certain embodiments, the maternal antibody binds to the target DPYSL2. In certain embodiments, the maternal antibody binds to the target YBX1. In certain embodiments, the maternal antibody binds to the target NSE. In certain embodiments, the maternal antibody binds to the target Caspr2. In certain embodiments, the maternal antibody binds to the target KCNAB2. In certain embodiments, the maternal antibody binds to the target KCNAB1. In certain embodiments, the maternal antibody binds to the target EDIL3. In certain embodiments, the maternal antibody binds to the target IVD. In certain embodiments, the maternal antibody binds to the target SUL4A1. In certain embodiments, the maternal antibody binds to the target TNIP2. In certain embodiments, the maternal antibody binds to the target RAI16. In certain embodiments, the maternal antibody binds to the target GDP D5. In some embodiments, the maternal antibody binds a neural antigen expressed by a fetus.

In some embodiments, the inhibitor of FcRn function prevents and/or blocks auto-reactive antibodies from contacting a fetus or embryo. In certain embodiments, the inhibitor of FcRn function reduces the number of autoreactive antibodies that contact an embryo or fetus. In certain embodiments, the inhibitor of FcRn function inhibits or prevents maternal autoreactive antibodies from crossing the placental barrier. In certain embodiments, the inhibitor of FcRn function inhibits or prevents maternal autoreactive antibodies from crossing the syncytiotrophoblast cell barrier. In certain embodiments, the inhibitor of FcRn function inhibits or prevents maternal autoreactive antibodies from crossing the cytotrophoblast cell barrier. In certain embodiments, the inhibitor of FcRn function inhibits or prevents maternal autoreactive antibodies from crossing the villous stroma.

In some embodiments, the inhibitor prevents an interaction between FcRn and an immunoglobulin G (IgG) molecule. In some embodiments, the inhibitor prevents FcRn-mediated rescue of an IgG molecule. In certain embodiments, the inhibitor prevents an interaction between FcRn and a human serum albumin molecule.

In some embodiments, the inhibitor comprises an antibody or a target-binding fragment thereof, a small molecule, a peptide, or a polypeptide, or a nucleic acid. In some embodiments, the inhibitor is an antibody or target binding fragment thereof. In certain embodiments, the antibody or the target-binding fragment thereof binds FcRn. In certain embodiments, the antibody, or target-binding fragment thereof, is selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof. In certain embodiments, the antibody is Rozanolixizumab. In certain embodiments, the antibody is SYNT001. In certain embodiments, the antibody M281. In certain embodiments, the antibody is Argx-113. In certain embodiments, the antibody is HL161-11G. In certain embodiments, the antibody is DX-2504. In certain embodiments, the antibody is DX-2507. In certain embodiments, the antibody is ABY039,IMVT-1401/RVT1401. In certain embodiments, the antibody or the target-binding fragment thereof comprises a complementarity-determining region of an antibody selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof.

In some embodiments, the inhibitor of FcRn function is a small molecule. In certain embodiments, the small molecule is selected from TABLE 1. In some embodiments, the inhibitor of FcRn function is a peptide inhibitor. In certain embodiments, the peptide is selected from TABLE 2. In some embodiments, the inhibitor of FcRn function is a protein. In certain embodiments, the protein comprises an Fc region of an IgG molecule. In certain embodiments, the Fc region of an IgG molecule comprises one or more mutations that increase its affinity to FcRn relative to a wild-type Fc molecule.

In some embodiments, the pregnant individual is greater than 30 years old. In some embodiments, the pregnant individual is greater than 35 years old. In some embodiments, the pregnant individual is greater than 40 years old. In some embodiments, the pregnant individual is greater than 45 years old. In some embodiments, the pregnant individual or individual trying to become pregnant has birthed at least one child diagnosed with autism or an autism spectrum disorder.

In some embodiments, a sperm that fertilized an egg of the pregnant individual is from a person greater than 30 years old. In some embodiments, a sperm that fertilized an egg of the pregnant individual is from a person greater than 35 years old. In some embodiments, a sperm that fertilized an egg of the pregnant individual is from a person greater than 40 years old. In some embodiments, a sperm that fertilized an egg of the pregnant individual is from a person greater than 45 years old.

In some embodiments, administering is performed more than once over the course of a pregnancy of the pregnant individual. In some embodiments, administering is performed at least once during each trimester of a pregnancy. In some embodiments, the administering is performed during the first trimester. In some embodiments, the administering is performed during the second trimester. In some embodiments, the administering is performed during the second trimester. In some embodiments, the administering is performed during the third trimester. In some embodiments, the administering is performed during the second trimester. In some embodiments, the administering is performed during the third trimester. In some embodiments, the administering is performed daily. In some embodiments, the administering is performed weekly. In some embodiments, the administering is performed monthly.

In some embodiments, the inhibitor is administered to the pregnant individual before about 30, 60, 90, or 100 days post-conception. In some embodiments, the inhibitor is administered to the pregnant individual after about 100, 150, or 200 days post-conception.

In some embodiments, a gestational age of a fetus of the pregnant individual is less than about 30, 60, 90, or 100 days. In some embodiments, a gestational age of a fetus of the pregnant individual is greater than about 100, 150, or 200 days.

In some embodiments, reducing auto-reactive antibodies in the pregnant individual or the individual trying to become pregnant prevents or reduces symptoms associated with autism or an autism spectrum disorder. In some embodiments, reducing auto-reactive antibodies in the pregnant individual or the individual trying to become pregnant prevents or reduces symptoms associated with maternal autoantibody related autism spectrum disorder.

Further disclosed herein, is the use of an inhibitor of neonatal Fc receptor (FcRn) function in methods for reducing auto-reactive antibodies in a pregnant individual or an individual considering or trying to become pregnant. Also disclosed herein, is the use of an inhibitor of neonatal Fc receptor (FcRn) function in the manufacture of a medicament for the treatment of auto-reactive antibodies in a pregnant individual or an individual trying to become pregnant.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary schematic for use of an FcRn inhibitor to reduce auto-reactive antibodies in a pregnant individual.

FIG. 2 shows an exemplary schematic for use of an FcRn inhibitor to reduce auto-reactive antibodies in a pregnant individual.

FIG. 3 shows an exemplary schematic for use of an FcRn inhibitor to reduce auto-reactive antibodies in an individual trying to become pregnant or considering pregnancy.

FIG. 4 shows an exemplary schematic for use of an FcRn inhibitor to reduce auto-reactive antibodies in an individual trying to become pregnant or considering pregnancy.

FIG. 5 shows an exemplary schematic for use of an FcRn inhibitor to reduce auto-reactive antibodies in an individual serving as a pregnancy surrogate.

FIGS. 6A, 6B, 6C, 6D, and 6E show in vivo data demonstrating a reduction in autoantibodies after administering an inhibitor of FcRn function.

### DETAILED DESCRIPTION

Provided herein are methods useful in inhibiting the function of the neonatal Fc receptor (FcRn). Such methods are used to reduce maternal auto-reactive antibodies (autoantibodies) within a pregnant individual, an individual trying to become pregnant, an individual considering pregnancy, or an individual serving as a surrogate for the pregnancy. Generally, the reduction of auto-reactive antibodies can be used in preventing and/or decreasing the likelihood of a child having Autism Spectrum Disorder (ASD).

Disclosed herein are methods reducing maternal autoantibodies that target neurodevelopmental proteins. Through the practice of the disclosure herein, inhibition of FcRn that prevents maternal antibodies from contacting an embryo or fetus can be used in preventing and/or decreasing the likelihood of maternal autoantibody related (MAR) ASD in a child. In certain instances, inhibition of FcRn function can reduce the number of maternal autoantibodies. In such instances, the reduction of maternal antibodies through inhibition of FcRn is useful for preventing and/or decreasing the likelihood of MAR ASD in a child. Thus, the use and/or practice of the methods for reducing maternal autoantibodies consistent with the disclosure herein facilitates the broad application of FcRn inhibitors to prevent, reduce, and/or treat MAR ASD in a child.

Maternal autoantibodies can be reduced by administering an inhibitor of FcRn function to a pregnant individual, an individual trying to become pregnant, an individual considering pregnancy, or an individual serving as a surrogate for the pregnancy. This is done through the administration of any number of FcRn inhibitors, including known FcRn inhibitors. Diagnostic screening methods, tests, and kits can be used in methods for reducing maternal autoantibodies. For example, the presence or absence of maternal autoantibodies can be determined in a pregnant individual, an individual trying to become pregnant, an individual considering pregnancy, or an individual serving as a surrogate for the pregnancy, wherein the results of diagnostic methods can inform various parameters for the administration of FcRn inhibitors before or during pregnancy.

The methods described herein can provide several advantages. For example, the methods identify a druggable target, FcRn, for the prevention of ASD, a disease associated with complex causation. Assessing the FcRn target allows for the application and/or development of inhibitors that reduce FcRn function in auto-reactive maternal antibodies and/or prevent auto-reactive maternal antibodies from contacting an embryo or fetus. As a result, the disclosure herein substantially facilitates the application of methods to reduce maternal autoantibodies, prevent auto-reactive maternal antibodies from contacting an embryo or fetus, and/or prevent ASD. As another example, the use of diagnostics in the methods of inhibiting FcRn function can provide a readout for informing and/or directing treatment or prevention of MAR ASD.

### Autism

The methods provided herein can reduce the risk of ASD or MAR ASD by inhibition of FcRn function in a pregnant individual, individual trying to become pregnant, an individual considering pregnancy, or an individual serving as a surrogate for the pregnancy. The terms “autism spectrum disorder” and “autistic spectrum disorder” and “autism” and “ASD” interchangeably refer to a spectrum of neurodevelopmental disorders characterized by impaired social interaction and communication accompanied by repetitive and stereotyped behavior. The term “MAR ASD” refers to ASD associated with the presence of maternal autoantibodies, correlated with the presence of maternal autoantibodies, and/or caused by the presence of maternal autoantibodies. Autism includes a spectrum of impaired social interaction and communication, however, the disorder can be roughly categorized into “high functioning autism” or “low functioning autism,” depending on the extent of social interaction and communication impairment. Individuals diagnosed with “high functioning autism” have minimal but identifiable social interaction and communication impairments (i.e., Asperger's syndrome). Additional information on autism spectrum disorders can be found in, for example, Autism Spectrum Disorders: A Research Review for Practitioners, Ozonoff, et al., eds., 2003, American Psychiatric Pub; Gupta, Autistic Spectrum Disorders in Children, 2004, Marcel Dekker Inc; Hollander, Autism Spectrum Disorders, 2003, Marcel Dekker Inc; Handbook of Autism and Developmental Disorders, Volkmar, ed., 2005, John Wiley; Sicile-Kira and Grandin, Autism Spectrum Disorders: The Complete Guide to Understanding Autism, Asperger's Syndrome, Pervasive Developmental Disorder, and Other ASDs, 2004, Perigee Trade; and Duncan, et al., Autism Spectrum Disorders [Two Volumes]: A Handbook for Parents and Professionals, 2007, Praeger.

The term “increased risk of developing an ASD” refers to an increased likelihood or probability that a fetus or child exposed to antibodies that bind to one or more biomarkers described herein (e.g., selected from lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), or any combination thereof) or to levels of antibodies against the one or more of the biomarkers above a predetermined threshold level will develop symptoms of an ASD in comparison to the risk, likelihood or probability of a fetus or child that has not been exposed to antibodies against the one or more biomarkers or to levels of antibodies against the one or more biomarkers that are below a predetermined threshold level.

The term “reduced risk of developing an ASD” refers to the decreased likelihood or probability that a fetus or child exposed to antibodies against one or more biomarkers described herein (e.g., selected lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), or any combination thereof) or to levels of antibodies against the one or more of the biomarkers above a predetermined threshold level, and whose mother has received therapeutic intervention, e.g., to block, inactivate or remove antibodies that bind to the biomarkers, will develop symptoms of an ASD in comparison to the likelihood or probability that a fetus or child exposed to antibodies against the biomarkers or to levels of antibodies against the one or more biomarkers above a predetermined threshold level and whose mother has not received therapeutic intervention will develop symptoms of an ASD.

### FcRn

The neonatal Fc-receptor (FcRn) is important for in vivo metabolic regulation of antibodies of the immunoglobulin class G (IgG). The FcRn functions to rescue or prevent IgG antibodies from the cellular lysosomal degradation pathway, resulting in reduced clearance and increased half-life. FcRn as referred to herein refers to the non-covalent complex between the human IgG receptor alpha chain, also known as the neonatal Fc receptor (FcRn), the amino acid sequence of which is disclosed in UniProt under number P55899 and β2 microglobulin (β2M), the amino acid sequence of which is in UniProt under number P61769. FcRn is a heterodimeric protein consisting of two polypeptides: a 50 kDa class I major histocompatibility complex-like protein (α-FcRn) and a 15 kDa β2-microglobulin (β2m). (see e.g. Huber et al. (1993) J. Mol. Biol. 230, 1077-1083).

FcRn function generally refers to the cellular internalization and recycling of IgG via FcRn, a process that can lead to the maintenance of IgG levels in vivo. FcRn binds with high affinity to the CH2-CH3 portion of the Fc-region of an antibody of the class IgG. The interaction between an antibody of the class IgG and the FcRn can be pH dependent and can occur in a 1:2 stoichiometry wherein one IgG antibody molecule can interact with two FcRn molecules via its two heavy chain Fc-region polypeptides. In some embodiments, inhibiting function of FcRn comprises inhibiting rescue or recycling of an auto-reactive antibody where, in certain instances, rescue or recycling of an auto-reactive antibodies promotes the maintenance of IgG levels in an individual. In some embodiments, inhibiting function of FcRn comprises inhibiting rescue or recycling of an auto-reactive antibody where, in certain instances, rescue or recycling of an auto-reactive antibody increases the half-life of an auto-reactive antibody. In certain embodiments, inhibiting FcRn function comprises inhibiting an interaction between FcRn and an auto-reactive antibody (e.g, using antibody, small molecules, peptides, or proteins). In certain embodiments, inhibiting FcRn function comprises targeting an auto-reactive antibody for degradation by enhancing an interaction (e.g., indirect interaction) with FcRn, thereby shuttling the auto-reactive antibody into a degradation pathway.

Among many other functions, FcRn can be essential for the transfer of humoral immunity to the fetus (see e.g. Roopenian et al., Nat Rev Immunol. 2007 September; 7(9):715-25). FcRn function, at least in part, further refers to the transfer of maternal IgG antibodies to the fetus via the placenta from early- to mid-gestation onwards by an FcRn-mediated active transport mechanism, wherein maternal IgG binds to the FcRn expressed in endosomal vesicles in the syncytiotrophoblast of the placenta and is then released into the fetal circulation.

Methods as disclosed herein for reducing auto-reactive antibodies (e.g., an amount present in blood or sera) capable of contacting an embryo and/or fetus of a pregnant individual, an individual trying to become pregnant, an individual considering pregnancy, or an individual serving as a surrogate for the pregnancy, are characterized by the inhibition of FcRn function within the pregnant individual, individual trying to become pregnant, or considering pregnancy. Such methods, as disclosed herein, can also be used for reducing auto-reactive antibodies (e.g., an amount present in blood or sera) in a pregnant individual, an individual trying to become pregnant, an individual considering pregnancy, or an individual serving as a surrogate for the pregnancy. In some embodiments, inhibiting FcRn function comprises inhibiting transfer of maternal antibodies across a placental barrier. In certain embodiments, inhibiting FcRn function comprises inhibiting transfer of maternal antibodies to a region wherein the antibodies can contact an embryo or fetus. In certain embodiments, inhibiting FcRn function comprises inhibiting FcRn-mediated IgG transfer across or through endosomal vesicles in the syncytiotrophoblast of the placenta. In some embodiments, inhibiting FcRn function comprises inhibiting or blocking the interaction between FcRn and an antibody molecule. In some embodiments, inhibiting FcRn function comprises inhibiting, blocking, and/or reducing FcRn-mediated recycling or rescue of an antibody molecule. In some embodiments, inhibiting FcRn function comprises inhibiting accessory molecules involved in FcRn function. In certain embodiments, inhibiting FcRn function comprises inhibiting or blocking the interaction between FcRn and a human serum albumin molecule.

Thus, as it can be understood, an IgGs FcRn binding properties/characteristics are indicative of its in vivo pharmacokinetic properties in the blood circulation. n the interaction between the FcRn and the Fc-region of an antibody of the IgG class different amino acid residues of the heavy chain CH2- and CH3-domain are participating. The amino acid residues interacting with the FcRn are located approximately between EU position 243 and EU position 261, approximately between EU position 275 and EU position 293, approximately between EU position 302 and EU position 319, approximately between EU position 336 and EU position 348, approximately between EU position 367 and EU position 393, at EU position 408, and approximately between EU position 424 and EU position 440. More specifically the following amino acid residues according to the EU numbering of Kabat are involved in the interaction between the Fc-region and the FcRn: F243, P244, P245 P, K246, P247, K248, D249, T250, L251, M252, I253, S254, R255, T256, P257, E258, V259, T260, C261, F275, N276, W277, Y278, V279, D280, V282, E283, V284, H285, N286, A287, K288, T289, K290, P291, R292, E293, V302, V303, 5304, V305, L306, T307, V308, L309, H310, Q311, D312, W313, L314, N315, G316, K317, E318, Y319, I336, S337, K338, A339, K340, G341, Q342, P343, R344, E345, P346, Q347, V348, C367, V369, F372, Y373, P374, S375, D376, I377, A378, V379, E380, W381, E382, S383, N384, G385, Q386, P387, E388, N389, Y391, T393, S408, S424, C425, S426, V427, M428, H429, E430, A431, L432, H433, N434, H435, Y436, T437, Q438, K439, and S440. Site-directed mutagenesis studies have proven that the critical binding sites in the Fc-region of IgGs for FcRn are Histidine 310, Histidine 435, and Isoleucine 253 and to a lesser extent Histidine 433 and Tyrosine 436 (see e.g. Kim, J. K., et al., Eur. J. Immunol. 29 (1999) 2819-2825; Raghavan, M., et al., Biochem. 34 (1995) 14649-14657; Medesan, C., et al., J Immunol. 158 (1997) 2211-2217). Methods to increase IgG binding to FcRn have been performed by mutating IgG at various amino acid residues: Threonine 250, Methionine 252, Serine 254, Threonine 256, Threonine 307, Glutamic acid 380, Methionine 428, Histidine 433, and Asparagine 434 (see Kuo, T. T., et al., J. Clin. Immunol. 30 (2010) 777-789).

### Maternal Auto-Reactive Antibodies and Detection Thereof

Auto-reactive antibodies in a pregnant individual, an individual trying to become pregnant, an individual considering pregnancy, or an individual serving as a surrogate for the pregnancy, can cause or affect development of an embryo or fetus. As an example, auto-reactive antibodies of a pregnant individual, an individual trying to become pregnant, an individual considering pregnancy, or an individual serving as a surrogate for the pregnancy, can affect or alter neural development of an embryo or fetus. In another example, auto-reactive antibodies of a pregnant individual, an individual trying to become pregnant, an individual considering pregnancy, or an individual serving as a surrogate for the pregnancy, can lead to a child developing ASD.

The term “auto-reactive antibody” is used herein interchangeably with the term “autoantibody” and describes an antibody that can target or bind one or more proteins or antigens expressed by a developing embryo and/or fetus. Accordingly, in some embodiments, the auto-reactive antibodies bind a target expressed by or associated with an embryo or fetus. In various embodiments, the auto-reactive antibodies bind a neural antigen expressed by a fetus. In some embodiments, the auto-reactive antibodies comprise antibodies that bind a central nervous system (CNS) target. In some embodiments, an embryo or fetus expresses the CNS target. In some embodiments, the auto-reactive antibodies bind to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), or any combination thereof.

Methods as disclosed herein for reducing auto-reactive antibodies (e.g., an amount present in blood or sera) in a pregnant individual, an individual trying to become pregnant, an individual considering pregnancy, or an individual serving as a surrogate for the pregnancy, are characterized by the inhibition of FcRn function within the pregnant individual, individual trying to become pregnant, or considering pregnancy can comprise a diagnostic method, step, or test to determine the presence and/or quantification of auto-reactive antibodies. In exemplary embodiments, the method reducing auto-reactive antibodies in a pregnant individual or an individual trying to become pregnant comprises: (a) obtaining or providing a biological sample obtained from a pregnant individual or an individual considering or trying to become pregnant; (b) determining a presence of a maternal antibody that binds to a fetal neural antigen by at least one assay; and (c) administering an inhibitor of neonatal Fc receptor (FcRn) function to the pregnant individual or the individual considering or trying to become pregnant when the presence of the maternal antibody that binds to the fetal neural antigen by the at least one assay is determined. In various embodiments, determining a presence of a maternal antibody that binds to a fetal neural antigen by at least one assay is performed prior to an individual becoming pregnant. In some embodiments, determining a presence of a maternal antibody that binds to a fetal neural antigen by at least one assay is performed prior to an individual trying to become pregnant. In some embodiments, determining a presence of a maternal antibody that binds to a fetal neural antigen by at least one assay is performed while to an individual is considering pregnancy. In various embodiments, determining a presence of a maternal antibody that binds to a fetal neural antigen by at least one assay is performed after an individual becomes pregnant. In some embodiments, determining a presence of a maternal antibody that binds to a fetal neural antigen by at least one assay is performed after an individual begins trying to become pregnant.

Additionally, methods disclosed herein for reducing auto-reactive antibodies (e.g., an amount present in blood or sera) in a pregnant individual, an individual trying to become pregnant, an individual considering pregnancy, or an individual serving as a surrogate for the pregnancy, comprise a diagnostic method, step, or test to determine the presence and/or quantification of auto-reactive antibodies. In exemplary embodiments, the method reducing auto-reactive antibodies in a pregnant individual or an individual trying to become pregnant comprises: (a) obtaining a biological sample obtained from a pregnant individual or an individual considering or trying to become pregnant; (b) determining a presence of a maternal antibody that binds to a fetal neural antigen by at least one assay; and (c) administering an inhibitor of neonatal Fc receptor (FcRn) function to the pregnant individual or the individual considering or trying to become pregnant when the presence of the maternal antibody that binds to the fetal neural antigen by the at least one assay is determined. In various embodiments, determining a presence of a maternal antibody that binds to a fetal neural antigen by at least one assay is performed prior to an individual becoming pregnant. In some embodiments, determining a presence of a maternal antibody that binds to a fetal neural antigen by at least one assay is performed prior to an individual trying to become pregnant. In some embodiments, determining a presence of a maternal antibody that binds to a fetal neural antigen by at least one assay is performed while to an individual is considering pregnancy. In various embodiments, determining a presence of a maternal antibody that binds to a fetal neural antigen by at least one assay is performed after an individual becomes pregnant. In some embodiments, determining a presence of a maternal antibody that binds to a fetal neural antigen by at least one assay is performed after an individual begins trying to become pregnant.

Methods for detecting auto-reactive antibodies in a pregnant individual or an individual trying or considering pregnancy can comprise identifying in a biological sample from the pregnant individual, or the individual trying or considering pregnancy, the presence of maternal antibodies that bind to one or more of the biomarkers described herein (e.g., selected from lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), or any combination thereof), wherein the presence of maternal antibodies that specifically bind to the one or more biomarkers indicates an increased likelihood of the fetus or child for developing an ASD. In another embodiment, the method comprises determining the likelihood that a fetus or child will develop an autism spectrum disorder (ASD) by the presence of maternal antibodies that bind to one or more of the biomarkers described herein

With respect to the biological sample taken from the mother, any fluid sample containing antibodies can be used. For example, the biological sample may be blood, serum, plasma, amniotic fluid, urine, milk, or saliva. Of course, one or more different bodily fluids can be evaluated for antibodies that specifically bind to the one or more biomarkers.

The biological sample is evaluated for the presence of antibodies that specifically bind to bind to 1, 2, 3, 4, 5, 6, 7, 8, 9, 10, 11, 12, 13, or more, of the biomarkers (e.g., selected from lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), or any combination thereof)

The terms “lactate dehydrogenase” or “LDH” interchangeably refer to an enzyme that catalyzes the interconversion of pyruvate and lactate with concomitant interconversion of NADH and NAD+. Lactate dehydrogenases exist in four distinct enzyme classes. Two of them are cytochrome c-dependent enzymes with each acting on either D-lactate (EC 1.1.2.4) or L-lactate (EC 1.1.2.3). The other two are NAD(P)-dependent enzymes with each acting on either D-lactate (EC 1.1.1.28) or L-lactate (EC 1.1.1.27). The LDH enzyme is composed of 4 subunits, wherein the subunits are either “M” or “H”. The LDHA gene encodes the M subunit, known interchangeably LDH-M or LDH-A. The LDHB gene encodes the H subunit, known interchangeably as LDH-H or LDH-B. There are five LDH isozymes, each containing four subunits. The major LDH isozyme of skeletal muscle and liver, LDH-5 (M4), has four muscle (M) subunits; while LDH-1 (H4) is the main isozyme for heart muscle in most species, containing 4 heart (H) subunits. The other variants contain both types of subunits, e.g., LDH-2 (H3M1)—in the reticuloendothelial system, LDH-3 (H2M2)—in the lungs, and LDH-4 (H1M3)—in the kidneys. LDH-2 is the predominant form in the serum. LDHA is also known as LDH1, LDH muscle subunit, LDH-M, EC 1.1.1.27, Renal carcinoma antigen NY-REN-59, Cell proliferation-inducing gene 19 protein, PIG19 and L-lactate dehydrogenase A chain; LDHB is also known as LDH2 or LDH-H or TRG-5; LDHC is testis specific.

Structurally, an LDH-A amino acid sequence has at least about 90%, 91%, 92%, 93%, 94%, 95%, 96%, 97%, 98%, 99% or 100% sequence identity with the amino acid sequence, e.g., of GenBank accession no. AAP36496.1; BAD96798.1; NM-005566.3→NP-005557.1 (isoform 1); NM-001135239.1→NP-001128711.1 (isoform 2); NM-001165414.1→NP-001158886.1 (isoform 3); NM-001165415.1→NP-001158887.1 (isoform 4); or NM-001165416.1→NP-001158888.1 (isoform 5) over a sequence length of at least 50, 100, 150, 200, 250, 300, 350 amino acids or over the full length of the polypeptide. Structurally, an LDH-A nucleic acid sequence has at least about 90%, 91%, 92%, 93%, 94%, 95%, 96%, 97%, 98%, 99% or 100% sequence identity with the nucleic acid sequence, e.g., of GenBank accession no. BC067223; CR604911; BC051361; X02152.1; NM-005566.3→NP-005557.1 (isoform 1); NM-001135239.1→NP-001128711.1 (isoform 2); NM-001165414.1→NP-001158886.1 (isoform 3); NM-001165415.1→NP-001158887.1 (isoform 4); or NM-001165416.1→NP-001158888.1 (isoform 5) over a sequence length of at least 300, 500, 750, 1000 nucleic acids or over the full length of the polynucleotide. The sequence alignments can be performed using any alignment algorithm known in the art, e.g., BLAST, ALIGN, set to default settings.

Structurally, an LDH-B amino acid sequence has at least about 90%, 91%, 92%, 93%, 94%, 95%, 96%, 97%, 98%, 99% or 100% sequence identity with the amino acid sequence, e.g., of GenBank accession no. NM-002300.6→NP-002291.1 (variant 1); or NM-001174097.1→NP-001167568.1 (variant 2) over a sequence length of at least 50, 100, 150, 200, 250, 300, 350 amino acids or over the full length of the polypeptide. Structurally, an LDH-B nucleic acid sequence has at least about 90%, 91%, 92%, 93%, 94%, 95%, 96%, 97%, 98%, 99% or 100% sequence identity with the nucleic acid sequence, e.g., of GenBank accession no. BC002361.1; Y00711.1; NM-002300.6→NP-002291.1 (variant 1); or NM-001174097.1→NP-001167568.1 (variant 2) over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “collapsin response mediator protein 1” or “CRMP1” (also known as DRP1; DRP-1; CRMP-1; DPYSL1; ULIP-3) refer to a cytosolic phosphoprotein known to function in neuronal differentiation and axonal guidance. CRMP1 is a member of a family of cytosolic phosphoproteins expressed exclusively in the nervous system. The encoded protein is thought to be a part of the semaphorin signal transduction pathway implicated in semaphorin-induced growth cone collapse during neural development. Alternative splicing results in multiple transcript variants. Structurally, a CRMP1 amino acid sequence has at least about 90%, 91%, 92%, 93%, 94%, 95%, 96%, 97%, 98%, 99% or 100% sequence identity with the amino acid sequence, e.g., of GenBank accession no. NM-001014809.1→NP-001014809.1 (isoform 1) or NM-001313.3→NP-001304.1 (isoform 2), over a sequence length of at least 50, 100, 150, 200, 250, 300, 350 amino acids or over the full length of the polypeptide. Structurally, a CRMP1 nucleic acid sequence has at least about 90%, 91%, 92%, 93%, 94%, 95%, 96%, 97%, 98%, 99% or 100% sequence identity with the nucleic acid sequence, e.g., of GenBank accession no. NM-001014809.1→NP-001014809.1 (isoform 1) or NM-001313.3→NP-001304.1 (isoform 2), over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “Stress Induced Phosphoprotein 1” or “STIP1” (also known as Hsp70/Hsp90-organizing Protein (HOPI), STI1, STILL IEF-SSP-3521 and P60) refers to an adaptor protein that assists in folding of HSP70 and HSP90. STIP1 also stimulates the ATPase activity of HSP70, while inhibiting the ATPase activity of HSP90, suggesting a regulatory role. Furthermore, STIP1 binds to the cellular prion protein PrPc and regulates short-term and long-term memory consolidation. Structurally, a STIP1 amino acid sequence has at least about 90%, 91%, 92%, 93%, 94%, 95%, 96%, 97%, 98%, 99% or 100% sequence identity with the amino acid sequence, e.g., of GenBank accession no. NM-006819.2→NP-006810.1, over a sequence length of at least 50, 100, 150, 200, 250, 300, 350 amino acids or over the full length of the polypeptide. Structurally, a STIP1 nucleic acid sequence has at least about 90%, 91%, 92%, 93%, 94%, 95%, 96%, 97%, 98%, 99% or 100% sequence identity with the nucleic acid sequence, e.g., of GenBank accession no. NM-006819.2→NP-006810.1, over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “guanine deaminase” and “GDA” (also known as Cypin, Guanase, KIAA1258, MGC9982 and Nedasin) refers to an enzyme that catalyzes the hydrolytic deamination of guanine, yielding xanthine and ammonia. GDA has also been shown to regulate PSD-95 postsynaptic targeting. Structurally, a GDA amino acid sequence has at least about 90%, 91%, 92%, 93%, 94%, 95%, 96%, 97%, 98%, 99% or 100% sequence identity with the amino acid sequence, e.g., of GenBank accession no. NM-004293.3→NP-004284.1, over a sequence length of at least 50, 100, 150, 200, 250, 300, 350 amino acids or over the full length of the polypeptide. Structurally, a GDA nucleic acid sequence has at least about 90%, 91%, 92%, 93%, 94%, 95%, 96%, 97%, 98%, 99% or 100% sequence identity with the nucleic acid sequence, e.g., of GenBank accession no. NM-004293.3→NP-004284.1, over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “Dihydropyrimidinase-like Protein 2” or “DPYSL2” (also known as CRMP-2 or CRMP2) refer to a protein involved in axonal guidance by mediating the repulsive effect of Sema3A in axons during axonal specification. Structurally, a DPYSL2 amino acid sequence has at least about 90%, 91%, 92%, 93%, 94%, 95%, 96%, 97%, 98%, 99% or 100% sequence identity with the amino acid sequence, e.g., of GenBank accession no. NM-001386.4→NP-001377.1 or BAD92432, over a sequence length of at least 50, 100, 150, 200, 250, 300, 350 amino acids or over the full length of the polypeptide. Structurally, a DPYSL2 nucleic acid sequence has at least about 90%, 91%, 92%, 93%, 94%, 95%, 96%, 97%, 98%, 99% or 100% sequence identity with the nucleic acid sequence, e.g., of GenBank accession no. NM-001386.4→NP-001377.1, over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “alpha subunit of the barbed-end actin binding protein Cap Z” or “capping protein (actin filament) muscle Z-line, alpha 2” or “CAPZA2” (also known as CAPPA2, CAPZ) refer to a member of the F-actin capping protein alpha subunit family. CAPZA2 is the alpha subunit of the barbed-end actin binding protein Cap Z. By capping the barbed end of actin filaments, Cap Z regulates the growth of the actin filaments at the barbed end. Structurally, a CAPZA2 amino acid sequence has at least about 90%, 91%, 92%, 93%, 94%, 95%, 96%, 97%, 98%, 99% or 100% sequence identity with the amino acid sequence, e.g., of GenBank accession no. NM-006136.2→NP-006127.1, over a sequence length of at least 50, 100, 150, 200, 250, 300, 350 amino acids or over the full length of the polypeptide. Structurally, a CAPZA2 nucleic acid sequence has at least about 90%, 91%, 92%, 93%, 94%, 95%, 96%, 97%, 98%, 99% or 100% sequence identity with the nucleic acid sequence, e.g., of GenBank accession no. NM-006136.2→NP-006127.1, over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “Y Box Binding Protein 1” or “YBX1” (also known as BP-8, CSDA2, CSDB, DBPB, MDR-NF1, MGC104858, MGC110976, MGC117250, NSEP-1, NSEP1, YB1 and YB1) refers to a protein that mediates pre-mRNA alternative splicing regulation. YBX1 binds to splice sites in pre-mRNA and regulates splice site selection; binds and stabilizes cytoplasmic mRNA; contributes to the regulation of translation by modulating the interaction between the mRNA and eukaryotic initiation factors; binds to promoters that contain a Y-box (5′-CTGATTGGCCAA-3′; SEQ ID NO. 4), e.g., found in HLA class II genes; and promotes the separation of DNA strands that contain mismatches or are modified by cisplatin. Structurally, a YBX1 amino acid sequence has at least about 90%, 91%, 92%, 93%, 94%, 95%, 96%, 97%, 98%, 99% or 100% sequence identity with the amino acid sequence, e.g., of GenBank accession no. NM-004559.3→NP-004550.2, over a sequence length of at least 50, 100, 150, 200, 250, 300, 350 amino acids or over the full length of the polypeptide. Structurally, a YBX1 nucleic acid sequence has at least about 90%, 91%, 92%, 93%, 94%, 95%, 96%, 97%, 98%, 99% or 100% sequence identity with the nucleic acid sequence, e.g., of GenBank accession no. NM-004559.3→NP-004550.2, over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “Eukaryotic Translation and Elongation Factor 1A1” and “EEF1A1” refer to an isoform of the alpha subunit of the elongation factor-1 complex that transports aminoacyl tRNA's to the ribosome. The 1A1 isoform is expressed in brain, placenta, lung, liver and pancreas and is an autoantigen in 66% of embodiments of Felty syndrome. Felty syndrome is characterized by a combination of rheumatoid arthritis, splenomegaly and neutropenia. Structurally, an EEF1A1 amino acid sequence has at least about 90%, 91%, 92%, 93%, 94%, 95%, 96%, 97%, 98%, 99% or 100% sequence identity with the amino acid sequence, e.g., of GenBank accession no. NM-001402.5→NP-001393.1, over a sequence length of at least 50, 100, 150, 200, 250, 300, 350 amino acids or over the full length of the polypeptide. Structurally, an EEF1A1 nucleic acid sequence has at least about 90%, 91%, 92%, 93%, 94%, 95%, 96%, 97%, 98%, 99% or 100% sequence identity with the nucleic acid sequence, e.g., of GenBank accession no. NM-001402.5→NP-001393.1, over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “Microtubule-associated protein Tau” and “MAPT” (also known as DDPAC, FLJ31424, MAPTL, MGC138549, MSTD, MTBT1, MTBT2, PPND and TAU) refer to a protein whose transcript undergoes complex, regulated alternative splicing that leads to a range of different MAPT mRNA transcripts found in neurons based on the maturation state and neuron type. Mutations or deleterious splice variants are associated with neurodegenerative diseases including Alzheimer's disease, Pick's disease, frontotemporal dementia, corticobasal degeneration and progressive supranuclear palsy. Structurally, a MAPT amino acid sequence has at least about 90%, 91%, 92%, 93%, 94%, 95%, 96%, 97%, 98%, 99% or 100% sequence identity with the amino acid sequence, e.g., of GenBank accession no. NM-016835.4→NP-058519.3 (isoform 1); NM-005910.5→NP-005901.2 (isoform 2); NM-016834.4→NP-058518.1 (isoform 3); NM-016841.4→NP-058525.1 (isoform 4); NM-001123067.3→NP-001116539.1 (isoform 5); or NM-001123066.3→NP-001116538.2 (isoform 6), over a sequence length of at least 50, 100, 150, 200, 250, 300, 350 amino acids or over the full length of the polypeptide. Structurally, a MAPT nucleic acid sequence has at least about 90%, 91%, 92%, 93%, 94%, 95%, 96%, 97%, 98%, 99% or 100% sequence identity with the nucleic acid sequence, e.g., of GenBank accession no. NM-016835.4→NP-058519.3 (isoform 1); NM-005910.5→NP-005901.2 (isoform 2); NM-016834.4→NP-058518.1 (isoform 3); NM-016841.4→NP-058525.1 (isoform 4); NM-001123067.3→NP-001116539.1 (isoform 5); or NM-001123066.3→NP-001116538.2 (isoform 6), over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “Dynamin 1-like protein” and “DNM1L” (also known as DLP1, DRP1, DVLP, DYMPLE, HDYNIV and VPS1) refer to a member of the dynamin family of GTPases that plays a role in regulating mitochondrial morphology controlling the distributions of mitochondrial tubules in the cytoplasm. The DNM1L gene produces three alternatively spliced variants which are alternatively polyadenylated. Structurally, a DNM1L amino acid sequence has at least about 90%, 91%, 92%, 93%, 94%, 95%, 96%, 97%, 98%, 99% or 100% sequence identity with the amino acid sequence, e.g., of GenBank accession no. NM-012062.3→NP-036192.2 (isoform 1); NM-012063.2→NP-036193.2 (isoform 2); NM-005690.3→NP-005681.2 (isoform 3), over a sequence length of at least 50, 100, 150, 200, 250, 300, 350 amino acids or over the full length of the polypeptide. Structurally, a DNM1L nucleic acid sequence has at least about 90%, 91%, 92%, 93%, 94%, 95%, 96%, 97%, 98%, 99% or 100% sequence identity with the nucleic acid sequence, e.g., of GenBank accession no. NM-012062.3→NP-036192.2 (isoform 1); NM-012063.2→NP-036193.2 (isoform 2); NM-005690.3→NP-005681.2 (isoform 3), over a sequence length of at least 300, 500, 750, 1000 nucleic acids or over the full length of the polynucleotide. The sequence alignments can be performed using any alignment algorithm known in the art, e.g., BLAST, ALIGN, set to default settings

The terms “Neurofilament, light polypeptide” and “NEFL” (also known as CMT1F, CMT2E, NF-L and NFL) refer to type IV intermediate filament heteropolymers composed of light, medium, and heavy chains. NEFL is a component of the axoskeleton and functions to maintain neuronal morphology and may play a role in intracellular transport to axons and dendrites. Mutations in NEFL cause Charcot-Marie-Tooth diseases types 1F (CMT1F) and 2E (CMT2E)—both peripheral nervous system disorders. NEFL has also been associated with Parkinson disease and Amyotrophic lateral sclerosis (ALS). Structurally, a NEFL amino acid sequence has at least about 90%, 91%, 92%, 93%, 94%, 95%, 96%, 97%, 98%, 99% or 100% sequence identity with the amino acid sequence, e.g., of GenBank accession no. NM-006158.3→NP-006149.2, over a sequence length of at least 50, 100, 150, 200, 250, 300, 350 amino acids or over the full length of the polypeptide. Structurally, a NEFL nucleic acid sequence has at least about 90%, 91%, 92%, 93%, 94%, 95%, 96%, 97%, 98%, 99% or 100% sequence identity with the nucleic acid sequence, e.g., of GenBank accession no. NM-006158.3→NP-006149.2, over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “Radixin” or “RDX” (also known as DFNB24) refers to a cytoskeletal protein involved in linking actin to the plasma membrane. RDX has high sequence identity to Exrin and Moesin. Structurally, a RDX amino acid sequence has at least about 90%, 91%, 92%, 93%, 94%, 95%, 96%, 97%, 98%, 99% or 100% sequence identity with the amino acid sequence, e.g., of GenBank accession no. NM-002906.3→NP-002897.1, over a sequence length of at least 50, 100, 150, 200, 250, 300, 350 amino acids or over the full length of the polypeptide. Structurally, an RDX nucleic acid sequence has at least about 90%, 91%, 92%, 93%, 94%, 95%, 96%, 97%, 98%, 99% or 100% sequence identity with the nucleic acid sequence, e.g., of GenBank accession no. NM-002906.3→NP-002897.1, over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “moesin” and “MSN” and “membrane-organizing extension spike protein” refer to a member of the ERM family which includes ezrin and radixin. ERM proteins function as cross-linkers between plasma membranes and actin-based cytoskeletons. Moesin is localized to filopodia and other membranous protrusions that are important for cell-cell recognition and signaling and for cell movement. Structurally, a MSN amino acid sequence has at least about 90%, 91%, 92%, 93%, 94%, 95%, 96%, 97%, 98%, 99% or 100% sequence identity with the amino acid sequence, e.g., of GenBank accession no. NM-002444.2→NP-002435.1, over a sequence length of at least 50, 100, 150, 200, 250, 300, 350 amino acids or over the full length of the polypeptide. Structurally, an MSN nucleic acid sequence has at least about 90%, 91%, 92%, 93%, 94%, 95%, 96%, 97%, 98%, 99% or 100% sequence identity with the nucleic acid sequence, e.g., of GenBank accession no. NM-002444.2→NP-002435.1, over a sequence length of at least 300, 500, 750, 1000 nucleic acids or over the full length of the polynucleotide. The sequence alignments can be performed using any alignment algorithm known in the art, e.g., BLAST, ALIGN, set to default settings.

The terms “non-specific enolase” and “ENO2” and “NSE” and “gamma enolase” refer to the gene that encodes three enolase enzymes wherein NSE, a homodimer of gamma enolase, is found in neurons and cells of neuronal origin; having neurotrophic and neuroprotective properties on a broad spectrum of central nervous system (CNS) neurons. Structurally, aNSE amino acid sequence has at least about 90%, 91%, 92%, 93%, 94%, 95%, 96%, 97%, 98%, 99% or 100% sequence identity with the amino acid sequence, e.g., of GenBank accession no. NM_001975.3→NP_001966.1, or NCBI Gene ID 2026, over a sequence length of at least 50, 100, 150, 200, 250, 300, 350, 400 amino acids or over the full length of the polypeptide. Structurally, a NSE nucleic acid sequence has at least about 90%, 91%, 92%, 93%, 94%, 95%, 96%, 97%, 98%, 99% or 100% sequence identity with the nucleic acid sequence, e.g., of GenBank accession no. NM_001975.3→NP_001966.1, or NCBI Gene ID 2026, over a sequence length of at least 300, 500, 750, 1000, 1200 nucleic acids or over the full length of the polynucleotide. The sequence alignments can be performed using any alignment algorithm known in the art, e.g., BLAST, ALIGN, set to default settings.

The terms “Caspr2” and “contact in associated protein 2” (also known as CNTNAP2; CDFE; NRXN4; AUTS15; PTHSL1) refer to the gene member of the neurexin family which functions in the vertebrate nervous system as cell adhesion molecules and receptors. Caspr2 comprises epidermal growth factor repeats and laminin G domains, and additionally comprises an F5/8 type C domain, discoidin/neuropilin- and fibrinogen-like domains, thrombospondin N-terminal-like domains and a putative PDZ binding site. Caspr2 is localized at juxtaparanodes of myelinated axons, and can mediate interactions between neurons and glia during nervous system development. Caspr2 can also function in localization of potassium channels within differentiating axons. Structurally, a Caspr2 amino acid sequence has at least about 90%, 91%, 92%, 93%, 94%, 95%, 96%, 97%, 98%, 99% or 100% sequence identity with the amino acid sequence, e.g., of GenBank accession no. NM_014141.6→NP_054860.1 or NCBI Gene ID 26047, over a sequence length of at least 50, 100, 150, 200, 250, 300, 350, 400, 500, 600, 700, 800, 900, 1000, 1100, 1200 amino acids or over the full length of the polypeptide. Structurally, a Caspr2 nucleic acid sequence has at least about 90%, 91%, 92%, 93%, 94%, 95%, 96%, 97%, 98%, 99% or 100% sequence identity with the nucleic acid sequence, e.g., of GenBank accession no. NM_014141.6→NP_054860.1 or NCBI Gene ID 26047, over a sequence length of at least 300, 500, 750, 1000, 1500, 2000, 2500, 3000 nucleic acids or over the full length of the polynucleotide. The sequence alignments can be performed using any alignment algorithm known in the art, e.g., BLAST, ALIGN, set to default settings.

The terms “KCNAB2” and “potassium voltage-gated channel subfamily A regulatory beta subunit 2” and “voltage-gated potassium channel subunit beta-2” (also known as: AKR6A5; KCNA2B; HKvbeta2; KV-BETA-2; HKvbeta2.1; HKvbeta2.2) refer to a member of the potassium channel, voltage-gated, shaker-related subfamily. KCNAB2 is one of the beta subunits, which are auxiliary proteins associating with functional Kv-alpha subunits. KCNAB2 alters functional properties of the KCNA4 gene product. Alternative splicing of this gene results in multiple transcript variants encoding distinct isoforms. Structurally, aKCNAB2 amino acid sequence has at least about 90%, 91%, 92%, 93%, 94%, 95%, 96%, 97%, 98%, 99% or 100% sequence identity with the amino acid sequence, e.g., of GenBank accession no. NM_001199860.2→NP_001186789.1, NM_001199861.2→NP_001186790.1, NM_001199862.2→NP_001186791.1, NM_001199863.2→NP_001186792.1, NM_003636.4→NP_003627.1, NM_172130.3→NP_742128.1, or NCBI Gene ID 8514, over a sequence length of at least 50, 100, 150, 200, 250, 300 amino acids or over the full length of the polypeptide. Structurally, a KCNAB2 nucleic acid sequence has at least about 90%, 91%, 92%, 93%, 94%, 95%, 96%, 97%, 98%, 99% or 100% sequence identity with the nucleic acid sequence, e.g., of GenBank accession no. NM_001199860.2→NP_001186789.1, NM_001199861.2→NP_001186790.1, NM_001199862.2→NP_001186791.1, NM_001199863.2→NP_001186792.1, NM_003636.4→NP_003627.1, NM_172130.3→NP_742128.1, or NCBI Gene ID 8514, over a sequence length of at least 300, 500, 750 nucleic acids or over the full length of the polynucleotide. The sequence alignments can be performed using any alignment algorithm known in the art, e.g., BLAST, ALIGN, set to default settings.

The terms “KCNAB1” and “potassium voltage-gated channel subfamily A member regulatory beta subunit 1” (also known as hKvb3; AKR6A3; KCNA1B; Kvb1.3; hKvBeta3; KV-BETA-1) refer to a member of the potassium channel, voltage-gated, shaker-related subfamily. KCNAB1 is a cytoplasmic potassium channel subunit that modulates the characteristics of the channel-forming alpha-subunits. Structurally, a KCNAB1 amino acid sequence has at least about 90%, 91%, 92%, 93%, 94%, 95%, 96%, 97%, 98%, 99% or 100% sequence identity with the amino acid sequence, e.g., of GenBank accession no. NM_001308217.1→NP_001295146.1, NM_001308222.1→NP_001295151.1, NM_003471.3→NP_003462.2, NM_172159.3→NP_751891.1, NM_172160.2→NP_751892.1, or NCBI Gene ID 7881, over a sequence length of at least 50, 100, 150, 200, 250, 300, 350, 400 amino acids or over the full length of the polypeptide. Structurally, a KCNAB1 nucleic acid sequence has at least about 90%, 91%, 92%, 93%, 94%, 95%, 96%, 97%, 98%, 99% or 100% sequence identity with the nucleic acid sequence, e.g., of GenBank accession no. NM_001308217.1→NP_001295146.1, NM_001308222.1→NP_001295151.1, NM_003471.3→NP_003462.2, NM_172159.3→NP_751891.1, NM_172160.2→NP_751892.1, or NCBI Gene ID 7881, over a sequence length of at least 300, 500, 750, 1000, 1200 nucleic acids or over the full length of the polynucleotide. The sequence alignments can be performed using any alignment algorithm known in the art, e.g., BLAST, ALIGN, set to default settings.

The terms “endothelial integrin ligand” and “EDIL3” and “EGF like repeats and discoidin domains 3” and “DEL1” refer to an integrin ligand. EDIL3 can function to promote adhesion of endothelial cells through interaction with the alpha-v/beta-3 integrin receptor and can function in the regulation of vascular morphogenesis of remodeling in embryonic development. Structurally, a EDIL3 amino acid sequence has at least ab out 90%, 91%, 92%, 93%, 94%, 95%, 96%, 97%, 98%, 99% or 100% sequence identity with the amino acid sequence, e.g., of GenBank accession no. NM_001278642.1→NP_001265571.1, NM_005711.5→NP_005702.3, or NCBI Gene ID 10085 over a sequence length of at least 50, 100, 150, 200, 250, 300, 350, 400, 450 amino acids or over the full length of the polypeptide. Structurally, a EDTL3 nucleic acid sequence has at least about 90%, 91%, 92%, 93%, 94%, 95%, 96%, 97%, 98%, 99% or 100% sequence identity with the nucleic acid sequence, e.g., of GenBank accession no. NM_001278642.1→NP_001265571.1, NM_005711.5→NP_005702.3, or NCBI Gene ID 10085, over a sequence length of at least 300, 500, 750, 1000, 1200 nucleic acids or over the full length of the polynucleotide. The sequence alignments can be performed using any alignment algorithm known in the art, e.g., BLAST, ALIGN, set to default settings.

The terms “IVD” and “isovaleryl-CoA dehydrogenase” and “ACAD2” refer to a mitochondrial matrix enzyme that catalyzes the third step in leucine catabolism. Structurally, a IVD amino acid sequence has at least about 90%, 91%, 92%, 93%, 94%, 95%, 96%, 97%, 98%, 99% or 100% sequence identity with the amino acid sequence, e.g., of GenBank accession no. NM_001159508.3→NP_001152980.2, NM_001354597.3→NP_001341526.1, NM_001354598.3→NP_001341527.2, NM_001354599.3→NP_001341528.2, NM_001354600.3→NP_001341529.2, NM_001354601.3→NP_001341530.2, NM_002225.5→NP_002216.3, or NCBI Gene ID 3712, over a sequence length of at least 50, 100, 150, 200, 250, 300, 350, 400, 450 amino acids or over the full length of the polypeptide. Structurally, a IVD nucleic acid sequence has at least about 90%, 91%, 92%, 93%, 94%, 95%, 96%, 97%, 98%, 99% or 100% sequence identity with the nucleic acid sequence, e.g., of GenBank accession no. NM_001159508.3→NP_001152980.2, NM_001354597.3→NP_001341526.1, NM_001354598.3→NP_001341527.2, NM_001354599.3→NP_001341528.2, NM_001354600.3→NP_001341529.2, NM_001354601.3→NP_001341530.2, NM_002225.5→NP_002216.3, or NCBI Gene ID 3712, over a sequence length of at least 300, 500, 750, 1000, 1200 nucleic acids or over the full length of the polynucleotide. The sequence alignments can be performed using any alignment algorithm known in the art, e.g., BLAST, ALIGN, set to default settings.

The terms “bran specific sultransferase” and “SULT4A1” and “sulfotransferase family 4A member 1” (also known as NST; BRSTL1; SULTX3; BR-STL-1; DJ388M5.3; hBR-STL-1) refer to a member of the sulfotransferase family. SULT4A1 is a brain-specific sulfotransferase that can function in the metabolism of neurotransmitters. Structurally, a SULT4A1 amino acid sequence has at least about 90%, 91%, 92%, 93%, 94%, 95%, 96%, 97%, 98%, 99% or 100% sequence identity with the amino acid sequence, e.g., of GenBank accession no. XM_011530121.1→XP_011528423.1, XM_024452212.1→XP 024307980.1, XM_011530120.3→XP_011528422.1, or NCBI Gene ID 25830, over a sequence length of at least 50, 100, 150, 200, 250 amino acids or over the full length of the polypeptide. Structurally, a SULT4A1 nucleic acid sequence has at least about 90%, 91%, 92%, 93%, 94%, 95%, 96%, 97%, 98%, 99% or 100% sequence identity with the nucleic acid sequence, e.g., of GenBank accession no. XM_011530121.1→XP_011528423.1, XM_024452212.1→XP_024307980.1, XM_011530120.3→XP_011528422.1, or NCBI Gene ID 25830, over a sequence length of at least 300, 500, 750 nucleic acids or over the full length of the polynucleotide. The sequence alignments can be performed using any alignment algorithm known in the art, e.g., BLAST, ALIGN, set to default settings.

The terms “TNIP2” and “TNFAIP3 interacting protein 2” (also known as KLIP; ABIN2; FLIP1) refer to an inhibitor of NFkappaB activation. TNIP2 can also function in MAP/ERK signaling pathway in the innate immune response and in the regulation of apoptosis in endothelial cells. Structurally, a TNIP2 amino acid sequence has at least about 90%, 91%, 92%, 93%, 94%, 95%, 96%, 97%, 98%, 99% or 100% sequence identity with the amino acid sequence, e.g., of GenBank accession no. NM_001161527.2→NP_001154999.1, NM_001292016.2→NP_001278945.1, NM_024309.4→NP_077285.3, or NCBI Gene ID 79155, over a sequence length of at least 50, 100, 150, 200, 250, 300 amino acids or over the full length of the polypeptide. Structurally, a TNIP2 nucleic acid sequence has at least about 90%, 91%, 92%, 93%, 94%, 95%, 96%, 97%, 98%, 99% or 100% sequence identity with the nucleic acid sequence, e.g., of GenBank accession no. NM_001161527.2→NP_001154999.1, NM_001292016.2→NP_001278945.1, NM_024309.4→NP_077285.3, or NCBI Gene ID 79155, over a sequence length of at least 300, 500, 750, 900 nucleic acids or over the full length of the polynucleotide. The sequence alignments can be performed using any alignment algorithm known in the art, e.g., BLAST, ALIGN, set to default settings.

The terms “retinoic acid induced 16” and “RAI16” and “family with sequence similarity 160 member B2” refer to a gene expressed in neural tissue. Structurally, a RAI16 amino acid sequence has at least about 90%, 91%, 92%, 93%, 94%, 95%, 96%, 97%, 98%, 99% or 100% sequence identity with the amino acid sequence, e.g., of GenBank accession no NM_001354250.2→NP_001341179.1, NM_001354251.2→NP_001341180.1, NM_022749.7→NP_073586.5, or NCBI Gene ID 64760, over a sequence length of at least 50, 100, 150, 200, 250, 300, 400, 500, 600, 700 amino acids or over the full length of the polypeptide. Structurally, a RAI16 nucleic acid sequence has at least about 90%, 91%, 92%, 93%, 94%, 95%, 96%, 97%, 98%, 99% or 100% sequence identity with the nucleic acid sequence, e.g., of GenBank accession no. NM_001354250.2→NP_001341179.1, NM_001354251.2→NP_001341180.1, NM_022749.7→NP_073586.5, or NCBI Gene ID 64760, over a sequence length of at least 300, 500, 750, 900, 1200, 1500, 1800, 2100 nucleic acids or over the full length of the polynucleotide. The sequence alignments can be performed using any alignment algorithm known in the art, e.g., BLAST, ALIGN, set to default settings.

The terms “GDP D6” and “glycerophosphocholine phosphodiesterase 1” (also known as GPCD1, EDI3; GDE5; GDPD6; PREI4) refer to a glycerophosphocholine phosphodiesterase. Structurally, a GDP D6 amino acid sequence has at least about 90%, 91%, 92%, 93%, 94%, 95%, 96%, 97%, 98%, 99% or 100% sequence identity with the amino acid sequence, e.g., of GenBank accession no. NM_019593.5→NP_062539.1, or NCBI Gene ID 56261, over a sequence length of at least 50, 100, 150, 200, 250, 300, 400, 500, 600 amino acids or over the full length of the polypeptide. Structurally, a GDP D6 nucleic acid sequence has at least about 90%, 91%, 92%, 93%, 94%, 95%, 96%, 97%, 98%, 99% or 100% sequence identity with the nucleic acid sequence, e.g., of GenBank accession no. NM_019593.5→NP_062539.1, or NCBI Gene ID 56261, over a sequence length of at least 300, 500, 750, 900, 1200, 1500, 1800 nucleic acids or over the full length of the polynucleotide. The sequence alignments can be performed using any alignment algorithm known in the art, e.g., BLAST, ALIGN, set to default settings.

The terms “ezrin” and “EZR” (also known as CVL; CVIL; VIL2; MGC1584; FLJ26216; DKFZp762H157) refer to a cytoplasmic peripheral membrane protein that functions as a protein-tyrosine kinase substrate in microvilli. As a member of the ERM protein family, this protein serves as an intermediate between the plasma membrane and the actin cytoskeleton. EZR protein plays a role in cell surface structure adhesion, migration and organization, and has been implicated in various human cancers. Structurally, a EZR amino acid sequence has at least about 90%, 91%, 92%, 93%, 94%, 95%, 96%, 97%, 98%, 99% or 100% sequence identity with the amino acid sequence, e.g., of GenBank accession no. NM-003379.4→NP-003370.2 (isoform 1) or NM-001111077.1→NP-001104547.1 (isoform 2), over a sequence length of at least 50, 100, 150, 200, 250, 300, 350 amino acids or over the full length of the polypeptide. Structurally, a EZR nucleic acid sequence has at least about 90%, 91%, 92%, 93%, 94%, 95%, 96%, 97%, 98%, 99% or 100% sequence identity with the nucleic acid sequence, e.g., of GenBank accession no. NM-003379.4→NP-003370.2 (isoform 1) or NM-001111077.1→NP-001104547.1 (isoform 2), over a sequence length of at least 300, 500, 750, 1000 nucleic acids or over the full length of the polynucleotide. The sequence alignments can be performed using any alignment algorithm known in the art, e.g., BLAST, ALIGN, set to default settings.

In some embodiments, the detection of the presence of auto-antibodies are performed prior to conception. In some embodiments, the detection of auto-antibodies are performed upon confirming pregnancy. In some embodiments, the detection of auto-antibodies are performed after confirming pregnancy. In some embodiments, detection is performed weekly during pregnancy. In some embodiments, detection is performed monthly. In some embodiments, detection is performed at least once during each trimester. In some embodiments, the detection of auto-reactive antibodies is performed after birth.

### Antibodies

Methods as disclosed herein for inhibiting of FcRn function within the pregnant individual, individual trying to become pregnant, or considering pregnancy are characterized by the administration of an FcRn-binding antibody or a target-binding fragment thereof.

Methods, as disclosed herein, for reducing auto-reactive antibodies (e.g., an amount present in blood or sera) capable of contacting an embryo and/or fetus of a pregnant individual, an individual trying to become pregnant, an individual considering pregnancy, or an individual serving as a surrogate for the pregnancy, are characterized by the inhibition of FcRn function by an antibody, or fragment thereof, within the pregnant individual, individual trying to become pregnant, or considering pregnancy. Such antibodies, as disclosed, can also be used for reducing auto-reactive antibodies by preventing an interaction between an IgG molecule and FcRn in a pregnant individual, an individual trying to become pregnant, an individual considering pregnancy, or an individual serving as a surrogate for the pregnancy. In some embodiments, an antibody inhibiting FcRn function comprises an antibody that inhibits the transfer of maternal antibodies across a placental barrier. In certain embodiments, an antibody inhibiting FcRn function comprises an antibody that inhibits the transfer of maternal antibodies to a region wherein the antibodies can contact an embryo or fetus. In certain embodiments, an antibody inhibiting FcRn function comprises an antibody that inhibits FcRn-mediated IgG transfer across or through endosomal vesicles in the syncytiotrophoblast of the placenta. In some embodiments, an antibody inhibiting FcRn function comprises an antibody that inhibits or block the interaction between FcRn and an antibody molecule. In some embodiments, an antibody inhibiting FcRn function comprises an antibody that inhibits, blocks, and/or reduces FcRn-mediated recycling or rescue of an antibody molecule. In some embodiments, an antibody inhibiting FcRn function comprises an antibody that inhibits accessory molecules involved in FcRn function.

Among the provided antibodies are monoclonal antibodies, polyclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A polyclonal antibody is a preparation that includes different antibodies of varying sequences that generally are directed against two or more different determinants (epitopes). The monoclonal antibody can comprise a human IgG1 constant region. The monoclonal antibody can comprise a human IgG4 constant region.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)).

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, antibody variants having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved ADCC or CDC.

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots”. In some embodiments of the variant VH and VL sequences, each CDR is unaltered.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37(2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, the antibodies are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn297 of the CH2 domain of the Fc region (See e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennar oligosaccharide structure. Modifications of the oligosaccharide in an antibody can be made, for example, to create antibody variants with certain improved properties. Antibody glycosylation variants can have improved ADCC and/or CDC function. In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (See e.g., WO 08/077546). Asn297 refers to the asparagine residue located at about position 297 in the Fc region (EU numbering of Fc region residues; See e.g., Edelman et al. Proc Natl Acad Sci USA. 1969 May; 63(1):78-85). However, Asn297 may also be located about ±3 amino acids upstream or downstream of position 297, i.e., between positions 294 and 300, due to minor sequence variations in antibodies. Such fucosylation variants can have improved ADCC function (See e.g., Okazaki et al. J. Mol. Biol. 336:1239-1249 (2004); and Yamane-Ohnuki et al. Biotech. Bioeng. 87: 614 (2004)). Cell lines, e.g., knockout cell lines and methods of their use can be used to produce defucosylated antibodies, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87: 614 (2004); Kanda, Y. et al., Biotechnol. Bioeng., 94(4):680-688 (2006)). Other antibody glycosylation variants are also included (See e.g., U.S. Pat. No. 6,602,684).

In some embodiments, an antibody provided herein has a dissociation constant (KD) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or less (e.g., 10-8 M or less, e.g., from 10-8 M to 10-13 M, e.g., from 10-9 M to 10-13 M) for the antibody target. The antibody target can be an [Insert] target. KD can be measured by any suitable assay. In certain embodiments, KD can be measured using surface plasmon resonance assays (e.g., using a BIACORE®-2000 or a BIACORE®-3000, or an Octet).

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcTR binding (hence likely lacking ADCC activity) but retains FcRn binding ability. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays).

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A118 (EU numbering) of the heavy chain; and S400 (EU numbering) of the heavy chain Fc region.

In some embodiments, an antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylene oxide/ethylene oxide copolymers, poly oxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The terms “homologous” and “homology” and “percent homology” when used herein to describe an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

Known FcRn antibodies, used in non-ASD disease models (e.g. autoimmune disorders), can be used for inhibiting FcRn function. In certain embodiments, the antibody, or target-binding fragment thereof, is selected from the group consisting of Rozanolixizumab, SYNT001 (ALXN1830), M281, Argx-113, HL161-11G, HL161-11H, HL161-1A, DX-2504, DX-2507, ABY039,IMVT-1401/RVT1401, and any combination thereof. In certain embodiments, the antibody is Rozanolixizumab (as disclosed in: “Generation and characterization of a high affinity anti-human FcRn antibody, rozanolixizumab, and the effects of different molecular formats on the reduction of plasma IgG concentration” MAbs. 2018 October; 10(7):1111-1130. doi: 10.1080/19420862.2018.1505464. Epub 2018 Sep. 12). In certain embodiments, the antibody is SYNT001 (as disclosed in: “Blocking FcRn in humans reduces circulating IgG levels and inhibits IgG immune complex-mediated immune responses” Sci Adv. 2019 Dec. 18; 5(12):eaax9586. doi: 10.1126/sciadv.aax9586. eCollection 2019 December). In certain embodiments, the antibody M281 (as disclosed in: “M281, an Anti-FcRn Antibody: Pharmacodynamics, Pharmacokinetics, and Safety Across the Full Range of IgG Reduction in a First-in-Human Study” Clin Pharmacol Ther. 2019 April; 105(4):1031-1039. doi: 10.1002/cpt.1276. Epub 2018 Dec. 4). In certain embodiments, the antibody is Argx-113 (as disclosed in: “Randomized phase 2 study of FcRn antagonist efgartigimod in generalized myasthenia gravis” Neurology. 2019 Jun. 4; 92(23):e2661-e2673. Epub 2019 May 22.). In certain embodiments, the antibody is HL161-11G (U.S. Pat. No. 10,280,207). In certain embodiments, the antibody is HL161-1A (U.S. Pat. No. 10,280,207). In certain embodiments, the antibody is HL161-11H (U.S. Pat. No. 10,280,207). In certain embodiments, the antibody is DX-2504 (as disclosed in: “Fully human monoclonal antibody inhibitors of the neonatal fc receptor reduce circulating IgG in non-human primates” Front Immunol. 2015 Apr. 23; 6:176. doi: 10.3389/fimmu.2015.00176. eCollection 2015). In certain embodiments, the antibody is DX-2507 (as disclosed in: “Fully human monoclonal antibody inhibitors of the neonatal fc receptor reduce circulating IgG in non-human primates” Front Immunol. 2015 Apr. 23; 6:176. doi: 10.3389/fimmu.2015.00176. eCollection 2015). In certain embodiments, the antibody is ABY039,IMVT-1401/RVT1401. In certain embodiments, the antibody or the target-binding fragment thereof comprises a complementarity-determining region of an antibody selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof.

The antibodies described herein can be derived from known FcRn antibodies and can comprise a heavy and/or light chain variable region at least about 90%, 95%, 97%, 98%, 99%, or 100% identical to Rozanolixizumab, SYNT001, M281, Argx-113, HL161-11G, HL161-11H, HL161-1A, DX-2504, DX-2507, or ABY039,IMVT-1401/RVT1401. Any of these antibodies can be formatted on a constant region, such as IgG4 that has reduced effector function, or that comprises one or more mutations or modifications to reduce effector function. The antibodies and antibody fragments, including variable regions and CDR regions, described herein may suitably be formatted as part of an antibody with reduced effector function. In certain embodiments, the antibody may be a F(ab′)₂, which lacks the Fc region. In certain embodiments, the antibody may comprise one or more mutations to the constant region of an antibody heavy chain that reduces effector function, such as antibody dependent cellular cytotoxicity or complement dependent cytotoxicity. In certain embodiments, the antibody may comprise an IgG4 constant region. In certain embodiments, the one or more mutations to reduce one or more effector functions of the recombinant antibody or antigen binding fragment thereof comprise a mutation or set of mutations selected from: N434A, N434H, T307A/E380A/N434A, M252Y/S254T/T256E, 433K/434F/436H, T250Q, T250F, M428L, M428F, T250Q/M428L, N434S, V308W, V308Y, V308F, M252Y/M428L, D259I/V308F, M428L/V308F, Q311V/N434S, T307Q/N434A, E258F/V427T, S228P, L235E, S228P/L235E/R409K, S228P/L235E, K370Q, K370E, deletion of G446, deletion of K447, and any combination thereof of IgG4 according to the EU numbering system. In certain embodiments, the antibody may comprise an IgG4 constant region which has a mutation corresponding to S228P of the heavy chain according to the EU numbering system. In certain embodiments, the antibody may comprise an IgG4PAA constant region, which has mutations corresponding to S228P, F234A, and L235A of the heavy chain according to the EU numbering system. See Parekh et al. “Development and validation of an antibody-dependent cell-mediated cytotoxicity-reporter gene assay.” MAbs 2012 May 1; 4(3): 310-318.

In some embodiments, the antibody (e.g., an antibody that inhibits FcRn function) is an Fc-engineered antibody that enhances IgG degradation. In certain instances, the antibody is an antibody engineered to bind with increased affinity to FcRn through their Fc region at both acidic and near neutral pH. In such instances, the altered (e.g., enhanced) FcRN binding promotes degradation of a target antibody (e.g., a maternal auto-antibody). In some embodiments, the antibody (e.g., an antibody that inhibits FcRn function) comprises a variable domain that recognizes the Fc region of an antibody and a variant Fc region, or FcRn-binding fragment thereof, wherein the Fe domains of the Fc region or FcRn-binding fragment thereof comprise the one or more amino acid substitutions that increase binding FcRn (e.g., as described herein) relative to a wild-type IgG1 Fc region. In some embodiments, the one or more amino acid substitutions comprises Y, T, E, K, F, and Y at EU positions 252, 254, 256, 433, 434, and 436 respectively, and wherein the Fc region binds to FcRn with increased affinity and reduced pH dependence relative to a wild-type IgG1 Fc region. Such antibodies and variant Fc also exemplified in U.S. Pat. No. 10,316,073 and Vaccaro C, Zhou J, Ober R J, Ward E S. Engineering the Fc region of immunoglobulin G to modulate in vivo antibody levels. Nat Biotechnology. 2005 October; 23(10):1283-8. doi: 10.1038/nbt1143.

The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. Therapeutically effective amounts include amounts are those sufficient to ameliorate one or more symptoms associated with the disease or affliction to be treated.

### Small Molecules

Methods as disclosed herein for inhibiting of FcRn function within the pregnant individual, individual trying to become pregnant, or considering pregnancy are characterized by the administration of an FcRn small molecule drug inhibitor.

A small-molecule drug can be any article, chemical, or biological entity of low molecular weight intended to play a role in disease, or otherwise affect a structure or function in human or animal. Likewise, a small molecule drug can be any organic compound that affects a biologic process and comprises with a relatively low molecular weight such as those with a molecular weight of 5 kDa or below.

Methods, as disclosed herein, for reducing auto-reactive antibodies capable of contacting an embryo and/or fetus of a pregnant individual, an individual trying to become pregnant, an individual considering pregnancy, or an individual serving as a surrogate for the pregnancy, are characterized by the inhibition of FcRn function by a small molecule within the pregnant individual, individual trying to become pregnant, or considering pregnancy. Such small molecules, as disclosed, can also be used for reducing auto-reactive antibodies by preventing an interaction between an IgG molecule and FcRn in a pregnant individual, an individual trying to become pregnant, an individual considering pregnancy, or an individual serving as a surrogate for the pregnancy. In some embodiments, a small molecule inhibiting FcRn function comprises a small molecule that inhibits the transfer of maternal antibodies across a placental barrier. In certain embodiments, a small molecule inhibiting FcRn function comprises a small molecule that inhibits the transfer of maternal antibodies to a region wherein the maternal antibodies can contact an embryo or fetus. In certain embodiments, a small molecule inhibiting FcRn function comprises a small molecule that inhibits FcRn-mediated IgG transfer across or through endosomal vesicles in the syncytiotrophoblast of the placenta. In some embodiments, a small molecule inhibiting FcRn function comprises a small molecule that inhibits or block the interaction between FcRn and a small molecule. In some embodiments, a small molecule inhibiting FcRn function comprises a small molecule that inhibits, blocks, and/or reduces FcRn-mediated recycling or rescue of a small molecule. In some embodiments, a small molecule inhibiting FcRn function comprises a small molecule that inhibits accessory molecules involved in FcRn function.

Methods for screening and identifying small molecules that bind and/or inhibit FcRn function can be conceived to one skilled in the art. In vitro inhibition of an interaction between FcRn and an IgG Fc molecule, or fragment thereof, can be expected to inhibit FcRn function in vivo. Exemplary methods, for example, are disclosed in Wang Z, Fraley C, Mezo A R. Discovery and structure-activity relationships of small molecules that block the human immunoglobulin G-human neonatal Fc receptor (hIgG-hFcRn) protein-protein interaction. Bioorg Med Chem Lett. 2013 Mar. 1; 23(5):1253-6. doi: 10.1016/j.bmcl.2013.01.014 or Grevys, A., Nilsen, J., Sand, K. M. K. et al. A human endothelial cell-based recycling assay for screening of FcRn targeted molecules. Nat Commun 9, 621(2018) or Vaccaro C, Zhou J, Ober R J, Ward E S. Engineering the Fc region of immunoglobulin G to modulate in vivo antibody levels. Nat Biotechnol. 2005 October; 23(10):1283-8. doi: 10.1038/nbt1143. In certain instances, competition inhibition about equal to or greater than a Abdeg (e.g., the Abdeg provided in the Examples).

The binding of a small molecule to FcRn can be sufficient to inhibit binding of an IgG molecule to the FcRn. In certain embodiments, the binding affinity of a small molecule to FcRn is about 1 nm to about 500 nm. In certain embodiments, the binding affinity of a small molecule to FcRn is about 1 nm to about 10 nm, about 1 nm to about 50 nm, about 1 nm to about 100 nm, about 1 nm to about 500 nm, about 1 nm to about 1 nm, about 10 nm to about 50 nm, about 10 nm to about 100 nm, about 10 nm to about 500 nm, about 10 nm to about 1 nm, about 50 nm to about 100 nm, about 50 nm to about 500 nm, about 50 nm to about 1 nm, about 100 nm to about 500 nm, about 100 nm to about 1 nm, or about 500 nm to about 1 nm. In certain embodiments, the binding affinity of a small molecule to FcRn is about 1 nm, about 10 nm, about 50 nm, about 100 nm, about 500 nm, or about 1 nm. In certain embodiments, the binding affinity of a small molecule to FcRn is at least about 1 nm, about 10 nm, about 50 nm, about 100 nm, or about 500 nm. In certain embodiments, the binding affinity of a small molecule to FcRn is at most about 10 nm, about 50 nm, about 100 nm, about 500 nm, or about 1 nm.

The small molecule can comprise half-maximal inhibitory concentration (IC50), derived from a competition assay, sufficient to inhibit FcRn function. In certain embodiments, the IC50 value of a small molecule inhibitor of FcRn binding to an IgG is about 1 nm to about 1,000,000 nm. In certain embodiments, the IC50 value of a small molecule inhibitor of FcRn binding to an IgG is about 1 nm to about 10 nm, about 1 nm to about 100 nm, about 1 nm to about 250 nm, about 1 nm to about 500 nm, about 1 nm to about 750 nm, about 1 nm to about 1,000 nm, about 1 nm to about 5,000 nm, about 1 nm to about 10,000 nm, about 1 nm to about 1,000,000 nm, about 10 nm to about 100 nm, about 10 nm to about 250 nm, about 10 nm to about 500 nm, about 10 nm to about 750 nm, about 10 nm to about 1,000 nm, about 10 nm to about 5,000 nm, about 10 nm to about 10,000 nm, about 10 nm to about 1,000,000 nm, about 100 nm to about 250 nm, about 100 nm to about 500 nm, about 100 nm to about 750 nm, about 100 nm to about 1,000 nm, about 100 nm to about 5,000 nm, about 100 nm to about 10,000 nm, about 100 nm to about 1,000,000 nm, about 250 nm to about 500 nm, about 250 nm to about 750 nm, about 250 nm to about 1,000 nm, about 250 nm to about 5,000 nm, about 250 nm to about 10,000 nm, about 250 nm to about 1,000,000 nm, about 500 nm to about 750 nm, about 500 nm to about 1,000 nm, about 500 nm to about 5,000 nm, about 500 nm to about 10,000 nm, about 500 nm to about 1,000,000 nm, about 750 nm to about 1,000 nm, about 750 nm to about 5,000 nm, about 750 nm to about 10,000 nm, about 750 nm to about 1,000,000 nm, about 1,000 nm to about 5,000 nm, about 1,000 nm to about 10,000 nm, about 1,000 nm to about 1,000,000 nm, about 5,000 nm to about 10,000 nm, about 5,000 nm to about 1,000,000 nm, or about 10,000 nm to about 1,000,000 nm. In certain embodiments, the IC50 value of a small molecule inhibitor of FcRn binding to an IgG is about 1 nm, about 10 nm, about 100 nm, about 250 nm, about 500 nm, about 750 nm, about 1,000 nm, about 5,000 nm, about 10,000 nm, or about 1,000,000 nm. In certain embodiments, the IC50 value of a small molecule inhibitor of FcRn binding to an IgG is at least about 1 nm, about 10 nm, about 100 nm, about 250 nm, about 500 nm, about 750 nm, about 1,000 nm, about 5,000 nm, or about 10,000 nm. In certain embodiments, the C50 value of a small molecule inhibitor of FcRn binding to an IgG is at most about 10 nm, about 1 nm, about 250 nm, about 500 nm, about 750 nm, about 1,000 nm, about 5,000 nm, about 10,000 nm, or about 1,000,000 nm. 01261 In some embodiments, the small molecule is selected from TABLE 1.

TABLE 1
FcRn Small Molecule Inhibitors in FcRn Competition Assay

| | | Standard | Standard | Standard | Standard |
|---|---|---|---|---|---|
| PUBCHEM_SID | PUBCHEM_CID | Type | Relation | Value | Units |
| 164139900 | 71561050 | IC50 | = | 2000 | nM |
| 164145501 | 71560669 | IC50 | = | 6000 | nM |
| 164134304 | 71560032 | IC50 | = | 10000 | nM |
| 164145471 | 71560289 | IC50 | = | 30000 | nM |
| 164128585 | 71560923 | IC50 | = | 40000 | nM |
| 164134305 | 71560034 | IC50 | = | 40000 | nM |
| 164137122 | 71560792 | IC50 | = | 40000 | nM |
| 164142693 | 71561049 | IC50 | = | 40000 | nM |
| 164128624 | 3254660 | IC50 | = | 50000 | nM |
| 164139932 | 71559910 | IC50 | = | 80000 | nM |
| 164128586 | 71560286 | IC50 | = | 100000 | nM |
| 164148318 | 71559909 | IC50 | = | 100000 | nM |
| 164148319 | 71560165 | IC50 | = | 100000 | nM |
| 164144478 | 659475 | IC50 | = | 120000 | nM |
| 164148320 | 4323282 | IC50 | = | 120000 | nM |
| 164134303 | 71559908 | IC50 | = | 150000 | nM |
| 164142727 | 71560036 | IC50 | = | 150000 | nM |
| 164142731 | 3332968 | IC50 | > | 150000 | nM |
| 164142692 | 71560919 | IC50 | = | 200000 | nM |
| 164148284 | 71560921 | IC50 | = | 200000 | nM |
| 164139933 | 71560164 | IC50 | = | 250000 | nM |
| 164145503 | 71559771 | IC50 | > | 250000 | nM |
| 164142724 | 71560417 | IC50 | = | 400000 | nM |
| 164145470 | 71560920 | IC50 | = | 400000 | nM |
| 164128587 | 71560287 | IC50 | = | 500000 | nM |
| 164128625 | 71560033 | IC50 | > | 500000 | nM |
| 164128626 | 71560166 | IC50 | > | 500000 | nM |
| 164131448 | 71560789 | IC50 | > | 500000 | nM |
| 164131449 | 71560416 | IC50 | > | 500000 | nM |
| 164131475 | 71560035 | IC50 | > | 500000 | nM |
| 164134259 | 71560671 | IC50 | > | 500000 | nM |
| 164134260 | 71560672 | IC50 | > | 500000 | nM |
| 164134261 | 71560922 | IC50 | > | 500000 | nM |
| 164134262 | 71560414 | IC50 | > | 500000 | nM |
| 164134301 | 71560543 | IC50 | > | 500000 | nM |
| 164134302 | 71560545 | IC50 | > | 500000 | nM |
| 164137123 | 71560415 | IC50 | > | 500000 | nM |
| 164137168 | 3764758 | IC50 | > | 500000 | nM |
| 164139901 | 71560288 | IC50 | > | 500000 | nM |
| 164139931 | 71560668 | IC50 | > | 500000 | nM |
| 164142691 | 71560790 | IC50 | > | 500000 | nM |
| 164142725 | 71560546 | IC50 | > | 500000 | nM |
| 164142726 | 71560547 | IC50 | > | 500000 | nM |
| 164142728 | 71719292 | IC50 | > | 500000 | nM |
| 164145468 | 71560673 | IC50 | > | 500000 | nM |
| 164145469 | 71560791 | IC50 | > | 500000 | nM |
| 164145499 | 71560418 | IC50 | > | 500000 | nM |
| 164145500 | 71560544 | IC50 | > | 500000 | nM |
| 164145504 | 71559772 | IC50 | > | 500000 | nM |
| 164148283 | 71720490 | IC50 | > | 500000 | nM |
| 164148317 | 71560670 | IC50 | > | 500000 | nM |
| 164148321 | 3742943 | IC50 | > | 500000 | nM |

### Peptides

Methods as disclosed herein for inhibiting of FcRn function within the pregnant individual, individual trying to become pregnant, or considering pregnancy are characterized by the administration of an FcRn inhibitor, wherein the FcRn inhibitor is a peptide or polypeptide.

“Polypeptide” and “protein” are used interchangeably herein and include a molecular chain of two or more amino acids linked covalently through peptide bonds. The terms do not refer to a specific length of the product. Thus, “peptides,” and “oligopeptides,” are included within the definition of polypeptide. The term “peptide analog” refers to a peptide having a sequence that differs from a peptide sequence existing in nature by at least one amino acid residue substitution, internal addition, or internal deletion of at least one amino acid, and/or amino- or carboxy-terminal end truncations or additions, and/or carboxy-terminal amidation. An “internal deletion” refers to absence of an amino acid from a sequence existing in nature at a position other than the N- or C-terminus. Likewise, an “internal addition” refers to presence of an amino acid in a sequence existing in nature at a position other than the N- or C-terminus.

A mimetic peptide or peptide mimetic can refer to a peptide or protein having biological activity comparable to a naturally occurring protein of interest, for example, but not limited to, a toxin peptide molecule, e.g., naturally occurring IgG or Fc peptide. These terms further include peptides that indirectly mimic the activity of a naturally occurring peptide molecule, such as by potentiating the effects of the naturally occurring molecule.

The term antagonist peptide, peptide antagonist, and inhibitor peptide can be a peptide that blocks or in some way interferes with the biological activity the FcRn receptor, or has biological activity comparable to a known antagonist or inhibitor of a receptor of interest, such as, but not limited to, an antibody that bind FcRn and inhibits binding of an IgG Fc molecule.

An FcRn inhibitor protein molecule or a domain thereof. A domain of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain). A fusion protein is a protein that includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein. Exemplary fusion polypeptides (e.g., proteins) are exemplified in US PGPUB US20200031948 and in Devanaboyina, S., Khare, P., Challa, D. et al. Engineered clearing agents for the selective depletion of antigen-specific antibodies. Nat Commun 8, 15314 (2017). https://doi.org/10.1038/ncomms15314

A peptide or protein can be a recombinant molecule, wherein recombinant indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. A “recombinant DNA molecule,” is comprised of segments of DNA

A peptide inhibiting FcRn function can comprise a peptide that inhibits the transfer of maternal antibodies across a placental barrier. In certain embodiments, inhibiting FcRn function comprises a peptide that inhibits transfer of maternal antibodies to a region wherein the maternal antibodies can contact an embryo or fetus. In certain embodiments, a peptide inhibiting FcRn function comprises a peptide that inhibits FcRn-mediated IgG transfer across or through endosomal vesicles in the syncytiotrophoblast of the placenta. In some embodiments, a peptide inhibiting FcRn function comprises a peptide that inhibits or blocks the interaction between FcRn and an antibody molecule. In some embodiments, a peptide inhibiting FcRn function comprises a peptide that inhibits, blocks, and/or reduces FcRn-mediated recycling or rescue of an antibody molecule. In some embodiments, a peptide inhibiting FcRn function comprises a peptide that inhibits accessory molecules involved in FcRn function.

A peptide inhibiting FcRn function can comprise a polypeptide that inhibits the transfer of maternal antibodies across a placental barrier. In certain embodiments, inhibiting FcRn function comprises a polypeptide that inhibits transfer of maternal antibodies to a region wherein the maternal antibodies can contact an embryo or fetus. In certain embodiments, a polypeptide inhibiting FcRn function comprises a polypeptide that inhibits FcRn-mediated IgG transfer across or through endosomal vesicles in the syncytiotrophoblast of the placenta. In some embodiments, a polypeptide inhibiting FcRn function comprises a polypeptide that inhibits or blocks the interaction between FcRn and an antibody molecule. In some embodiments, a polypeptide inhibiting FcRn function comprises a polypeptide that inhibits, blocks, and/or reduces FcRn-mediated recycling or rescue of an antibody molecule. In some embodiments, a polypeptide inhibiting FcRn function comprises a peptide that inhibits accessory molecules involved in FcRn function. In some embodiments, the polypeptide comprises a variant Fc region, or FcRn-binding fragment thereof, wherein the Fc domains of the Fc region or FcRn-binding fragment thereof comprise one or more amino acid substitutions that increase binding to FcRn (e.g., as described herein) relative to a wildtype IgG (e.g., IgG1). In some embodiments, the one or more amino acid substitutions comprise Y, T, E, K, F, and Y at EU positions 252, 254, 256, 433, 434, and 436 respectively. In certain embodiments, the Fc region binds to FcRn with increased affinity and reduced pH dependence relative to a wild-type IgG1 Fc region. Such antibodies and variant Fc also exemplified in U.S. Pat. No. 10,316,073 and Vaccaro C, Zhou J, Ober R J, Ward E S. Engineering the Fc region of immunoglobulin G to modulate in vivo antibody levels. Nat Biotechnology. 2005 October; 23(10):1283-8. doi: 10.1038/nbtl143. In some embodiments, the polypeptide is efgartigimod (ARGX-113). In some embodiments, the polypeptide is efgartigimod (ARGX-113).

Methods for screening and identifying small molecules that bind and/or inhibit FcRn function can be conceived to one skilled in the art. In vitro inhibition of an interaction between FcRn and an IgGFc molecule, or fragment thereof, can be expected to inhibit FcRn function in vivo. Exemplary methods, for example, are disclosed in: “Structure-activity relationships of a peptide inhibitor of the human FcRn:human IgG interaction” Bioorg Med Chem. 2008 Jun. 15; 16(12):6394-405.

Binding of a peptide inhibitor can insufficiently inhibit FcRn function. In some embodiments, the binding affinity of a peptide to FcRn is sufficient to inhibit binding of an IgG molecule. In certain embodiments, the binding affinity of a peptide to FcRn is about 1 nm to about 500 nm. In certain embodiments, the binding affinity of a peptide to FcRn is about 1 nm to about 10 nm, about 1 nm to about 50 nm, about 1 nm to about 100 nm, about 1 nm to about 500 nm, about 1 nm to about 1 nm, about 10 nm to about 50 nm, about 10 nm to about 100 nm, about 10 nm to about 500 nm, about 10 nm to about 1 nm, about 50 nm to about 100 nm, about 50 nm to about 500 nm, about 50 nm to about 1 nm, about 100 nm to about 500 nm, about 100 nm to about 1 nm, or about 500 nm to about 1 nm. In certain embodiments, the binding affinity of a peptide to FcRn is about 1 nm, about 10 nm, about 50 nm, about 100 nm, about 500 nm, or about 1 nm. In certain embodiments, the binding affinity of a peptide to FcRn is at least about 1 nm, about 10 nm, about 50 nm, about 100 nm, or about 500 nm. In certain embodiments, the binding affinity of a peptide to FcRn is at most about 10 nm, about 50 nm, about 100 nm, about 500 nm, or about 1 nm.

The peptide inhibitor can comprise a half-maximal inhibitory concentration (IC50), derived from a competition assay, sufficient to inhibit FcRn function. In certain embodiments, the IC50 value of a peptide inhibitor of FcRn binding to an IgG is about 1 nm to about 1,000,000 nm. In certain embodiments, the IC50 value of a peptide inhibitor of FcRn binding to an IgG is about 1 nm to about 10 nm, about 1 nm to about 100 nm, about 1 nm to about 250 nm, about 1 nm to about 500 nm, about 1 nm to about 750 nm, about 1 nm to about 1,000 nm, about 1 nm to about 5,000 nm, about 1 nm to about 10,000 nm, about 1 nm to about 1,000,000 nm, about 10 nm to about 100 nm, about 10 nm to about 250 nm, about 10 nm to about 500 nm, about 10 nm to about 750 nm, about 10 nm to about 1,000 nm, about 10 nm to about 5,000 nm, about 10 nm to about 10,000 nm, about 10 nm to about 1,000,000 nm, about 100 nm to about 250 nm, about 100 nm to about 500 nm, about 100 nm to about 750 nm, about 100 nm to about 1,000 nm, about 100 nm to about 5,000 nm, about 100 nm to about 10,000 nm, about 100 nm to about 1,000,000 nm, about 250 nm to about 500 nm, about 250 nm to about 750 nm, about 250 nm to about 1,000 nm, about 250 nm to about 5,000 nm, about 250 nm to about 10,000 nm, about 250 nm to about 1,000,000 nm, about 500 nm to about 750 nm, about 500 nm to about 1,000 nm, about 500 nm to about 5,000 nm, about 500 nm to about 10,000 nm, about 500 nm to about 1,000,000 nm, about 750 nm to about 1,000 nm, about 750 nm to about 5,000 nm, about 750 nm to about 10,000 nm, about 750 nm to about 1,000,000 nm, about 1,000 nm to about 5,000 nm, about 1,000 nm to about 10,000 nm, about 1,000 nm to about 1,000,000 nm, about 5,000 nm to about 10,000 nm, about 5,000 nm to about 1,000,000 nm, or about 10,000 nm to about 1,000,000 nm. In certain embodiments, the IC50 value of a peptide inhibitor of FcRn binding to an IgG is about 1 nm, about 10 nm, about 100 nm, about 250 nm, about 500 nm, about 750 nm, about 1,000 nm, about 5,000 nm, about 10,000 nm, or about 1,000,000 nm. In certain embodiments, the IC50 value of a peptide inhibitor of FcRn binding to an IgG is at least about 1 nm, about 10 nm, about 100 nm, about 250 nm, about 500 nm, about 750 nm, about 1,000 nm, about 5,000 nm, or about 10,000 nm. In certain embodiments, the IC50 value of a peptide inhibitor of FcRn binding to an IgG is at most about 10 nm, about 100 nm, about 250 nm, about 500 nm, about 750 nm, about 1,000 nm, about 5,000 nm, about 10,000 nm, or about 1,000,000 nm.

In some embodiments, the peptide inhibitor is selected from TABLE 2.

TABLE 2
FcRn Peptide Inhibitors in FcRn Competition Assay

| | | Standard | Standard | Standard | Standard |
|---|---|---|---|---|---|
| PUBCHEM_SID | PUBCHEM_CID | Type | Relation | Value | Units |
| 103640676 | 44585723 | IC50 | = | 420 | nM |
| 103640836 | 44585845 | IC50 | = | 530 | nM |
| 103640858 | 25090771 | IC50 | = | 570 | nM |
| 103640837 | 44585846 | IC50 | = | 620 | nM |
| 103640832 | 44585841 | IC50 | = | 640 | nM |
| 103640830 | 44585839 | IC50 | = | 660 | nM |
| 103640671 | 44585718 | IC50 | = | 900 | nM |
| 103640675 | 44585722 | IC50 | = | 1000 | nM |
| 103640783 | 44585792 | IC50 | = | 1000 | nM |
| 103640812 | 44585821 | IC50 | = | 1200 | nM |
| 103640762 | 44585771 | IC50 | = | 1300 | nM |
| 103640813 | 44585822 | IC50 | = | 1300 | nM |
| 103640682 | 44585729 | IC50 | = | 1400 | nM |
| 103640672 | 44585719 | IC50 | = | 1600 | nM |
| 103640808 | 44585817 | IC50 | = | 1600 | nM |
| 103640664 | 44585711 | IC50 | = | 1700 | nM |
| 103640767 | 44585776 | IC50 | = | 1700 | nM |
| 103640768 | 44585777 | IC50 | = | 1700 | nM |
| 103640785 | 44585794 | IC50 | = | 1700 | nM |
| 103640818 | 44585827 | IC50 | = | 1700 | nM |
| 103640659 | 44585706 | IC50 | = | 2000 | nM |
| 103640661 | 44585708 | IC50 | = | 2000 | nM |
| 103640662 | 44585709 | IC50 | = | 2000 | nM |
| 103640665 | 44585712 | IC50 | = | 2000 | nM |
| 103640755 | 44585764 | IC50 | = | 2000 | nM |
| 103640674 | 44585721 | IC50 | = | 2100 | nM |
| 103640835 | 44585844 | IC50 | = | 2200 | nM |
| 103640829 | 44585838 | IC50 | = | 2300 | nM |
| 103640688 | 44585735 | IC50 | = | 2400 | nM |
| 103640809 | 44585818 | IC50 | = | 2400 | nM |
| 103640694 | 44585741 | IC50 | = | 2800 | nM |
| 103640689 | 44585736 | IC50 | = | 3100 | nM |
| 103640753 | 44585762 | IC50 | = | 3200 | nM |
| 103640763 | 44585772 | IC50 | = | 3200 | nM |
| 103640692 | 44585739 | IC50 | = | 3600 | nM |
| 103640758 | 44585767 | IC50 | = | 3700 | nM |
| 103640794 | 44585803 | IC50 | = | 3700 | nM |
| 103640793 | 44585802 | IC50 | = | 4000 | nM |
| 103640691 | 44585738 | IC50 | = | 4100 | nM |
| 103640784 | 44585793 | IC50 | = | 4100 | nM |
| 103640760 | 44585769 | IC50 | = | 4300 | nM |
| 103640756 | 44585765 | IC50 | = | 4500 | nM |
| 103640757 | 44585766 | IC50 | = | 4500 | nM |
| 103640819 | 44585828 | IC50 | = | 4600 | nM |
| 103640861 | 44585869 | IC50 | = | 4900 | nM |
| 103640690 | 44585737 | IC50 | = | 5000 | nM |
| 103640817 | 44585826 | IC50 | = | 5500 | nM |
| 103640810 | 44585819 | IC50 | = | 5700 | nM |
| 103640759 | 44585768 | IC50 | = | 5900 | nM |
| 103640811 | 44585820 | IC50 | = | 6200 | nM |
| 103640821 | 44585830 | IC50 | = | 6300 | nM |
| 103640831 | 44585840 | IC50 | = | 7200 | nM |
| 103640859 | 44585867 | IC50 | = | 8000 | nM |
| 103640683 | 44585730 | IC50 | = | 8100 | nM |
| 103640693 | 44585740 | IC50 | = | 9400 | nM |
| 103640841 | 44585850 | IC50 | = | 10000 | nM |
| 103640769 | 44585778 | IC50 | = | 11400 | nM |
| 103640684 | 44585731 | IC50 | = | 12000 | nM |
| 103640815 | 44585824 | IC50 | = | 12000 | nM |
| 103640686 | 44585733 | IC50 | = | 13000 | nM |
| 103640687 | 44585734 | IC50 | = | 13000 | nM |
| 103640775 | 44585784 | IC50 | = | 13000 | nM |
| 103640781 | 44585790 | IC50 | = | 13000 | nM |
| 103640833 | 44585842 | IC50 | = | 13000 | nM |
| 103640828 | 44585837 | IC50 | = | 14000 | nM |
| 103640663 | 44585710 | IC50 | = | 16000 | nM |
| 103640787 | 44585796 | IC50 | = | 16000 | nM |
| 103640840 | 44585849 | IC50 | = | 16000 | nM |
| 174485238 | 73345660 | IC50 | = | 16000 | nM |
| 103640754 | 44585763 | IC50 | = | 17000 | nM |
| 103640660 | 44585707 | IC50 | = | 18000 | nM |
| 103640685 | 44585732 | IC50 | = | 18000 | nM |
| 103640860 | 44585868 | IC50 | = | 19000 | nM |
| 103640655 | 44585702 | IC50 | = | 20000 | nM |
| 103640654 | 44585701 | IC50 | = | 21000 | nM |
| 103640761 | 44585770 | IC50 | = | 21000 | nM |
| 103640766 | 44585775 | IC50 | = | 22000 | nM |
| 103640642 | 44585689 | IC50 | = | 23000 | nM |
| 103640834 | 44585843 | IC50 | = | 23000 | nM |
| 103640857 | 44585866 | IC50 | = | 23000 | nM |
| 103640637 | 44585684 | IC50 | = | 24000 | nM |
| 103640626 | 44585673 | IC50 | = | 26000 | nM |
| 103640790 | 44585799 | IC50 | = | 26000 | nM |
| 103640634 | 44585681 | IC50 | = | 27000 | nM |
| 103640670 | 44585717 | IC50 | = | 27000 | nM |
| 103640823 | 44585832 | IC50 | = | 27000 | nM |
| 103640644 | 44585691 | IC50 | = | 30000 | nM |
| 103640653 | 44585700 | IC50 | = | 30000 | nM |
| 103640852 | 44585861 | IC50 | = | 30000 | nM |
| 103640640 | 44585687 | IC50 | = | 31000 | nM |
| 103640788 | 44585797 | IC50 | = | 31000 | nM |
| 103640847 | 44585856 | IC50 | = | 31000 | nM |
| 103640796 | 44585805 | IC50 | = | 32000 | nM |
| 103640838 | 44585847 | IC50 | = | 32000 | nM |
| 103640628 | 44585675 | IC50 | = | 33000 | nM |
| 103640631 | 44585678 | IC50 | = | 33000 | nM |
| 103640853 | 44585862 | IC50 | = | 33000 | nM |
| 103640639 | 44585686 | IC50 | = | 34000 | nM |
| 103640627 | 44585674 | IC50 | = | 36000 | nM |
| 103640855 | 44585864 | IC50 | = | 37000 | nM |
| 103640856 | 44585865 | IC50 | = | 38000 | nM |
| 103640764 | 44585773 | IC50 | = | 39000 | nM |
| 103640632 | 44585679 | IC50 | = | 40000 | nM |
| 103640816 | 44585825 | IC50 | = | 40000 | nM |
| 103640795 | 44585804 | IC50 | = | 43000 | nM |
| 103640814 | 44585823 | IC50 | = | 45000 | nM |
| 103640678 | 44585725 | IC50 | = | 48000 | nM |
| 103640630 | 44585677 | IC50 | = | 49000 | nM |
| 103640679 | 44585726 | IC50 | = | 57000 | nM |
| 103640826 | 44585835 | IC50 | = | 57000 | nM |
| 103640779 | 44585788 | IC50 | = | 60000 | nM |
| 103640849 | 44585858 | IC50 | = | 60000 | nM |
| 103640629 | 44585676 | IC50 | = | 64000 | nM |
| 103640638 | 44585685 | IC50 | = | 67000 | nM |
| 103640839 | 44585848 | IC50 | = | 68000 | nM |
| 103640680 | 44585727 | IC50 | = | 69000 | nM |
| 103640842 | 44585851 | IC50 | = | 71000 | nM |
| 103640786 | 44585795 | IC50 | = | 80000 | nM |
| 103640844 | 44585853 | IC50 | = | 80000 | nM |
| 103640851 | 44585860 | IC50 | = | 81000 | nM |
| 103640822 | 44585831 | IC50 | = | 85000 | nM |
| 103640843 | 44585852 | IC50 | = | 86000 | nM |
| 103640641 | 44585688 | IC50 | = | 87000 | nM |
| 103640778 | 44585787 | IC50 | = | 90000 | nM |
| 103640782 | 44585791 | IC50 | = | 90000 | nM |
| 103640805 | 44585814 | IC50 | = | 92000 | nM |
| 103640643 | 44585690 | IC50 | = | 95000 | nM |
| 103640652 | 44585699 | IC50 | = | 96000 | nM |
| 103640776 | 44585785 | IC50 | = | 100000 | nM |
| 103640824 | 44585833 | IC50 | > | 100000 | nM |
| 103640845 | 44585854 | IC50 | = | 100000 | nM |
| 103640650 | 44585697 | IC50 | = | 107000 | nM |
| 103640635 | 44585682 | IC50 | = | 110000 | nM |
| 103640801 | 44585810 | IC50 | = | 110000 | nM |
| 103640777 | 44585786 | IC50 | = | 120000 | nM |
| 103640802 | 44585811 | IC50 | = | 120000 | nM |
| 103640846 | 44585855 | IC50 | = | 120000 | nM |
| 103640848 | 44585857 | IC50 | = | 120000 | nM |
| 103640806 | 44585815 | IC50 | = | 122000 | nM |
| 103640645 | 44585692 | IC50 | > | 125000 | nM |
| 103640646 | 44585693 | IC50 | > | 125000 | nM |
| 103640647 | 44585694 | IC50 | > | 125000 | nM |
| 103640648 | 44585695 | IC50 | > | 125000 | nM |
| 103640649 | 44585696 | IC50 | > | 125000 | nM |
| 103640651 | 44585698 | IC50 | > | 125000 | nM |
| 103640656 | 44585703 | IC50 | > | 125000 | nM |
| 103640657 | 44585704 | IC50 | = | 125000 | nM |
| 103640667 | 44585714 | IC50 | > | 125000 | nM |
| 103640669 | 44585716 | IC50 | > | 125000 | nM |
| 103640673 | 44585720 | IC50 | > | 125000 | nM |
| 103640677 | 44585724 | IC50 | > | 125000 | nM |
| 103640681 | 44585728 | IC50 | > | 125000 | nM |
| 103640770 | 44585779 | IC50 | > | 125000 | nM |
| 103640771 | 44585780 | IC50 | > | 125000 | nM |
| 103640772 | 44585781 | IC50 | > | 125000 | nM |
| 103640773 | 44585782 | IC50 | > | 125000 | nM |
| 103640774 | 44585783 | IC50 | > | 125000 | nM |
| 103640780 | 44585789 | IC50 | > | 125000 | nM |
| 103640789 | 44585798 | IC50 | > | 125000 | nM |
| 103640791 | 44585800 | IC50 | > | 125000 | nM |
| 103640792 | 44585801 | IC50 | > | 125000 | nM |
| 103640797 | 44585806 | IC50 | > | 125000 | nM |
| 103640798 | 44585807 | IC50 | > | 125000 | nM |
| 103640800 | 44585809 | IC50 | > | 125000 | nM |
| 103640803 | 44585812 | IC50 | > | 125000 | nM |
| 103640804 | 44585813 | IC50 | > | 125000 | nM |
| 103640807 | 44585816 | IC50 | > | 125000 | nM |
| 103640825 | 44585834 | IC50 | > | 125000 | nM |
| 103640827 | 44585836 | IC50 | > | 125000 | nM |
| 103640850 | 44585859 | IC50 | > | 125000 | nM |
| 103640854 | 44585863 | IC50 | > | 125000 | nM |
| 103640820 | 44585829 | IC50 | = | 150000 | nM |
| 103640666 | 44585713 | IC50 | = | 169000 | nM |
| 103640633 | 44585680 | IC50 | = | 239000 | nM |
| 103640636 | 44585683 | IC50 | > | 250000 | nM |
| 103640668 | 44585715 | IC50 | > | 250000 | nM |
| 103640765 | 44585774 | IC50 | > | 250000 | nM |
| 103640658 | 44585705 | IC50 | > | 500000 | nM |

A protein or polypeptide can also be used as an inhibitor of FcRn function. In some embodiments, a protein inhibiting FcRn function comprises a protein that inhibits the transfer of maternal antibodies across a placental barrier. In certain embodiments, inhibiting FcRn function comprises a protein that inhibits transfer of maternal antibodies to a region wherein the maternal antibodies can contact an embryo or fetus. In certain embodiments, a protein inhibiting FcRn function comprises a protein that inhibits FcRn-mediated IgG transfer across or through endosomal vesicles in the syncytiotrophoblast of the placenta. In some embodiments, a protein inhibiting FcRn function comprises a protein that inhibits or blocks the interaction between FcRn and an antibody molecule. In some embodiments, a protein inhibiting FcRn function comprises a protein that inhibits, blocks, and/or reduces FcRn-mediated recycling or rescue of an antibody molecule. In some embodiments, a protein inhibiting FcRn function comprises a protein that inhibits accessory molecules involved in FcRn function.

In some embodiments, the binding affinity of a protein to FcRn is sufficient to inhibit binding of an IgG molecule. In certain embodiments, the binding affinity of a protein to FcRn is about 1 nm to about 500 nm. In certain embodiments, the binding affinity of a protein to FcRn is about 1 nm to about 10 nm, about 1 nm to about 50 nm, about 1 nm to about 100 nm, about 1 nm to about 500 nm, about 1 nm to about 1 nm, about 10 nm to about 50 nm, about 10 nm to about 100 nm, about 10 nm to about 500 nm, about 10 nm to about 1 nm, about 50 nm to about 100 nm, about 50 nm to about 500 nm, about 50 nm to about 1 nm, about 100 nm to about 500 nm, about 100 nm to about 1 nm, or about 500 nm to about 1 nm. In certain embodiments, the binding affinity of a protein to FcRn is about 1 nm, about 10 nm, about 50 nm, about 100 nm, about 500 nm, or about 1 nm. In certain embodiments, the binding affinity of a protein to FcRn is at least about 1 nm, about 10 nm, about 50 nm, about 100 nm, or about 500 nm. In certain embodiments, the binding affinity of a protein to FcRn is at most about 10 nm, about 50 nm, about 100 nm, about 500 nm, or about 1 nm.

The protein inhibitor can comprise half-maximal inhibitory concentration (IC50), derived from a competition assay, sufficient to inhibit FcRn function. In certain embodiments, the IC50 value of a protein inhibitor of FcRn binding to an IgG is about 1 nm to about 1,000,000 nm. In certain embodiments, the IC50 value of a protein inhibitor of FcRn binding to an IgG is about 1 nm to about 10 nm, about 1 nm to about 100 nm, about 1 nm to about 250 nm, about 1 nm to about 500 nm, about 1 nm to about 750 nm, about 1 nm to about 1,000 nm, about 1 nm to about 5,000 nm, about 1 nm to about 10,000 nm, about 1 nm to about 1,000,000 nm, about 10 nm to about 100 nm, about 10 nm to about 250 nm, about 10 nm to about 500 nm, about 10 nm to about 750 nm, about 10 nm to about 1,000 nm, about 10 nm to about 5,000 nm, about 10 nm to about 10,000 nm, about 10 nm to about 1,000,000 nm, about 100 nm to about 250 nm, about 100 nm to about 500 nm, about 100 nm to about 750 nm, about 100 nm to about 1,000 nm, about 100 nm to about 5,000 nm, about 100 nm to about 10,000 nm, about 100 nm to about 1,000,000 nm, about 250 nm to about 500 nm, about 250 nm to about 750 nm, about 250 nm to about 1,000 nm, about 250 nm to about 5,000 nm, about 250 nm to about 10,000 nm, about 250 nm to about 1,000,000 nm, about 500 nm to about 750 nm, about 500 nm to about 1,000 nm, about 500 nm to about 5,000 nm, about 500 nm to about 10,000 nm, about 500 nm to about 1,000,000 nm, about 750 nm to about 1,000 nm, about 750 nm to about 5,000 nm, about 750 nm to about 10,000 nm, about 750 nm to about 1,000,000 nm, about 1,000 nm to about 5,000 nm, about 1,000 nm to about 10,000 nm, about 1,000 nm to about 1,000,000 nm, about 5,000 nm to about 10,000 nm, about 5,000 nm to about 1,000,000 nm, or about 10,000 nm to about 1,000,000 nm. In certain embodiments, the IC50 value of a protein inhibitor of FcRn binding to an IgG is about 1 nm, about 10 nm, about 100 nm, about 250 nm, about 500 nm, about 750 nm, about 1,000 nm, about 5,000 nm, about 10,000 nm, or about 1,000,000 nm. In certain embodiments, the IC50 value of a protein inhibitor of FcRn binding to an IgG is at least about 1 nm, about 10 nm, about 100 nm, about 250 nm, about 500 nm, about 750 nm, about 1,000 nm, about 5,000 nm, or about 10,000 nm. In certain embodiments, the IC50 value of a protein inhibitor of FcRn binding to an IgG is at most about 10 nm, about 100 nm, about 250 nm, about 500 nm, about 750 nm, about 1,000 nm, about 5,000 nm, about 10,000 nm, or about 1,000,000 nm.

An Fc region of an IgG molecule or mutant or variant thereof can be used to inhibit FcRn function. In some embodiments, the protein is the Fc region of an IgG molecule (Fc molecule). In certain embodiments, the Fc molecule exhibits increased binding to an FcRn molecule as compared to a wild-type Fc region of an IgG. In certain embodiments, the Fc molecule comprises at least one mutation, relative to wild-type (as disclosed in: Selection of IgG Variants with Increased FcRn Binding Using Random and Directed Mutagenesis: Impact on Effector Functions” Front Immunol. 2015; 6: 39. Published online 2015 Feb. 4; or “IgG Fc engineering to modulate antibody effector functions” Protein Cell. 2018 January; 9(1):63-73. doi: 10.1007/s13238-017-0473-8. Epub 2017 Oct. 6). In some embodiments, the inhibitor of FcRn function is a variant Fc molecule or FcRn-binding fragment thereof, wherein the Fc region or fragment comprises the amino acids Y, T, E, K, F, and Y at EU positions 252, 254, 256, 433, 434, and 436 respectively. In certain embodiments, and the variant Fc molecule is not a full-length antibody (e.g., an Abdeg). In certain embodiments, variant Fc molecule does not comprise an antibody variable region or a CH1 domain. In certain embodiments, variant Fc molecule does not comprise a free cysteine residue. In certain embodiments, the variant Fc molecule is derived from an IgG Fc region (e.g., a human IgG Fc region having one or more mutations). In certain embodiments, the variant Fc molecule is derived from an IgG1 Fc region (e.g., a human IgG1 Fc region having one or more mutations). In certain embodiments, the Fc region is a chimeric Fc region.

In certain embodiments, the variant Fc molecule comprises amino acid sequence set forth in SEQ ID NO:1. In certain embodiments, the variant Fc molecule comprises the amino acid sequence set forth in SEQ ID NO: 1, 2, or 3. In certain embodiments, the variant Fc molecule comprises the amino acid sequence set forth in SEQ ID NO: 2. In certain embodiments, variant Fc molecule that has altered increased affinity (i.e., stronger binding) for an Fc receptor relative to the affinity of a wild-type IgG1 Fc region

In certain embodiments, the FcRn antagonist comprises a variant Fc region that does not comprise an N-linked glycan at EU position 297. In certain embodiments, the FcRn antagonist comprises a variant Fc region that comprises an afucosylated N-linked glycan at EU position 297. In certain embodiments, the FcRn antagonist comprises a variant Fc region that comprises an N-linked glycan having a bisecting GlcNac at EU position 297. In certain embodiments, the FcRn antagonist comprises a variant Fc region linked to a half-life extender. In certain embodiments, the half-life extender is polyethylene glycol or human serum albumin.

In some embodiments, a reduction of auto-reactive antibodies can be achieved by reducing the number of auto-reactive antibodies capable of binding a fetal antigen. In such embodiments, a polypeptide comprising an epitope that is bound by the auto-reactive antibody is administered and thereby reduces the number of auto-reactive antibodies capable of binding a fetal antigen. In certain embodiments, the polypeptide is selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, and ezrin (EZR).

In some embodiments, a reduction of auto-reactive antibodies can be achieved by reducing the number of auto-reactive antibodies capable of binding a fetal antigen. In such embodiments, a polypeptide comprising an epitope that is bound by the auto-reactive antibody is administered and thereby reduces the number of auto-reactive antibodies capable of binding a fetal antigen. In certain embodiments, the polypeptide is selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, and ezrin (EZR).

The polypeptide described herein can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. Therapeutically effective amounts include amounts are those sufficient to ameliorate one or more symptoms associated with the disease or affliction to be treated. APTAMERS (aka synthetic antibodies) or similar single stranded DNA or RNA oligonucleotides, in a form that can be administered to as a human therapeutic, that might bind to and inhibit FcRn function.

### Nucleic Acid-Based Inhibitors

Methods as disclosed herein for inhibiting of FcRn function within the pregnant individual, individual trying to become pregnant, or considering pregnancy are characterized by the administration of an FcRn inhibitor, wherein the FcRn inhibitor comprises a nucleic acid molecule. Nucleic acid molecules can inhibit FcR perturbing the process of transcription, or post-transcriptional events, including messenger ribonucleic acid (mRNA) processing and translation. Thus, nucleic acid molecules can be a deoxyribonucleic acid (DNA) molecule or a ribonucleic acid (RNA molecule). In some embodiments, the nucleic acid molecule reduces FcRn gene expression in a cell expressing FcRn. In certain embodiments, the nucleic acid molecule reduces FcRn mRNA expression in a cell expressing FcRn. In certain embodiments, the nucleic acid molecule reduces FcRn protein expression in a cell expressing FcRn. In certain embodiments, targeting mRNA encoding an FcRn molecule can be accomplished by use of antisense DNA, antisense RNA, short hairpin RNA, interfering RNA, or RNA-decoy molecules.

Inhibition of FcRn can also be achieved by providing a cell with a nucleic acid molecule encoding an FcRn inhibitor. In some embodiments the nucleic acid molecule encodes a protein molecule. In some embodiments, the nucleic acid molecule encodes an antibody or target binding fragment thereof. In some embodiments, a viral vector comprises the nucleic acid molecule is delivered. I some embodiments, the comprises an expression vector that comprises the nucleic acid molecule.

Methods for inhibiting FcRn function can also include targeting the FcRn gene to reduce expression of FcRn. For example, DNA endonucleases that may be used include, e.g., a Cas9 endonuclease, a zinc finger nuclease, a transcription activator-like effector nuclease (TALEN), a homing endonuclease, a dCas9-FokI nuclease or a MegaTal nuclease. DNA endonucleases may be introduced into a cell expressing FcRn by a variety of means, including by the introduction and/or expression one or more polynucleotides encoding the DNA endonuclease, as known in the art and as described and illustrated further herein. In certain embodiments, DNA endonucleases and/or other components of the genome editing systems, such as guide RNAs in the embodiment of Cas9 genome editing, are encoded by RNAs introduced into the cells expressing FcRn. In some embodiments, the DNA endonuclease is a Cas9 endonuclease and the method comprises introducing into the cell one or more polynucleotides encoding Cas9 and at least one guide RNA, that targets the FcRn gene. In other embodiments, the DNA endonuclease is a zinc finger nuclease (ZFN) and the method comprises introducing into the cell expressing FcRn one or more polynucleotides encoding a ZFNs that targets the FcRn gene. Alternatively, TALENs or other endonucleases may be used.

### Administration

Methods, as disclosed herein, for inhibiting FcRn function and/or reducing auto-reactive antibodies capable of contacting an embryo and/or fetus of a pregnant individual, an individual trying to become pregnant, an individual considering pregnancy, or an individual serving as a surrogate for the pregnancy, are characterized by the administering an inhibitor of FcRn function to the pregnant individual, individual trying to become pregnant, or considering pregnancy. Administering, as disclosed, can also be used for reducing auto-reactive antibodies by preventing an interaction between an IgG molecule and FcRn in a pregnant individual, an individual trying to become pregnant, an individual considering pregnancy, or an individual serving as a surrogate for the pregnancy. In some embodiments, administering an FcRn inhibitor prevents or inhibits the transfer of maternal antibodies across a placental barrier. In certain embodiments, administering an FcRn inhibitor prevents or inhibits the transfer of maternal antibodies to a region wherein the antibodies can contact an embryo or fetus. In certain embodiments, administering an FcRn inhibitor prevents or inhibits FcRn-mediated IgG transfer across or through endosomal vesicles in the syncytiotrophoblast of the placenta. In some embodiments administering an FcRn inhibitor prevents or inhibits or blocks the interaction between FcRn and an antibody molecule. In some embodiments, administering an FcRn inhibitor prevents or inhibits, blocks, and/or reduces FcRn-mediated recycling or rescue of an antibody molecule. In some embodiments, administering an FcRn inhibitor prevents or inhibits accessory molecules involved in FcRn function.

In certain embodiments, the anti-FcRn antibodies and FcRn inhibiting molecules can be administered to a subject in need thereof by any route suitable for the administration of antibody-containing pharmaceutical compositions, such as, for example, subcutaneous, intraperitoneal, intravenous, or intramuscular. In certain embodiments, the anti-FcRn antibodies and FcRn inhibiting molecules are administered intravenously. In certain embodiments, the anti-FcRn antibodies and FcRn inhibiting molecules are administered subcutaneously.

FcRn inhibitors can be administered through a variety of designed schedules for inhibiting FcRn function before or during pregnancy. The inhibitor of FcRn function can be administered to an individual anytime during pregnancy. In some embodiments, inhibitor of FcRn function is administered after the fetal brain has begun to develop, e.g., after about the 12 week of gestation. In some embodiments, inhibitor of FcRn function is administered before the fetal brain has begun to develop. In some embodiments, administering is performed at least once over the course of a pregnancy. In some embodiments, administering is performed at once during each trimester of a pregnancy. In certain embodiments, administering is performed at once during the first trimester. In certain embodiments, administering is performed at once during the second trimester. In certain embodiments, administering is performed at once during the third trimester. In some embodiments, administration of the FcRn inhibitor is performed daily during pregnancy. In some embodiments, administration of the FcRn inhibitor is performed daily prior to pregnancy. In some embodiments, administration of the FcRn inhibitor is performed weekly during pregnancy. In some embodiments, administration of the FcRn inhibitor is performed weekly prior to pregnancy. In some embodiments, administration of the FcRn inhibitor is performed monthly during pregnancy. In some embodiments, administration of the FcRn inhibitor is performed monthly prior to pregnancy.

Administration strategies can be applied prior to an individual becoming pregnant. In some embodiments, administering is performed at least once before an individual becomes pregnant. In certain embodiments, administering is performed about 1 time before becoming pregnant to about 10 times before becoming pregnant. In certain embodiments, administering is performed about 1 time before becoming pregnant to about 2 times before becoming pregnant, about 1 time before becoming pregnant to about 3 times before becoming pregnant, about 1 time before becoming pregnant to about 4 times before becoming pregnant, about 1 time before becoming pregnant to about 5 times before becoming pregnant, about 1 time before becoming pregnant to about 10 times before becoming pregnant, about 2 times before becoming pregnant to about 3 times before becoming pregnant, about 2 times before becoming pregnant to about 4 times before becoming pregnant, about 2 times before becoming pregnant to about 5 times before becoming pregnant, about 2 times before becoming pregnant to about 10 times before becoming pregnant, about 3 times before becoming pregnant to about 4 times before becoming pregnant, about 3 times before becoming pregnant to about 5 times before becoming pregnant, about 3 times before becoming pregnant to about 10 times before becoming pregnant, about 4 times before becoming pregnant to about 5 times before becoming pregnant, about 4 times before becoming pregnant to about 10 times before becoming pregnant, or about 5 times before becoming pregnant to about 10 times before becoming pregnant. In certain embodiments, administering is performed about 1 time before becoming pregnant, about 2 times before becoming pregnant, about 3 times before becoming pregnant, about 4 times before becoming pregnant, about 5 times before becoming pregnant, or about 10 times before becoming pregnant. In certain embodiments, administering is performed at least about 1 time before becoming pregnant, about 2 times before becoming pregnant, about 3 times before becoming pregnant, about 4 times before becoming pregnant, or about 5 times before becoming pregnant. In certain embodiments, administering is performed at most about 2 times before becoming pregnant, about 3 times before becoming pregnant, about 4 times before becoming pregnant, about 5 times before becoming pregnant, or about 10 times before becoming pregnant.

FcRn inhibitors can be administered through a variety of designed schedules for inhibiting FcRn function before or during pregnancy. In some embodiments, the FcRn inhibitor is administered prior to an individual becoming pregnant. In some embodiments, the FcRn inhibitor is administered about 0 days prior to conception to about 100 days prior to conception. In some embodiments, the FcRn inhibitor is administered about 0 days prior to conception to about 10 days prior to conception, about 0 days prior to conception to about 20 days prior to conception, about 0 days prior to conception to about 30 days prior to conception, about 0 days prior to conception to about 40 days prior to conception, about 0 days prior to conception to about 60 days prior to conception, about 0 days prior to conception to about 100 days prior to conception, about 10 days prior to conception to about 20 days prior to conception, about 10 days prior to conception to about 30 days prior to conception, about 10 days prior to conception to about 40 days prior to conception, about 10 days prior to conception to about 60 days prior to conception, about 10 days prior to conception to about 100 days prior to conception, about 20 days prior to conception to about 30 days prior to conception, about 20 days prior to conception to about 40 days prior to conception, about 20 days prior to conception to about 60 days prior to conception, about 20 days prior to conception to about 100 days prior to conception, about 30 days prior to conception to about 40 days prior to conception, about 30 days prior to conception to about 60 days prior to conception, about 30 days prior to conception to about 100 days prior to conception, about 40 days prior to conception to about 60 days prior to conception, about 40 days prior to conception to about 100 days prior to conception, or about 60 days prior to conception to about 100 days prior to conception. In some embodiments, the FcRn inhibitor is administered about 0 days prior to conception, about 10 days prior to conception, about 20 days prior to conception, about 30 days prior to conception, about 40 days prior to conception, about 60 days prior to conception, or about 100 days prior to conception. In some embodiments, the FcRn inhibitor is administered at least about 0 days prior to conception, about 10 days prior to conception, about 20 days prior to conception, about 30 days prior to conception, about 40 days prior to conception, or about 60 days prior to conception. In some embodiments, the FcRn inhibitor is administered at most about 10 days prior to conception, about 20 days prior to conception, about 30 days prior to conception, about 40 days prior to conception, about 60 days prior to conception, or about 100 days prior to conception. In some embodiments, the FcRn inhibitor is administered on the date of conception.

The FcRn inhibitor can be administered during pregnancy or after conception. In some embodiments the FcRn inhibitor is administered during pregnancy or after conception. In some embodiments, gestational age of a fetus of the pregnant individual is less than about 30, 60, 90, or 100 days. In some embodiments, the gestational age of a fetus of the pregnant individual is greater than about 100, 150, or 200 days. In some embodiments, the gestational age of a fetus of the pregnant individual is about 0 days to about 200 days. In some embodiments, the gestational age of a fetus of the pregnant individual is about 0 days to about 10 days, about 0 days to about 20 days, about 0 days to about 30 days, about 0 days to about 40 days, about 0 days to about 60 days, about 0 days to about 100 days, about 0 days to about 150 days, about 0 days to about 200 days, about 10 days to about 20 days, about 10 days to about 30 days, about 10 days to about 40 days, about 10 days to about 60 days, about 10 days to about 100 days, about 10 days to about 150 days, about 10 days to about 200 days, about 20 days to about 30 days, about 20 days to about 40 days, about 20 days to about 60 days, about 20 days to about 100 days, about 20 days to about 150 days, about 20 days to about 200 days, about 30 days to about 40 days, about 30 days to about 60 days, about 30 days to about 100 days, about 30 days to about 150 days, about 30 days to about 200 days, about 40 days to about 60 days, about 40 days to about 100 days, about 40 days to about 150 days, about 40 days to about 200 days, about 60 days to about 100 days, about 60 days to about 150 days, about 60 days to about 200 days, about 100 days to about 150 days, about 100 days to about 200 days, or about 150 days to about 200 days. In some embodiments, the gestational age of a fetus of the pregnant individual is about 0 days, about 10 days, about 20 days, about 30 days, about 40 days, about 60 days, about 100 days, about 150 days, or about 200 days. In some embodiments, the gestational age of a fetus of the pregnant individual is at least about 0 days, about 10 days, about 20 days, about 30 days, about 40 days, about 60 days, about 100 days, or about 150 days. In some embodiments, the gestational age of a fetus of the pregnant individual is at most about 10 days, about 20 days, about 30 days, about 40 days, about 60 days, about 100 days, about 150 days, or about 200 days.

In certain embodiments the anti-FcRn antibodies or molecules of the current disclosure are included in a pharmaceutical composition comprising one or more pharmaceutically acceptable excipients, carriers, and diluents. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies or molecules of the current disclosure are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

### Pregnant Individual

The term “pregnant individual” can refer to an individual who is pregnant or having an embryo or fetus or child or young developing within the individual. The diagnosis of pregnancy or gestation can be readily determined by the individual or a physician using tests and/or procedures known within the art. A “surrogate” or “pregnancy surrogate” refers to a female individual who has had a fertilized embryo implanted for the purposes of initiating a pregnancy. Generally, the genetic make-up of the embryo will be the result of in vitro fertilization of two individuals that are not the surrogate.

The term “trying to become pregnant” can refer to an individual attempting to become or actively pursuing pregnancy. In some embodiments, trying to become pregnant can include sexual intercourse without the use of birth control or contraception. In some embodiments, trying to become pregnant can include consultation with a physician or family planning counselor. In some embodiments, trying to become pregnant can include the intake of prenatal vitamins or supplements, by the individual, that are associated with aiding, promoting, or bolstering the state of being pregnant. In some embodiments, an individual trying to become pregnant is undergoing or plans to under an in vitro fertilization procedure.

The term “considering becoming pregnant” or “considering pregnancy” can refer to an individual contemplating pregnancy. In some embodiments, considering pregnancy can include sexual intercourse without the use of birth control or contraception. In some embodiments, considering pregnancy can include consultation with a physician or family planning counselor. In some embodiments, trying to become pregnant can include the individual purchasing or intaking prenatal vitamins or that are associated with to aiding, promoting, or bolstering the state of being pregnant.

Past pregnancies or offspring of a pregnant individual, an individual trying to become pregnant, an individual considering pregnancy or serving as a surrogate to carry the pregnancy, can affect the likelihood and/or risk that auto-reactive antibodies are present within the individual. In some embodiments, the pregnant individual, an individual trying to become pregnant, an individual considering pregnancy, or an individual serving as a surrogate for the pregnancy, has birthed at least one child diagnosed with ASD.

The age of a pregnant individual, an individual trying to become pregnant, an individual considering pregnancy, or an individual serving as a surrogate for the pregnancy, can affect the likelihood and/or risk that auto-reactive antibodies are present within the individual. In various embodiments, the method can be dependent on the age of the individual age of a pregnant individual, an individual trying to become pregnant, an individual considering pregnancy, or an individual serving as a surrogate for the pregnancy. In some embodiments, a pregnant individual, an individual trying to become pregnant, an individual considering pregnancy, or an individual serving as a surrogate for the pregnancy, is about 30 years to about 45 years. In some embodiments, a pregnant individual, an individual trying to become pregnant, an individual considering pregnancy, or an individual serving as a surrogate for the pregnancy, is about 30 years to about 35 years, about 30 years to about 40 years, about 30 years to about 45 years, about 35 years to about 40 years, about 35 years to about 45 years, or about 40 years to about 45 years. In some embodiments, a pregnant individual, an individual trying to become pregnant, an individual considering pregnancy, or an individual serving as a surrogate for the pregnancy, is about 30 years, about 35 years, about 40 years, or about 45 years. In some embodiments, a pregnant individual, an individual trying to become pregnant, an individual considering pregnancy, or an individual serving as a surrogate for the pregnancy, is at least about 30 years, about 35 years, or about 40 years. In some embodiments, a pregnant individual, an individual trying to become pregnant, an individual considering pregnancy, or an individual serving as a surrogate for the pregnancy, is at most about 35 years, about 40 years, or about 45 years.

The age of the individual wherein the sperm is derived can affect ASD risk. In some embodiments, the of age of an individual from which the sperm was provided or to be provided from. In certain embodiments, the age of a person that provides or provided a sperm to fertilize an egg of the pregnant individual, individual trying to become pregnant, or individual considering pregnancy, or an individual serving as a surrogate for the pregnancy, is about 30 years to about 50 years. In certain embodiments, the age of a person that provides or provided a sperm to fertilize an egg of the pregnant individual, individual trying to become pregnant, or individual considering pregnancy, or an individual serving as a surrogate for the pregnancy, is about 30 years to about 35 years, about 30 years to about 40 years, about 30 years to about 45 years, about 30 years to about 50 years, about 35 years to about 40 years, about 35 years to about 45 years, about 35 years to about 50 years, about 40 years to about 45 years, about 40 years to about 50 years, or about 45 years to about 50 years. In certain embodiments, the age of a person that provides or provided a sperm to fertilize an egg of the pregnant individual, individual trying to become pregnant, or individual considering pregnancy, or an individual serving as a surrogate for the pregnancy, is about 30 years, about 35 years, about 40 years, about 45 years, or about 50 years. In certain embodiments, the age of a person that provides or provided a sperm to fertilize an egg of the pregnant individual, individual trying to become pregnant, or individual considering pregnancy, or an individual serving as a surrogate for the pregnancy, is at least about 30 years, about 35 years, about 40 years, or about 45 years. In certain embodiments, the age of a person that provides or provided a sperm to fertilize an egg of the pregnant individual, individual trying to become pregnant, or individual considering pregnancy, or an individual serving as a surrogate for the pregnancy, is at most about 35 years, about 40 years, about 45 years, or about 50 years.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some embodiments, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptom s associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

Thus, disclosed herein is a method of reducing auto-reactive antibodies (e.g., an amount present in blood or sera) in a pregnant individual or an individual trying to become or an individual considering pregnancy, or an individual serving as a surrogate for the pregnancy, comprising administering, to the pregnant individual or individual trying to become pregnant or individual considering pregnancy, or an individual serving as a surrogate for the pregnancy, an inhibitor of neonatal Fc receptor (FcRn) function.

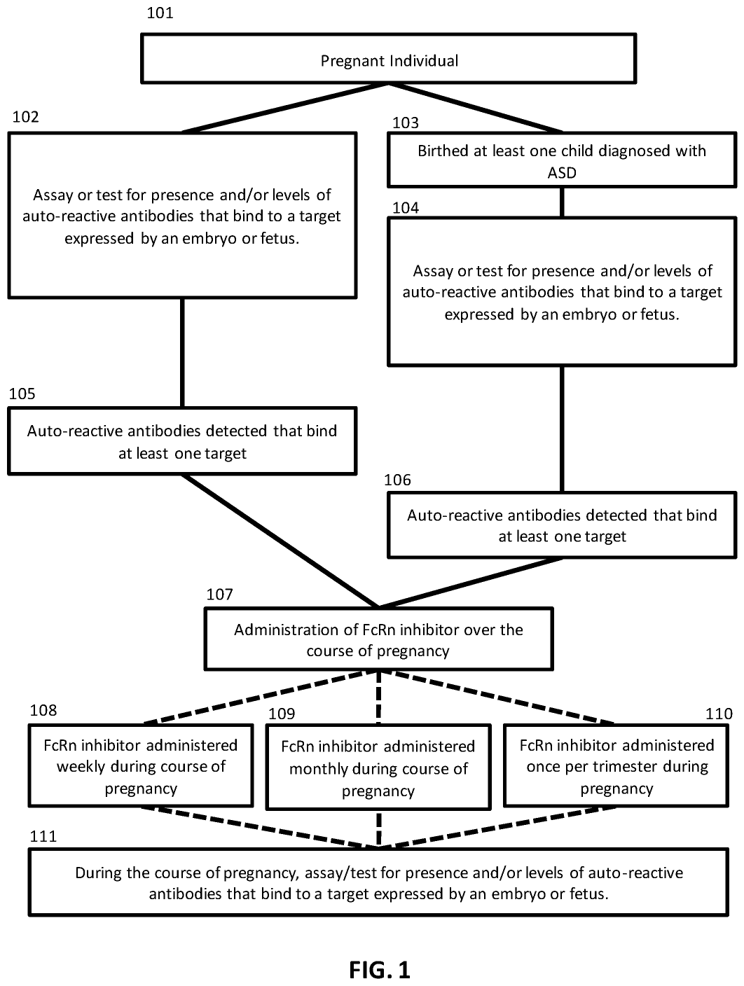

FIG. 1 illustrates a schematic for clinical diagnosis and administration of an FcRn inhibitor to a pregnant individual. The pregnant individual or a biological sample from the pregnant individual can be assayed or tested for the presence of auto-reactive antibodies (see, e.g., 102 and/or 104). In 102 and/or 104, A sample from a pregnant individual can be assayed for the presence of maternal autoantibodies that bind to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In some cases, it is known that the pregnant individual has birthed at least one child diagnosed with ASD (103). The presence of at least one maternal autoantibody in the assay of 102 and/or 104 would result in the clinical decision to administer the FcRn inhibitor (107). Schedules for the administration of the FcRn inhibitor can be designed wherein the FcRn is administered weekly during pregnancy, monthly during pregnancy, or once per trimester (see 108, 109, and 110, respectively; dotted line indicates the option of more than one schedule available for administration). During the course of pregnancy and/or following the administration of the FcRn inhibitor, the pregnant individual can be tested and/or monitored for presence and/or levels of auto-reactive antibodies that bind to a target expressed by an embryo or fetus (see 111).

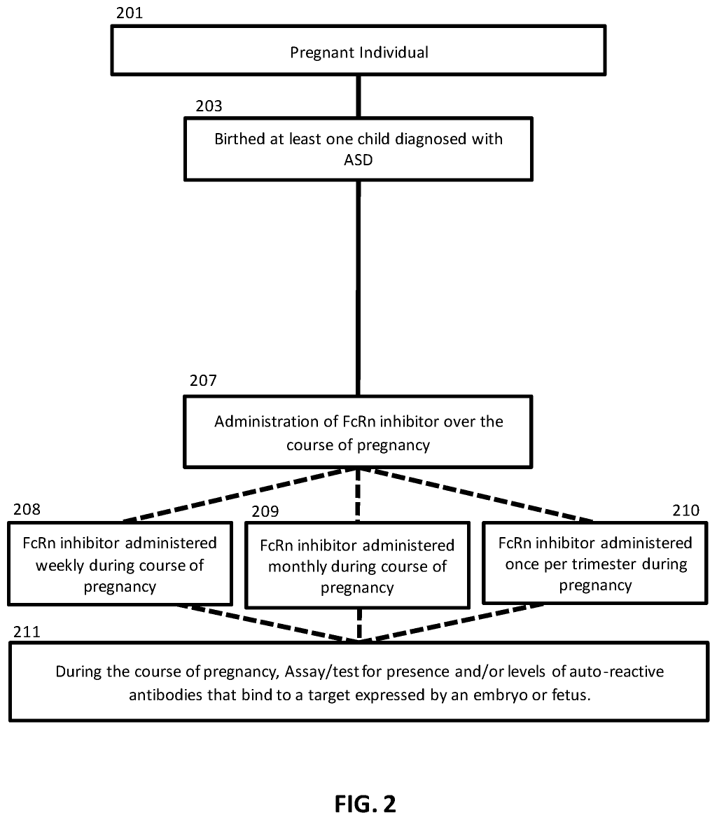

FIG. 2 illustrates a schematic for clinical diagnosis and administration of an FcRn inhibitor to a pregnant individual. In cases where it is known that the pregnant individual has birthed at least one child diagnosed with ASD (203), a clinical decision is made to administer the FcRn inhibitor (207). Schedules for the administration of the FcRn inhibitor can be designed wherein the FcRn is administered weekly during pregnancy, monthly during pregnancy, or once per trimester (see 208, 209, and 210, respectively; dotted line indicates the option of more than one schedule available for administration). During the course of pregnancy and/or following the administration of the FcRn inhibitor, the pregnant individual can be tested and/or monitored for presence and/or levels of auto-reactive antibodies that bind to a target expressed by an embryo or fetus (see 211).

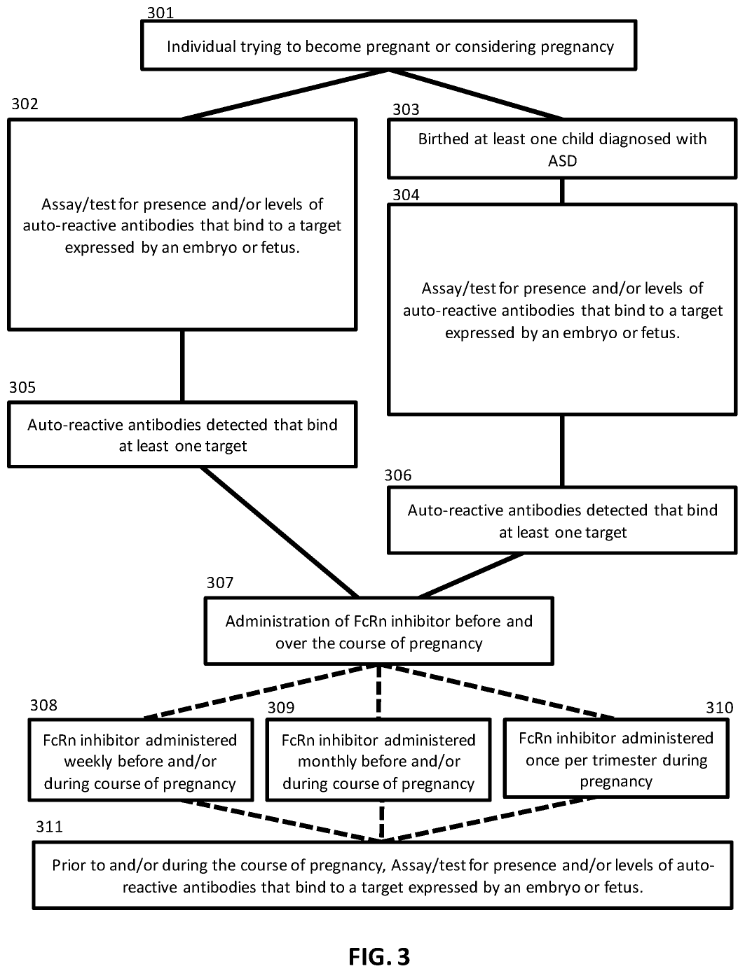

FIG. 3 illustrates a schematic for clinical diagnosis and administration of an FcRn inhibitor to an individual trying to become pregnant or considering pregnancy. The individual trying to become pregnant or considering pregnancy or a biological sample from the individual can be assayed or tested for the presence of auto-reactive antibodies (see, e.g., 302 and/or 304). In 302 and/or 304, A sample from a pregnant individual can be assayed for the presence of maternal autoantibodies that bind to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In some cases, it is known that the individual trying to become pregnant or considering pregnancy has birthed at least one child diagnosed with ASD (303). The presence of at least one maternal autoantibody in the assay of 302 and/or 304 would result in the clinical decision to administer the FcRn inhibitor before and/or during pregnancy (307). Schedules for the administration of the FcRn inhibitor can be designed wherein the FcRn is administered weekly before pregnancy, monthly before pregnancy, weekly during pregnancy, monthly during pregnancy, and/or once per trimester (see 308, 309, and 310, respectively; dotted line indicates the option of more than one schedule available for administration). Prior to and/or during the course of pregnancy and/or following the administration of the FcRn inhibitor, the individual trying to become pregnant or considering pregnancy can be tested and/or monitored for presence and/or levels of auto-reactive antibodies that bind to a target expressed by an embryo or fetus (see 311).

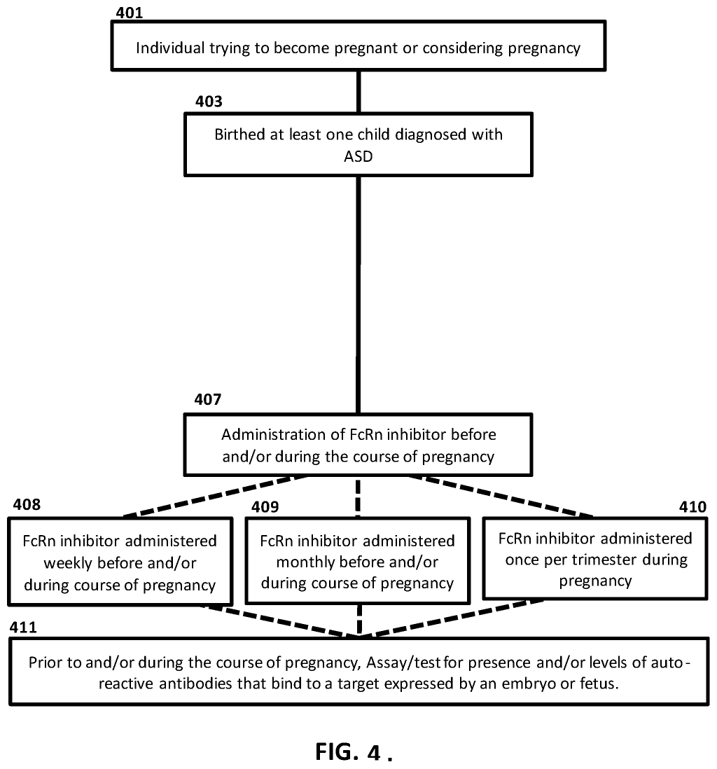

FIG. 4 illustrates a schematic for clinical diagnosis and administration of an FcRn inhibitor to an individual trying to become pregnant or considering pregnancy. In cases where it is known that the individual trying to become pregnant or considering pregnancy has birthed at least one child diagnosed with ASD (403), a clinical decision is made to administer the FcRn inhibitor (407). Schedules for the administration of the FcRn inhibitor can be designed wherein the FcRn is administered weekly before pregnancy, monthly before pregnancy, weekly during pregnancy, monthly during pregnancy, and/or once per trimester (see 408, 409, and 410, respectively; dotted line indicates the option of more than one schedule available for administration). Prior to and/or during the course of pregnancy and/or following the administration of the FcRn inhibitor, the individual trying to become pregnant or considering pregnancy can be tested and/or monitored for presence and/or levels of auto-reactive antibodies that bind to a target expressed by an embryo or fetus (see 411).

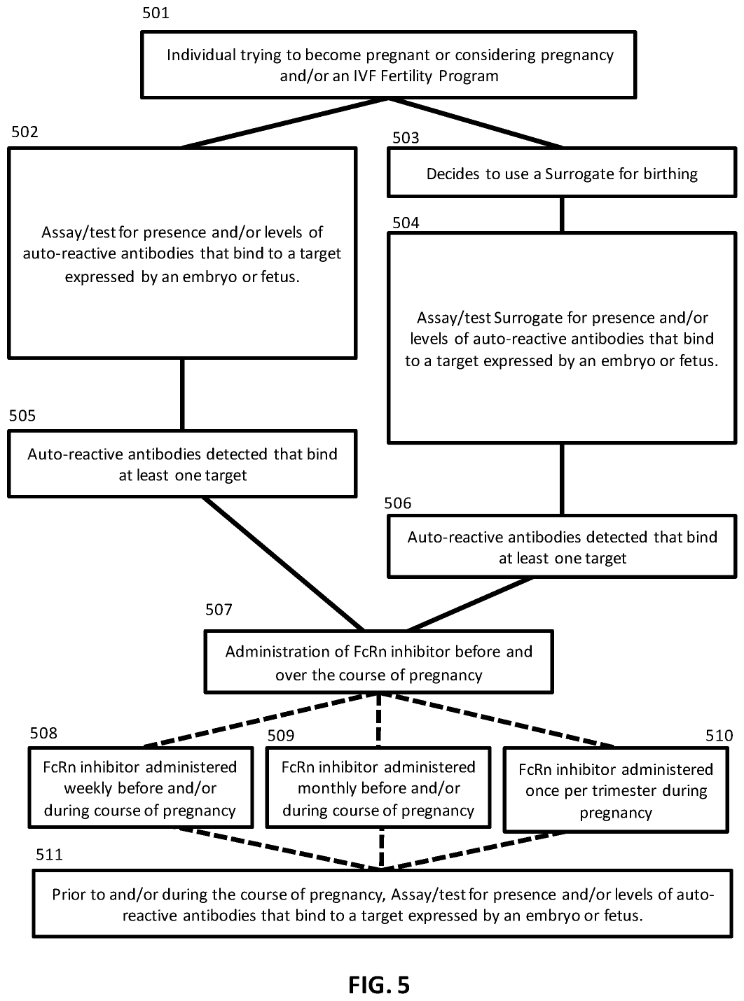

FIG. 5 illustrates a schematic for clinical diagnosis and administration of an FcRn inhibitor to an individual trying to become pregnant, considering pregnancy, or an individual serving as a surrogate. The individual trying to become pregnant, considering pregnancy, or an individual serving as a surrogate can be assayed or tested for the presence of auto-reactive antibodies (see, e.g., 502 and/or 504). In 502 and/or 504, A sample from a pregnant individual can be assayed for the presence of maternal autoantibodies that bind to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In some cases, it is known that the trying to become pregnant, considering pregnancy, or an individual serving as a surrogate has birthed at least one child diagnosed with ASD (503). The presence of at least one maternal autoantibody in the assay of 502 and/or 504 would result in the clinical decision to administer the FcRn inhibitor before and/or during pregnancy (307). Schedules for the administration of the FcRn inhibitor can be designed wherein the FcRn is administered weekly before pregnancy, monthly before pregnancy, weekly during pregnancy, monthly during pregnancy, and/or once per trimester (see 508, 509, and 510, respectively; dotted line indicates the option of more than one schedule available for administration). Prior to and/or during the course of pregnancy and/or following the administration of the FcRn inhibitor, the individual trying to become pregnant, considering pregnancy, or an individual serving as a surrogate can be tested and/or monitored for presence and/or levels of auto-reactive antibodies that bind to a target expressed by an embryo or fetus (see 511).

In some embodiments, the auto-reactive antibodies comprise antibodies that bind a central nervous system (CNS) target. In certain embodiments, an embryo or fetus expresses the CNS target. In some embodiments, the auto-reactive antibodies bind to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In certain embodiments, the auto-reactive antibodies bind to the target LDH-A. In certain embodiments, the auto-reactive antibodies bind to the target LDH-B. In certain embodiments, the auto-reactive antibodies bind to the target GDA. In certain embodiments, the auto-reactive antibodies bind to the target CRMP1. In certain embodiments, the auto-reactive antibodies bind to the target STIP1. In certain embodiments, the auto-reactive antibodies bind to the target DPYSL2. In certain embodiments, the auto-reactive antibodies bind to the target YBX1. In certain embodiments, the auto-reactive antibodies bind to the targetNSE. In certain embodiments, the auto-reactive antibodies bind to the target Caspr2. In certain embodiments, the auto-reactive antibodies bind to the target KCNAB2. In certain embodiments, the auto-reactive antibodies bind to the target KCNAB1. In certain embodiments, the auto-reactive antibodies bind to the target EDIL3. In certain embodiments, the auto-reactive antibodies bind to the target IVD. In certain embodiments, the auto-reactive antibodies bind to the target SUL4A1. In certain embodiments, the auto-reactive antibodies bind to the target TNIP2. In certain embodiments, the auto-reactive antibodies bind to the target RAI16. In certain embodiments, the auto-reactive antibodies bind to the target GDP D5. In some embodiments, the auto-reactive antibodies bind a neural antigen expressed by a fetus.

In some embodiments, the inhibitor of FcRn function prevents and/or blocks auto-reactive antibodies from contacting a fetus or embryo. In certain embodiments, the inhibitor of FcRn function reduces the number of autoreactive antibodies that contact an embryo or fetus. In certain embodiments, the inhibitor of FcRn function inhibits or prevents maternal autoreactive antibodies from crossing the placental barrier. In certain embodiments, the inhibitor of FcRn function inhibits or prevents maternal autoreactive antibodies from crossing the syncytiotrophoblast cell barrier. In certain embodiments, the inhibitor of FcRn function inhibits or prevents maternal autoreactive antibodies from crossing the cytotrophoblast cell barrier. In certain embodiments, the inhibitor of FcRn function inhibits or prevents maternal autoreactive antibodies from crossing the villous stroma.

In some embodiments, the inhibitor prevents an interaction between FcRn and an immunoglobulin G (IgG) molecule. In some embodiments, the inhibitor prevents FcRn-mediated rescue of an IgG molecule. In certain embodiments, the inhibitor prevents an interaction between FcRn and a human serum albumin molecule.

In some embodiments, the inhibitor comprises an antibody or a target-binding fragment thereof, a small molecule, a peptide, or a polypeptide, or a nucleic acid. In some embodiments, the inhibitor is an antibody or target binding fragment thereof. In certain embodiments, the antibody or the target-binding fragment thereof binds FcRn. In certain embodiments, the antibody, or target-binding fragment thereof, is selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof. In certain embodiments, the antibody is Rozanolixizumab. In certain embodiments, the antibody is SYNT001. In certain embodiments, the antibody M281. In certain embodiments, the antibody is Argx-113. In certain embodiments, the antibody is HL161-11G. In certain embodiments, the antibody is DX-2504. In certain embodiments, the antibody is DX-2507. In certain embodiments, the antibody is ABY039,IMVT-1401/RVT1401. In certain embodiments, the antibody or the target-binding fragment thereof comprises a complementarity-determining region of an antibody selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof.

In some embodiments, the inhibitor of FcRn function is a small molecule. In certain embodiments, the small molecule is selected from TABLE 1. In some embodiments, the inhibitor of FcRn function is a peptide inhibitor. In certain embodiments, the peptide is selected from TABLE 2. In some embodiments, the inhibitor of FcRn function is a protein. In certain embodiments, the protein comprises an Fc region of an IgG molecule. In certain embodiments, the Fc region of an IgG molecule comprises one or more mutations that increase its affinity to FcRn relative to a wild-type Fc molecule.

In some embodiments, the method further comprises comprising assaying a biological sample obtained from the pregnant individual or the individual trying to become pregnant or individual considering pregnancy, or an individual serving as a surrogate for the pregnancy, to detect a maternal antibody that binds to a target selected from the group consisting of a lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In certain embodiments, the biological sample is selected from the group consisting of a maternal blood plasma sample, a maternal blood serum sample, saliva, amniotic fluid, cord blood plasma, cord blood serum sample, fetal blood plasma, fetal blood serum, or a tissue sample. In certain embodiments, a biological sample obtained the pregnant individual or the individual trying to become pregnant or individual considering pregnancy, or an individual serving as a surrogate for the pregnancy, comprises a maternal antibody that binds to a fetal neural antigen. In certain embodiments, the biological sample is a blood plasma sample. In certain embodiments, the biological sample is a blood serum sample. In certain embodiments, the biological sample is a tissue sample.

In some embodiments, the pregnant individual is greater than 30 years old. In some embodiments, the pregnant individual is greater than 35 years old. In some embodiments, the pregnant individual is greater than 40 years old. In some embodiments, the pregnant individual is greater than 45 years old. In some embodiments, the pregnant individual or individual trying to become pregnant has birthed at least one child diagnosed with autism or an autism spectrum disorder.

In some embodiments, a sperm that fertilized an egg of the pregnant individual is from a person greater than 30 years old. In some embodiments, a sperm that fertilized an egg of the pregnant individual is from a person greater than 35 years old. In some embodiments, a sperm that fertilized an egg of the pregnant individual is from a person greater than 40 years old. In some embodiments, a sperm that fertilized an egg of the pregnant individual is from a person greater than 45 years old.

In some embodiments, administering is performed more than once over the course of a pregnancy of the pregnant individual. In some embodiments, administering is performed at least once during each trimester of a pregnancy. In some embodiments, the administering is performed during the first trimester. In some embodiments, the administering is performed during the second trimester. In some embodiments, the administering is performed during the second trimester. In some embodiments, the administering is performed during the third trimester. In some embodiments, the administering is performed during the second trimester. In some embodiments, the administering is performed during the third trimester. In some embodiments, the administering is performed daily. In some embodiments, the administering is performed weekly. In some embodiments, the administering is performed monthly.

In some embodiments, the inhibitor is administered to the pregnant individual before about 30, 60, 90, or 100 days post-conception. In some embodiments, the inhibitor is administered to the pregnant individual after about 100, 150, or 200 days post-conception.

In some embodiments, a gestational age of a fetus of the pregnant individual is less than about 30, 60, 90, or 100 days. In some embodiments, a gestational age of a fetus of the pregnant individual is greater than about 100, 150, or 200 days.

In some embodiments, reducing auto-reactive antibodies in the pregnant individual or the individual trying to become pregnant prevents or reduces symptoms associated with autism or an autism spectrum disorder. In some embodiments, reducing auto-reactive antibodies in the pregnant individual or the individual trying to become pregnant prevents or reduces symptoms associated with maternal autoantibody related autism spectrum disorder.

Further disclosed herein, is a method of reducing auto-reactive antibodies in a pregnant individual or an individual trying to become pregnant comprising: (a) obtaining a biological sample obtained from a pregnant individual or an individual considering or trying to become pregnant; (b) determining a presence of a maternal antibody that binds to a fetal neural antigen by at least one assay; (c) administering an inhibitor of neonatal Fc receptor (FcRn) function to the pregnant individual or the individual considering or trying to become pregnant when the presence of the maternal antibody that binds to the fetal neural antigen by the at least one assay is determined.

In some embodiments, the auto-reactive antibodies comprise antibodies that bind a central nervous system (CNS) target. In certain embodiments, an embryo or fetus expresses the CNS target. In some embodiments, the auto-reactive antibodies bind to a target selected from the group consisting of lactate dehydrogenase A (LDH-A), lactate dehydrogenase B (LDH-B), guanine deaminase (GDA), collapsin response mediator protein 1 (CRMP1), stress-induced phosphoprotein 1 (STIP1), a dihydropyrimidinase-like protein 2 (DPYSL2, CRIMP2) protein, a Y Box Binding Protein 1 (YBX1) protein, non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, and any combination thereof. In certain embodiments, the auto-reactive antibodies bind to the target LDH-A. In certain embodiments, the auto-reactive antibodies bind to the target LDH-B. In certain embodiments, the auto-reactive antibodies bind to the target GDA. In certain embodiments, the auto-reactive antibodies bind to the target CRMP1. In certain embodiments, the auto-reactive antibodies bind to the target STIP1. In certain embodiments, the auto-reactive antibodies bind to the target DPYSL2. In certain embodiments, the auto-reactive antibodies bind to the target YBX1. In certain embodiments, the auto-reactive antibodies bind to the targetNSE. In certain embodiments, the auto-reactive antibodies bind to the target Caspr2. In certain embodiments, the auto-reactive antibodies bind to the target KCNAB2. In certain embodiments, the auto-reactive antibodies bind to the target KCNAB1. In certain embodiments, the auto-reactive antibodies bind to the target EDIL3. In certain embodiments, the auto-reactive antibodies bind to the target IVD. In certain embodiments, the auto-reactive antibodies bind to the target SUL4A1. In certain embodiments, the auto-reactive antibodies bind to the target TNIP2. In certain embodiments, the auto-reactive antibodies bind to the target RAI16. In certain embodiments, the auto-reactive antibodies bind to the target GDP D5. In some embodiments, the auto-reactive antibodies bind a neural antigen expressed by a fetus.

In some embodiments, the assay is an immune assay. In some embodiments, the biological sample is selected from the group consisting of a maternal blood plasma sample, a maternal blood serum sample, saliva, amniotic fluid, cord blood plasma, cord blood serum sample, fetal blood plasma, fetal blood serum, or a tissue sample. In certain embodiments, a biological sample obtained the pregnant individual or the individual trying to become pregnant or individual considering pregnancy, or an individual serving as a surrogate for the pregnancy, comprises a maternal antibody that binds to a fetal neural antigen. In certain embodiments, the biological sample is a blood plasma sample. In certain embodiments, the biological sample is a blood serum sample. In certain embodiments, the biological sample is a tissue sample.

In some embodiments, the assay detects the presence and/or levels of a maternal antibody that binds to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In certain embodiments, the auto-reactive antibodies bind to the target LDH-A. In certain embodiments, the maternal antibody binds to the target LDH-B. In certain embodiments, the maternal antibody binds to the target GDA. In certain embodiments, the maternal antibody binds to the target CRMP1. In certain embodiments, the maternal antibody binds to the target STIP1. In certain embodiments, the maternal antibody binds to the target DPYSL2. In certain embodiments, the maternal antibody binds to the target YBX1. In certain embodiments, the maternal antibody binds to the target NSE. In certain embodiments, the maternal antibody binds to the target Caspr2. In certain embodiments, the maternal antibody binds to the target KCNAB2. In certain embodiments, the maternal antibody binds to the target KCNAB1. In certain embodiments, the maternal antibody binds to the target EDIL3. In certain embodiments, the maternal antibody binds to the target IVD. In certain embodiments, the maternal antibody binds to the target SUL4A1. In certain embodiments, the maternal antibody binds to the target TNIP2. In certain embodiments, the maternal antibody binds to the target RAI16. In certain embodiments, the maternal antibody binds to the target GDP D5. In some embodiments, the maternal antibody binds a neural antigen expressed by a fetus.

In some embodiments, the inhibitor of FcRn function prevents and/or blocks auto-reactive antibodies from contacting a fetus or embryo. In certain embodiments, the inhibitor of FcRn function reduces the number of autoreactive antibodies that contact an embryo or fetus. In certain embodiments, the inhibitor of FcRn function inhibits or prevents maternal autoreactive antibodies from crossing the placental barrier. In certain embodiments, the inhibitor of FcRn function inhibits or prevents maternal autoreactive antibodies from crossing the syncytiotrophoblast cell barrier. In certain embodiments, the inhibitor of FcRn function inhibits or prevents maternal autoreactive antibodies from crossing the cytotrophoblast cell barrier. In certain embodiments, the inhibitor of FcRn function inhibits or prevents maternal autoreactive antibodies from crossing the villous stroma.

In some embodiments, the inhibitor prevents an interaction between FcRn and an immunoglobulin G (IgG) molecule. In some embodiments, the inhibitor prevents FcRn-mediated rescue of an IgG molecule. In certain embodiments, the inhibitor prevents an interaction between FcRn and a human serum albumin molecule.

In some embodiments, the inhibitor comprises an antibody or a target-binding fragment thereof, a small molecule, a peptide, or a polypeptide, or a nucleic acid. In some embodiments, the inhibitor is an antibody or target binding fragment thereof. In certain embodiments, the antibody or the target-binding fragment thereof binds FcRn. In certain embodiments, the antibody, or target-binding fragment thereof, is selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof. In certain embodiments, the antibody is Rozanolixizumab. In certain embodiments, the antibody is SYNT001. In certain embodiments, the antibody M281. In certain embodiments, the antibody is Argx-113. In certain embodiments, the antibody is HL161-11G. In certain embodiments, the antibody is DX-2504. In certain embodiments, the antibody is DX-2507. In certain embodiments, the antibody is ABY039,IMVT-1401/RVT1401. In certain embodiments, the antibody or the target-binding fragment thereof comprises a complementarity-determining region of an antibody selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof.

In some embodiments, the inhibitor of FcRn function is a small molecule. In certain embodiments, the small molecule is selected from TABLE 1. In some embodiments, the inhibitor of FcRn function is a peptide inhibitor. In certain embodiments, the peptide is selected from TABLE 2. In some embodiments, the inhibitor of FcRn function is a protein. In certain embodiments, the protein comprises an Fc region of an IgG molecule. In certain embodiments, the Fc region of an IgG molecule comprises one or more mutations that increase its affinity to FcRn relative to a wild-type Fc molecule.

In some embodiments, the pregnant individual is greater than 30 years old. In some embodiments, the pregnant individual is greater than 35 years old. In some embodiments, the pregnant individual is greater than 40 years old. In some embodiments, the pregnant individual is greater than 45 years old. In some embodiments, the pregnant individual or individual trying to become pregnant has birthed at least one child diagnosed with autism or an autism spectrum disorder.

In some embodiments, a sperm that fertilized an egg of the pregnant individual is from a person greater than 30 years old. In some embodiments, a sperm that fertilized an egg of the pregnant individual is from a person greater than 35 years old. In some embodiments, a sperm that fertilized an egg of the pregnant individual is from a person greater than 40 years old. In some embodiments, a sperm that fertilized an egg of the pregnant individual is from a person greater than 45 years old.

In some embodiments, administering is performed more than once over the course of a pregnancy of the pregnant individual. In some embodiments, administering is performed at least once during each trimester of a pregnancy. In some embodiments, the administering is performed during the first trimester. In some embodiments, the administering is performed during the second trimester. In some embodiments, the administering is performed during the second trimester. In some embodiments, the administering is performed during the third trimester. In some embodiments, the administering is performed during the second trimester. In some embodiments, the administering is performed during the third trimester. In some embodiments, the administering is performed daily. In some embodiments, the administering is performed weekly. In some embodiments, the administering is performed monthly.

In some embodiments, the inhibitor is administered to the pregnant individual before about 30, 60, 90, or 100 days post-conception. In some embodiments, the inhibitor is administered to the pregnant individual after about 100, 150, or 200 days post-conception.

In some embodiments, a gestational age of a fetus of the pregnant individual is less than about 30, 60, 90, or 100 days. In some embodiments, a gestational age of a fetus of the pregnant individual is greater than about 100, 150, or 200 days.

In some embodiments, reducing auto-reactive antibodies in the pregnant individual or the individual trying to become pregnant prevents or reduces symptoms associated with autism or an autism spectrum disorder. In some embodiments, reducing auto-reactive antibodies in the pregnant individual or the individual trying to become pregnant prevents or reduces symptoms associated with maternal autoantibody related autism spectrum disorder.

Further disclosed herein, is the use of an inhibitor of neonatal Fc receptor (FcRn) function in a method for reducing auto-reactive antibodies in a pregnant individual or an individual considering or trying to become pregnant. Also disclosed herein, is the use of an inhibitor of neonatal Fc receptor (FcRn) function in the manufacture of a medicament for the treatment of auto-reactive antibodies in a pregnant individual or an individual trying to become pregnant.

### EXEMPLARY EMBODIMENTS

Provided herein are methods of reducing auto-reactive antibodies (e.g., an amount present in blood or sera) in a pregnant individual or an individual trying to become or an individual considering pregnancy, or an individual serving as a surrogate for the pregnancy, comprising administering, to the pregnant individual or individual trying to become pregnant or individual considering pregnancy, or an individual serving as a surrogate for the pregnancy, an inhibitor of neonatal Fc receptor (FcRn) function.

In some embodiments, the auto-reactive antibodies comprise antibodies that bind a central nervous system (CNS) target. In certain embodiments, an embryo or fetus expresses the CNS target. In some embodiments, the auto-reactive antibodies bind to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In certain embodiments, the auto-reactive antibodies bind to the target LDH-A. In certain embodiments, the auto-reactive antibodies bind to the target LDH-B. In certain embodiments, the auto-reactive antibodies bind to the target GDA. In certain embodiments, the auto-reactive antibodies bind to the target CRMP1. In certain embodiments, the auto-reactive antibodies bind to the target STIP1. In certain embodiments, the auto-reactive antibodies bind to the target DPYSL2. In certain embodiments, the auto-reactive antibodies bind to the target YBX1. In certain embodiments, the auto-reactive antibodies bind to the targetNSE. In certain embodiments, the auto-reactive antibodies bind to the target Caspr2. In certain embodiments, the auto-reactive antibodies bind to the target KCNAB2. In certain embodiments, the auto-reactive antibodies bind to the target KCNAB1. In certain embodiments, the auto-reactive antibodies bind to the target EDIL3. In certain embodiments, the auto-reactive antibodies bind to the target IVD. In certain embodiments, the auto-reactive antibodies bind to the target SUL4A1. In certain embodiments, the auto-reactive antibodies bind to the target TNIP2. In certain embodiments, the auto-reactive antibodies bind to the target RAI16. In certain embodiments, the auto-reactive antibodies bind to the target GDP D5. In some embodiments, the auto-reactive antibodies bind a neural antigen expressed by a fetus.

In some embodiments, the inhibitor of FcRn function prevents and/or blocks auto-reactive antibodies from contacting a fetus or embryo. In certain embodiments, the inhibitor of FcRn function reduces the number of autoreactive antibodies that contact an embryo or fetus. In certain embodiments, the inhibitor of FcRn function inhibits or prevents maternal autoreactive antibodies from crossing the placental barrier. In certain embodiments, the inhibitor of FcRn function inhibits or prevents maternal autoreactive antibodies from crossing the syncytiotrophoblast cell barrier. In certain embodiments, the inhibitor of FcRn function inhibits or prevents maternal autoreactive antibodies from crossing the cytotrophoblast cell barrier. In certain embodiments, the inhibitor of FcRn function inhibits or prevents maternal autoreactive antibodies from crossing the villous stroma.

In some embodiments, the inhibitor prevents an interaction between FcRn and an immunoglobulin G (IgG) molecule. In some embodiments, the inhibitor prevents FcRn-mediated rescue of an IgG molecule. In certain embodiments, the inhibitor prevents an interaction between FcRn and a human serum albumin molecule.

In some embodiments, the inhibitor comprises an antibody or a target-binding fragment thereof, a small molecule, a peptide, or a polypeptide, or a nucleic acid. In some embodiments, the inhibitor is an antibody or target binding fragment thereof. In certain embodiments, the antibody or the target-binding fragment thereof binds FcRn. In certain embodiments, the antibody, or target-binding fragment thereof, is selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof. In certain embodiments, the antibody is Rozanolixizumab. In certain embodiments, the antibody is SYNT001. In certain embodiments, the antibody M281. In certain embodiments, the antibody is Argx-113. In certain embodiments, the antibody is HL161-11G. In certain embodiments, the antibody is DX-2504. In certain embodiments, the antibody is DX-2507. In certain embodiments, the antibody is ABY039,IMVT-1401/RVT1401. In certain embodiments, the antibody or the target-binding fragment thereof comprises a complementarity-determining region of an antibody selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof.

In some embodiments, the inhibitor of FcRn function comprises an antibody-binding domain region (e.g., a polypeptide comprising an epitope or an antibody variable domain that binds the Fc region of another antibody) and a variant Fc region, or FcRn-binding fragment thereof, wherein Fc domain of the Fc region or FcRn-binding fragment thereof comprise one or more amino acid substitutions that increase binding to FcRn relative to a wildtype immunoglobulin Fc region. In some embodiments, the inhibitor of FcRn function is a polypeptide comprising a variant Fc region. In certain embodiments, the variant Fc region, or FcRn-binding fragment thereof, comprising the one or more amino acid substitutions that increase binding FcRn (e.g., as described herein) relative to a wild-type IgG1 Fc region. In certain embodiments, the one or more amino acid substitutions comprises Y, T, E, K, F, and Y at EU positions 252, 254, 256, 433, 434, and 436 respectively, and wherein the Fc region binds to FcRn with increased affinity and reduced pH dependence relative to a wild-type IgG1 Fc region. In some embodiments, the inhibitor of FcRn function is an antibody comprising a variant Fc region, wherein the antibody (e.g., an antibody that inhibits FcRn function) comprises a variable domain that recognizes the Fc region of an antibody. In some embodiments, the inhibitor of FcRn function is a fusion polypeptide comprising a polypeptide and a variant Fc region. In certain embodiments, the polypeptide comprises an epitope specific to the auto-reactive antibody.

In some embodiments, the inhibitor of FcRn function is a small molecule. In certain embodiments, the small molecule is selected from TABLE 1. In some embodiments, the inhibitor of FcRn function is a peptide inhibitor. In certain embodiments, the peptide is selected from TABLE 2. In some embodiments, the inhibitor of FcRn function is a protein. In certain embodiments, the protein comprises an Fc region of an IgG molecule. In certain embodiments, the Fc region of an IgG molecule comprises one or more mutations that increase its affinity to FcRn relative to a wild-type Fc molecule.

In some embodiments, the inhibitor of FcRn function is a variant Fc molecule, or FcRn-binding fragment thereof, wherein the variant Fc region molecule or FcRn-binding fragment thereof comprise one or more amino acid substitutions that increase binding to FcRn relative to a wildtype immunoglobulin Fc region. In some embodiments, the variant Fc molecule is engineered to bind with increased affinity to FcRn through their Fc region at both acidic and near neutral pH. In some embodiments, the variant Fc molecule, or FcRn-binding fragment thereof, wherein the Fc domains of the Fc region or FcRn-binding fragment thereof comprise the amino acids Y, T, E, K, F, and Y at EU positions 252, 254, 256, 433, 434, and 436 respectively, and wherein the Fc region binds to FcRn with increased affinity and reduced pH dependence relative to a wild-type IgG1 Fc region. In some embodiments,

In some embodiments, the inhibitor of FcRn function is an antibody (e.g., an antibody that inhibits FcRn function) comprising an Fc-engineered antibody that enhances IgG degradation. In some embodiments the antibody is engineered to bind with increased affinity to FcRn through their Fc region at both acidic and near neutral pH. In some embodiments, the antibody (e.g., an antibody that inhibits FcRn function) comprises a variant Fc region, or FcRn-binding fragment thereof, wherein the Fc domains of the Fc region or FcRn-binding fragment thereof comprise the amino acids Y, T, E, K, F, and Y at EU positions 252, 254, 256, 433, 434, and 436 respectively, and wherein the Fc region binds to FcRn with increased affinity and reduced pH dependence relative to a wild-type IgG1 Fc region.

In some embodiments, the method further comprises comprising assaying a biological sample obtained from the pregnant individual or the individual trying to become pregnant or individual considering pregnancy, or an individual serving as a surrogate for the pregnancy, to detect a maternal antibody that binds to a target selected from the group consisting of a lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In some embodiments, the maternal autoantibody is LDHA, LDHB, CRMP1, STIP1, or any combination thereof. In some embodiments, the maternal autoantibody is LDHA. In some embodiments, the maternal autoantibody is LDHB. In some embodiments, the maternal autoantibody is CRMP1. In some embodiments, the maternal autoantibody is STIP1 In some embodiments, the maternal autoantibody is any combination thereof LDHA, LDHB, CRMP1, and/or STIP1. In some embodiments, the maternal autoantibody is CRMP1 and STIP1,

In certain embodiments, the biological sample is selected from the group consisting of a maternal blood plasma sample, a maternal blood serum sample, saliva, amniotic fluid, cord blood plasma, cord blood serum sample, fetal blood plasma, fetal blood serum, or a tissue sample. In certain embodiments, a biological sample obtained the pregnant individual or the individual trying to become pregnant or individual considering pregnancy, or an individual serving as a surrogate for the pregnancy, comprises a maternal antibody that binds to a fetal neural antigen. In certain embodiments, the biological sample is a blood plasma sample. In certain embodiments, the biological sample is a blood serum sample. In certain embodiments, the biological sample is a tissue sample.

In some embodiments, the pregnant individual is greater than 30 years old. In some embodiments, the pregnant individual is greater than 35 years old. In some embodiments, the pregnant individual is greater than 40 years old. In some embodiments, the pregnant individual is greater than 45 years old. In some embodiments, the pregnant individual or individual trying to become pregnant has birthed at least one child diagnosed with autism or an autism spectrum disorder.

In some embodiments, a sperm that fertilized an egg of the pregnant individual is from a person greater than 30 years old. In some embodiments, a sperm that fertilized an egg of the pregnant individual is from a person greater than 35 years old. In some embodiments, a sperm that fertilized an egg of the pregnant individual is from a person greater than 40 years old. In some embodiments, a sperm that fertilized an egg of the pregnant individual is from a person greater than 45 years old.

In some embodiments, administering is performed more than once over the course of a pregnancy of the pregnant individual. In some embodiments, administering is performed at least once during each trimester of a pregnancy. In some embodiments, the administering is performed during the first trimester. In some embodiments, the administering is performed during the second trimester. In some embodiments, the administering is performed during the second trimester. In some embodiments, the administering is performed during the third trimester. In some embodiments, the administering is performed during the second trimester. In some embodiments, the administering is performed during the third trimester. In some embodiments, the administering is performed daily. In some embodiments, the administering is performed weekly. In some embodiments, the administering is performed monthly.

In some embodiments, the inhibitor is administered to the pregnant individual before about 30, 60, 90, or 100 days post-conception. In some embodiments, the inhibitor is administered to the pregnant individual after about 100, 150, or 200 days post-conception.

In some embodiments, a gestational age of a fetus of the pregnant individual is less than about 30, 60, 90, or 100 days. In some embodiments, a gestational age of a fetus of the pregnant individual is greater than about 100, 150, or 200 days.

In some embodiments, reducing auto-reactive antibodies in the pregnant individual or the individual trying to become pregnant prevents or reduces symptoms associated with autism or an autism spectrum disorder. In some embodiments, reducing auto-reactive antibodies in the pregnant individual or the individual trying to become pregnant prevents or reduces symptoms associated with maternal autoantibody related autism spectrum disorder.

Further disclosed herein, are methods of reducing auto-reactive antibodies in a pregnant individual or an individual trying to become pregnant comprising: (a) obtaining a biological sample obtained from a pregnant individual or an individual considering or trying to become pregnant; (b) determining a presence of a maternal antibody that binds to a fetal neural antigen by at least one assay; (c) administering an inhibitor of neonatal Fc receptor (FcRn) function to the pregnant individual or the individual considering or trying to become pregnant when the presence of the maternal antibody that binds to the fetal neural antigen by the at least one assay is determined.

In some embodiments, the auto-reactive antibodies comprise antibodies that bind a central nervous system (CNS) target. In certain embodiments, an embryo or fetus expresses the CNS target. In some embodiments, the auto-reactive antibodies bind to a target selected from the group consisting of lactate dehydrogenase A (LDH-A), lactate dehydrogenase B (LDH-B), guanine deaminase (GDA), collapsin response mediator protein 1 (CRMP1), stress-induced phosphoprotein 1 (STIP1), a dihydropyrimidinase-like protein 2 (DPYSL2, CRIMP2) protein, a Y Box Binding Protein 1 (YBX1) protein, non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, and any combination thereof. In certain embodiments, the auto-reactive antibodies bind to the target LDH-A. In certain embodiments, the auto-reactive antibodies bind to the target LDH-B. In certain embodiments, the auto-reactive antibodies bind to the target GDA. In certain embodiments, the auto-reactive antibodies bind to the target CRMP1. In certain embodiments, the auto-reactive antibodies bind to the target STIP1. In certain embodiments, the auto-reactive antibodies bind to the target DPYSL2. In certain embodiments, the auto-reactive antibodies bind to the target YBX1. In certain embodiments, the auto-reactive antibodies bind to the targetNSE. In certain embodiments, the auto-reactive antibodies bind to the target Caspr2. In certain embodiments, the auto-reactive antibodies bind to the target KCNAB2. In certain embodiments, the auto-reactive antibodies bind to the target KCNAB1. In certain embodiments, the auto-reactive antibodies bind to the target EDIL3. In certain embodiments, the auto-reactive antibodies bind to the target IVD. In certain embodiments, the auto-reactive antibodies bind to the target SUL4A1. In certain embodiments, the auto-reactive antibodies bind to the target TNIP2. In certain embodiments, the auto-reactive antibodies bind to the target RAI16. In certain embodiments, the auto-reactive antibodies bind to the target GDP D5. In some embodiments, the auto-reactive antibodies bind a neural antigen expressed by a fetus.

In some embodiments, the assay is an immune assay. In some embodiments, the biological sample is selected from the group consisting of a maternal blood plasma sample, a maternal blood serum sample, saliva, amniotic fluid, cord blood plasma, cord blood serum sample, fetal blood plasma, fetal blood serum, or a tissue sample. In certain embodiments, a biological sample obtained the pregnant individual or the individual trying to become pregnant or individual considering pregnancy, or an individual serving as a surrogate for the pregnancy, comprises a maternal antibody that binds to a fetal neural antigen. In certain embodiments, the biological sample is a blood plasma sample. In certain embodiments, the biological sample is a blood serum sample. In certain embodiments, the biological sample is a tissue sample.

In some embodiments, the assay detects the presence and/or levels of a maternal antibody that binds to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. In certain embodiments, the auto-reactive antibodies bind to the target LDH-A. In certain embodiments, the maternal antibody binds to the target LDH-B. In certain embodiments, the maternal antibody binds to the target GDA. In certain embodiments, the maternal antibody binds to the target CRMP1. In certain embodiments, the maternal antibody binds to the target STIP1. In certain embodiments, the maternal antibody binds to the target DPYSL2. In certain embodiments, the maternal antibody binds to the target YBX1. In certain embodiments, the maternal antibody binds to the target NSE. In certain embodiments, the maternal antibody binds to the target Caspr2. In certain embodiments, the maternal antibody binds to the target KCNAB2. In certain embodiments, the maternal antibody binds to the target KCNAB1. In certain embodiments, the maternal antibody binds to the target EDIL3. In certain embodiments, the maternal antibody binds to the target IVD. In certain embodiments, the maternal antibody binds to the target SUL4A1. In certain embodiments, the maternal antibody binds to the target TNIP2. In certain embodiments, the maternal antibody binds to the target RAI16. In certain embodiments, the maternal antibody binds to the target GDP D5. In some embodiments, the maternal antibody binds a neural antigen expressed by a fetus.

In some embodiments, the inhibitor of FcRn function prevents and/or blocks auto-reactive antibodies from contacting a fetus or embryo. In certain embodiments, the inhibitor of FcRn function reduces the number of autoreactive antibodies that contact an embryo or fetus. In certain embodiments, the inhibitor of FcRn function inhibits or prevents maternal autoreactive antibodies from crossing the placental barrier. In certain embodiments, the inhibitor of FcRn function inhibits or prevents maternal autoreactive antibodies from crossing the syncytiotrophoblast cell barrier. In certain embodiments, the inhibitor of FcRn function inhibits or prevents maternal autoreactive antibodies from crossing the cytotrophoblast cell barrier. In certain embodiments, the inhibitor of FcRn function inhibits or prevents maternal autoreactive antibodies from crossing the villous stroma.

In some embodiments, the inhibitor prevents an interaction between FcRn and an immunoglobulin G (IgG) molecule. In some embodiments, the inhibitor prevents FcRn-mediated rescue of an IgG molecule. In certain embodiments, the inhibitor prevents an interaction between FcRn and a human serum albumin molecule.

In some embodiments, the inhibitor comprises an antibody or a target-binding fragment thereof, a small molecule, a peptide, or a polypeptide, or a nucleic acid. In some embodiments, the inhibitor is an antibody or target binding fragment thereof. In certain embodiments, the antibody or the target-binding fragment thereof binds FcRn. In certain embodiments, the antibody, or target-binding fragment thereof, is selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof. In certain embodiments, the antibody is Rozanolixizumab. In certain embodiments, the antibody is SYNT001. In certain embodiments, the antibody M281. In certain embodiments, the antibody is Argx-113. In certain embodiments, the antibody is HL161-11G. In certain embodiments, the antibody is DX-2504. In certain embodiments, the antibody is DX-2507. In certain embodiments, the antibody is ABY039,IMVT-1401/RVT1401. In certain embodiments, the antibody or the target-binding fragment thereof comprises a complementarity-determining region of an antibody selected from the group consisting of Rozanolixizumab, SYNT001, M281, Argx-113, HL161-11G, HL161-11H, HL161-1A, DX-2504, DX-2507, ABY039,IMVT-1401/RVT1401, and any combination thereof.

In some embodiments, the inhibitor of FcRn function is a small molecule. In certain embodiments, the small molecule is selected from TABLE 1. In some embodiments, the inhibitor of FcRn function is a peptide inhibitor. In certain embodiments, the peptide is selected from TABLE 2. In some embodiments, the inhibitor of FcRn function is a protein. In certain embodiments, the protein comprises an Fc region of an IgG molecule. In certain embodiments, the Fc region of an IgG molecule comprises one or more mutations that increase its affinity to FcRn relative to a wild-type Fc molecule.

In some embodiments, the pregnant individual is greater than 30 years old. In some embodiments, the pregnant individual is greater than 35 years old. In some embodiments, the pregnant individual is greater than 40 years old. In some embodiments, the pregnant individual is greater than 45 years old. In some embodiments, the pregnant individual or individual trying to become pregnant has birthed at least one child diagnosed with autism or an autism spectrum disorder.

In some embodiments, a sperm that fertilized an egg of the pregnant individual is from a person greater than 30 years old. In some embodiments, a sperm that fertilized an egg of the pregnant individual is from a person greater than 35 years old. In some embodiments, a sperm that fertilized an egg of the pregnant individual is from a person greater than 40 years old. In some embodiments, a sperm that fertilized an egg of the pregnant individual is from a person greater than 45 years old.

In some embodiments, administering is performed more than once over the course of a pregnancy of the pregnant individual. In some embodiments, administering is performed at least once during each trimester of a pregnancy. In some embodiments, the administering is performed during the first trimester. In some embodiments, the administering is performed during the second trimester. In some embodiments, the administering is performed during the second trimester. In some embodiments, the administering is performed during the third trimester. In some embodiments, the administering is performed during the second trimester. In some embodiments, the administering is performed during the third trimester. In some embodiments, the administering is performed daily. In some embodiments, the administering is performed weekly. In some embodiments, the administering is performed monthly.

In some embodiments, the inhibitor is administered to the pregnant individual before about 30, 60, 90, or 100 days post-conception. In some embodiments, the inhibitor is administered to the pregnant individual after about 100, 150, or 200 days post-conception.

In some embodiments, a gestational age of a fetus of the pregnant individual is less than about 30, 60, 90, or 100 days. In some embodiments, a gestational age of a fetus of the pregnant individual is greater than about 100, 150, or 200 days.

In some embodiments, reducing auto-reactive antibodies in the pregnant individual or the individual trying to become pregnant prevents or reduces symptoms associated with autism or an autism spectrum disorder. In some embodiments, reducing auto-reactive antibodies in the pregnant individual or the individual trying to become pregnant prevents or reduces symptoms associated with maternal autoantibody related autism spectrum disorder.

Further disclosed herein, is the use of an inhibitor of neonatal Fc receptor (FcRn) function in methods for reducing auto-reactive antibodies in a pregnant individual or an individual considering or trying to become pregnant. Also disclosed herein, is the use of an inhibitor of neonatal Fc receptor (FcRn) function in the manufacture of a medicament for the treatment of auto-reactive antibodies in a pregnant individual or an individual trying to become pregnant.

As used herein, “a” or “an” when used in conjunction with the term “comprising” in the claims and/or the specification includes and/or refers to “one” and also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. As also used herein, in any instance or embodiment described herein, “comprising” may be replaced with “consisting essentially of” and/or “consisting of”. used herein, in any instance or embodiment described herein, “comprises” may be replaced with “consists essentially of” and/or “consists of”.

As used herein, the term “about” in the context of a given value or range includes and/or refers to a value or range that is within 10% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

### EXAMPLES

The following illustrative examples are representative of embodiments of compositions and methods described herein and are not meant to be limiting in any way.

### Example 1: Administration of an FcRn Inhibitors to Pregnant Individual

Female patient No. 1 (P1) confirms a positive pregnancy home test 3 days prior to visiting a physician and wherein the home test is confirmed in the physician's office by methods routine in the art. P1 indicates to the physician that she previously birthed at least 1 child diagnosed with ASD in a prior pregnancy. The physician proceeds to prescribe, to P1, an antibody that targets FcRn, wherein the antibody that targets FcRn is to be administered intravenously every week during the course of P1's pregnancy.

Female patient No. 2 (P2) confirms a positive pregnancy home test 3 days prior to visiting a physician and wherein the home test is confirmed in the physician's office by methods routine in the art. P2 indicates to the physician that she previously birthed at least 1 child diagnosed with ASD in a prior pregnancy. The physician proceeds to collect a serum sample from P2 to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P2 tests positive for at least one of the antigens in the panel. The physician proceeds to prescribe, to P2, an antibody that targets FcRn, wherein the antibody that targets FcRn is to be administered intravenously every two weeks during the course of P2's pregnancy.

Female patient No. 3 (P3) confirms a positive pregnancy home test 3 days prior to visiting a physician and wherein the home test is confirmed in the physician's office by methods routine in the art. P3 indicates to the physician that she had not previously birthed any children. The physician proceeds to collect a serum sample from P3 to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P3 tested positive for at least one of the antigens in the panel. The physician proceeds to prescribe, to P3, an antibody that targets FcRn, wherein the antibody that targets FcRn is to be administered intravenously every week during the course of P3's pregnancy.

Female patient No. 4 (P4) confirms a positive pregnancy home test 3 days prior to visiting a physician and wherein the home test is confirmed in the physician's office by methods routine in the art. P5 indicates to the physician that she had not previously birthed any children. The physician proceeds to collect a serum sample from P4 to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P4 did not test positive for at least one antigen in the panel. The physician recommends another test during P4's appointment 21 days after the initial appointment. P4 tested positive for at least one antigen in the panel. The physician proceeds to prescribe, to P4, an antibody that targets FcRn, wherein the antibody that targets FcRn is to be administered intravenously every week during the course of P4's pregnancy.

Female patient No. 5 (P5) confirms a positive pregnancy home test 3 days prior to visiting a physician and wherein the home test is confirmed in the physician's office by methods routine in the art. P5 indicates to the physician that she had not previously birthed any children. The physician proceeds to collect a serum sample from P5 to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P5 tested positive for at least one of the antigens in the panel. The physician proceeds to prescribe, to P5, an antibody that targets FcRn, wherein the antibody that targets FcRn is to be administered intravenously every week during the course of P3's pregnancy. The physician proceeds to collect a serum sample from P5 every month during P5's pregnancy to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P5 subsequently demonstrates a decrease in levels of antibodies binding at least one of the antigens tested after the initial month of administering the antibody that targets FcRn.

### Example 2: Administration of an FcRn Inhibitors to and Individual Trying to Become Pregnant or Considering Pregnancy

Female patient No. 6 (P6) visits a physician (e.g. a family physician, family planner, or genetic counselor). P6 indicates to the physician that she previously birthed at least 1 child diagnosed with ASD in a prior pregnancy. The physician proceeds to collect a serum sample from P6 to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P6 tested positive for at least one of the antigens in the panel. The physician proceeds to prescribe, to P6, an antibody that targets FcRn, wherein the antibody that targets FcRn is to be administered intravenously bi-weekly while P6 is actively trying to conceive. The physician proceeds to prescribe, to P6, an antibody that targets FcRn, wherein the antibody that targets FcRn is to be administered intravenously every week after P6 becomes pregnant, for the duration the course of P6's pregnancy.

Female patient No. 7 (P7) visits a physician (e.g. a family physician, family planner, or genetic counselor) wherein P7 plans to conceive or become pregnant via in vitro fertilization. P7 indicates to the physician that she previously birthed at least 1 child diagnosed with ASD in a prior pregnancy. The physician proceeds to collect a serum sample from P7 to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P7 tested positive for at least one of the antigens in the panel. The physician proceeds to prescribe, to P7, an antibody that targets FcRn, wherein the antibody that targets FcRn is to be administered intravenously weekly while P7 is actively trying to conceive. The physician proceeds to prescribe, to P7, an antibody that targets FcRn, wherein the antibody that targets FcRn is to be administered intravenously every week after P7 becomes pregnant, for the duration the course of P7's pregnancy.

Female patient No. 8 (P8) visits a physician (e.g. a family physician, family planner, or genetic counselor) wherein P8 plans to conceive or become pregnant via in vitro fertilization. P8 indicates to the physician that she had not previously birthed child diagnosed with ASD in a prior pregnancy. The physician proceeds to collect a serum sample from P8 to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P8 tested positive for at least one of the antigens in the panel. The physician proceeds to prescribe, to P8, an antibody that targets FcRn, wherein the antibody that targets FcRn is to be administered intravenously weekly while P8 is actively trying to conceive. The physician proceeds to prescribe, to P8, an antibody that targets FcRn, wherein the antibody that targets FcRn is to be administered intravenously every week after P8 becomes pregnant, for the duration the course of P8's pregnancy.

Female patient No. 9 (P9) visits a physician (e.g. a family physician, family planner, or genetic counselor). P9 indicates to the physician that she previously birthed at least 1 child diagnosed with ASD in a prior pregnancy. The physician proceeds to prescribe, to P9, an antibody that targets FcRn, wherein the antibody that targets FcRn is to be administered intravenously every week during the course of P9's pregnancy. The physician proceeds to prescribe, to P9, an antibody that targets FcRn, wherein the antibody that targets FcRn is to be administered intravenously bi-weekly while P9 is actively trying to conceive. The physician proceeds to prescribe, to P9, an antibody that targets FcRn, wherein the antibody that targets FcRn is to be administered intravenously every week after P9 becomes pregnant, for the duration the course of P9's pregnancy.

Female patient No. 10 (P10) visits a physician (e.g. a family physician, family planner, or genetic counselor). P10 indicates to the physician that she is planning to serve as a surrogate. The physician proceeds to collect a serum sample from P10 to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P10 does not test positive for any of the biomarkers in the panel.

Female patient No. 11 (P11) visits a physician (e.g. a family physician, family planner, or genetic counselor). P11 indicates to the physician that she is planning to serve as a surrogate. The physician proceeds to collect a serum sample from P11 to be tested for antibodies that bind at least one antigen in the panel consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof. P11 tests positive for one or more of the biomarkers in the panel. The physician proceeds to prescribe, to P11, an antibody that targets FcRn, wherein the antibody that targets FcRn is to be administered intravenously every week during the course of P11's pregnancy. The physician proceeds to prescribe, to P11, an antibody that targets FcRn, wherein the antibody that targets FcRn is to be administered intravenously bi-weekly while P9 is actively trying to conceive. The physician proceeds to prescribe, to P11, an antibody that targets FcRn, wherein the antibody that targets FcRn is to be administered intravenously every week after P9 becomes pregnant, for the duration the course of P11's pregnancy.

### Example 3: FcRn Inhibitors Reduce Auto-Reactive Antibodies In Vivo

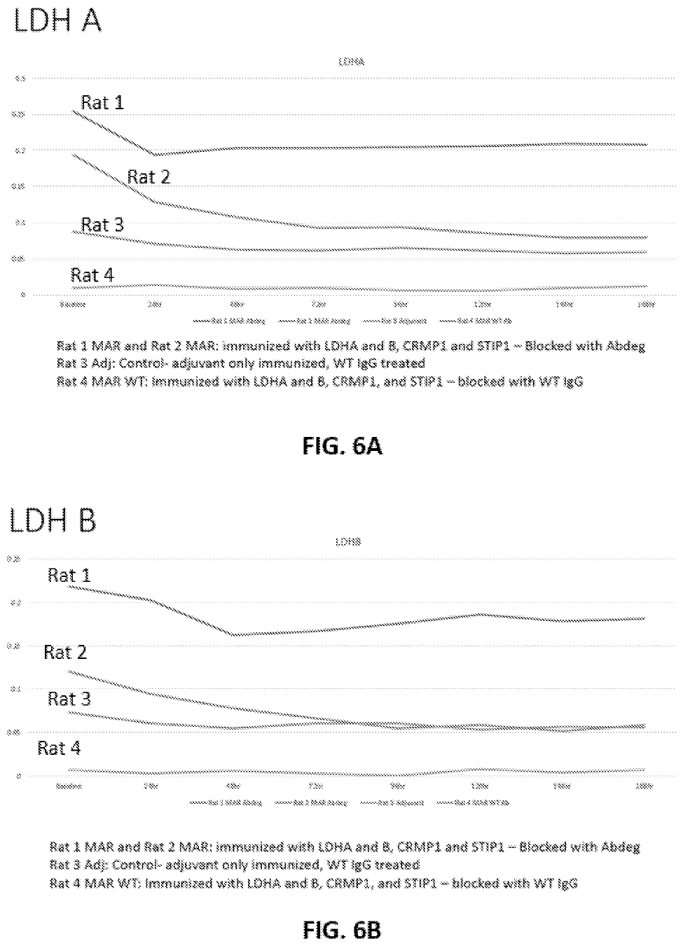

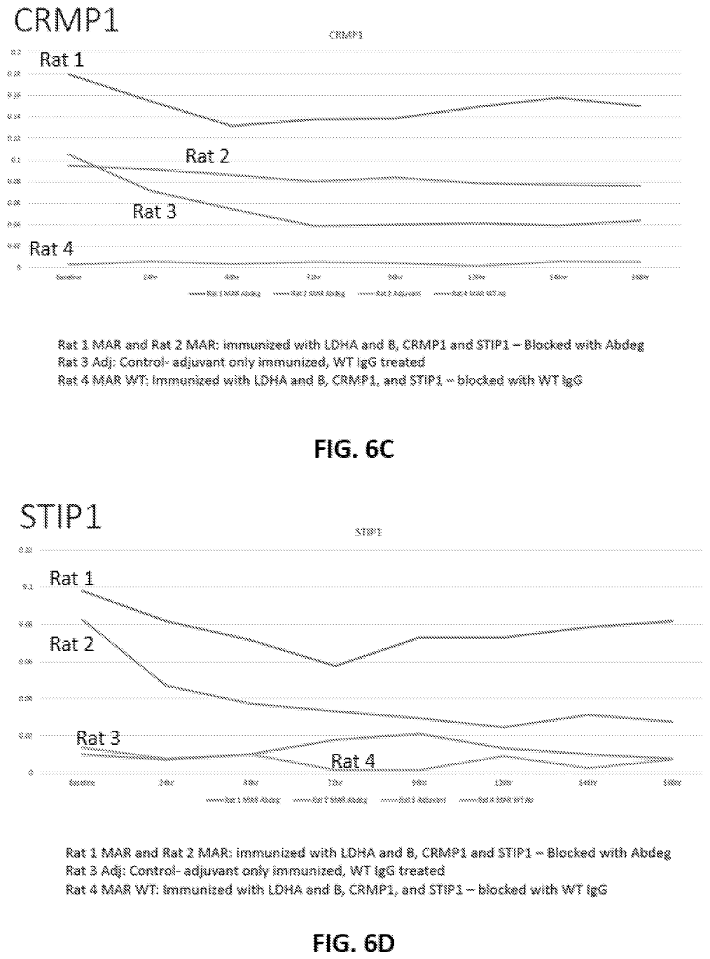

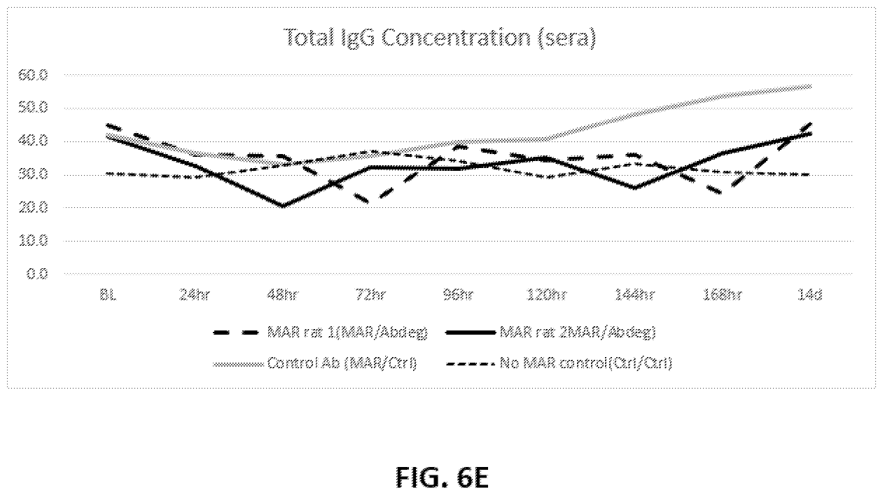

The test rats were immunized with LDHA and B, CRMP1, and STIP1 (referred to as MAR rat) and after ensuring that the rats had an IgG response to each of the proteins, two animals were treated with 1 IV injection of Abdeg (a variant Fc molecule having FcRn binding to FcRn compared to wt IgG, see e.g., SEQ ID NOs: 1-3) mutated antibody that modulates the concentrations of endogenous IgGs in vivo) at 5 mg/dose (provided from a stock solution ˜20 mg/mL). Baseline measurements for amount of LDHA, LDHB, CRMP1, or STIP1 antibodies were obtained prior to administering the test article. Post-administration measurements were obtained at 24 hours, 48 hours, 72 hours, 96 hours, 120 hours, 144 hours, and 160 hours. The control MAR positive animal was treated with a wild-type IgG. A control MAR negative animal treated with adjuvant only and wild-type IgG. The animals were bled at baseline and then daily for one week. Amount of LDHA, LDHB, CRMP1, or STIP1 antibodies were detected by absorbance via immunoassay. TABLE 1 shows absorbance data. FIGS. 6A-6E show plots for amount of LDHA, LDHB, CRMP1, STIP1, and total IgG. TABLE 1 and FIGS. 6A-6E show that auto-reactive antibodies that target LDHA, LDHB, CRMP1, and STIP1 are reduced even after a single administration of an inhibitor of neonatal Fc receptor (FcRn)function. Interestingly, an apparent reduction in total IgG was not discernable as shown in FIG. 6E.

TABLE 3
Reduction of auto-reactive antibodies

| | | Reduction in Absorbance (relative to baseline) | | | |
|---|---|---|---|---|---|
| | Subject | LDHA | LDHB | CRMP1 | STIP1 |
| 1 | MAR rat + Abdeg | 5.6% | 5.5% | 4.8% | 4.1% |
| 2 | MAR rat + Abdeg | 10.2% | 6.5% | 6.7% | 5.3% |
| 3 | MAR rat + control | 2.3% | 1.3% | 0.8% | 0.5% |

This reduction was seen even after a single administration and would be expected to be more dramatic with a plurality of doses. Additionally, the rats tested were specifically immunized increase levels of specific maternal autoreactive antibodies not normally seen at high levels in unimmunized mice, and thus the effect may be more pronounced in individuals that might have smaller increases in maternal autoreactive antibodies.

### Example 4: Pre-Clinical Study to Demonstrate the Ability of IV Anti-FcRn Antibodies in the Prevention of an ASD-Like Phenotype in a MAR Rat Model

The MAR rat model was designed to examine the effects of prenatal MAR antibody exposure on offspring neurodevelopment, and the remediation of these effects through the administration of IV anti-FcRn antibodies during gestation. Behavioral measures following newly established NIH guidelines (NOT-MH-19-053) are used and are selected to address scientific questions in the context of underlying brain processes and not for presumed congruence to human symptoms. Sophisticated social and cognitive repertoire testing of the rat model to evaluate behavioral changes for the 3 most clinically represented AB (antibody) patterns is planned for the study. The decision to focus on species-typical social development is based on previous findings from the mouse MAR model, as well as the Example 3 observations.

Antigen-driven Mouse Model: The identification of peptide epitope sequences on seven antigenic proteins targeted by ASD associated maternal ABs provided the opportunity to develop the first antigen-driven animal model, in which C57BL/6J mice were initiated. The AB pattern reflecting LDH-A, LDH-B, CRMP1 and STIP1 antigens was used. Whereas previous studies employed a passive antibody approach, the antigen-driven model administered specific peptide antigens to females prior to breeding to ensure a continuous exposure to the endogenously generated antibodies from conception through birth. Male and female offspring were tested using a comprehensive sequence of behavioral assays, as well as measures of health and development to evaluate the pathogenic significance of continued gestational exposure to MAR antibodies. Alterations in species-typical social behavior were observed during reciprocal social interactions in same-sex juvenile dyads and during adult male-female social interactions.

Juvenile MAR-ASD Mouse Behavior. MAR-ASD mice engaged in significantly fewer bouts of nose-nose sniffing, nose-anogenital sniffing, push-crawl play behavior, front approach behavior, and following behavior, indicating lower levels of social interactions relative to controls. Prenatal exposure to the MAR ABs also influenced repetitive self-grooming behaviors, as MAR-ASD juvenile mice engaged in significantly more repetitive self-grooming bouts compared to controls. There were no significant effects of offspring sex or treatment×sex interactions for any parameter (p >0.10).

Adult MAR-ASD Mouse Behavior. MAR-ASD adult males spent significantly less time engaging in nose-anogenital sniff, body sniff, front approach, and following behavior, and emitted significantly fewer total USVs over the 5-min test session. Thus, the first antigen-driven mouse model demonstrated that combined exposure to epitopes specific for LDH-A/B, STIP1 and CRMP1 generates mouse offspring with alterations in social and communication behavior and increased repetitive behaviors.

Antigen-driven Rat: Although mice have gained prominence in preclinical models, certain complex behaviors and physiological processes are difficult or impossible to model in these animals. This is due to specific brain regions being absent or less developed in mice, such as the prefrontal cortex; a region important for complex cognitive processing and social behavior. A study was conducted in rats to determine feasibility of establishing an antigen-driven MAR rat model. Rat dams underwent 4 weeks of subcutaneous (SubQ) injection with MAR ASD-specific antigens from the original pattern (LDH-A/B, STIP1, CRMP1) along with Freund's adjuvant. Following injections, rat dam AB titers were confirmed via ELISA analysis of dam sera prior to breeding. Litters from MAR and control dams were culled to four males and four females at post-natal day 2 (PND2) and underwent a battery of behavioral tests at different time points to determine MAR influences on behavioral outcomes throughout the lifespan.

Communication Deficits in MAR Offspring. Data indicated that MAR offspring (N=48) produced significantly fewer bouts of isolation calls when temporarily separated from their mothers at PND12 compared to controls (N=40).

Decreased Social Play and Increased Repetitive Behavior. Rats exhibit prolonged periods of juvenile play that provide an important indicator of social development. Juvenile MAR offspring (N=24) exhibited less time engaged in play behavior compared to control animals (N=20) and that adult MAR offspring spend more time engaged in spontaneous self-grooming compared to control animals (N=20). In rodent models, self-grooming is a useful readout to assess repetitive behavior and is a task that involves proper coordination of complex neural circuits (89).

Impairments in Prosocial Behavior. A study to evaluate species-typical “prosocial behavior” exhibited by rats. Prosocial helping behavior can be assessed in rats using a task in which a rat learns to help a familiar rat escape from confinement by opening an escape door (90). The IDDRC Rodent Behavior Core (RBC) has found that both sexes of young and adult rats readily learn this helping behavior. Interestingly, both MAR-exposed and control rats were able to learn the test, although the MAR treated offspring deviated from the species-typical prosocial response as testing progressed.

Experimental Approach: MAR offspring from the antigen driven mouse model display reductions in species-typical mouse social behavior and pilot data from our antigen-driven rat model suggest similar reductions in species-typical rat social behavior. Subsequent studies will be planned to capitalize on the rich social repertoire of the rat to determine behavioral consequences following prenatal exposure to MAR ASD-specific antibodies if the administration of IV-anti-FcRn will rescue those behaviors.

Generation of Epitope-Specific AB. The study will be conducted in strict adherence to the recommendations and approval of the UC Davis IACUC Committee. Sprague Dawley rats will be purchased from a commercial vendor and housed in same-sex, same-strain pairs in a temperature and humidity-controlled vivarium on a 12 h light-dark cycle. Access to food and water will be ad libitum throughout. Methods established in the rat pilot studies described above, which established feasibility using antigens from the original model pattern of LDH-A+LDH-B+STIP1+CRMP1, will be followed. In rat dams, an antibody response will be induced to generate MAR specific antibodies by using the Multiple Antigenic Peptides (MAPs) system. MAR-specific peptides will be purchased (LifeTein, NJ) and conjugated to a 4-branch lysine backbone scaffold (LifeTein, Germany) to create a high molar ratio of peptide to core molecule and abrogates the need for carrier protein. This same strategy has been demonstrated in the described studies to successfully generate the AB of interest in sexually naïve female mice, and in our rat pilot studies. To confirm MAR specific antibody induction, sera will be collected and MAR antibody titers determined by ELISA. To generate AB in female Sprague Dawley rats prior to breeding, tolerance to the peptide epitopes of the MAR-specific autoantigens LDH-A+LDH-B+STIP1+CRMP1 will be broken. Antibody titers to these targets will be generated in the adult females prior to breeding. This will expose the embryos to AB generated in the dam throughout gestation with no inflammation.

Control treated females will receive an identical number and volume of immunizations consisting of Freund's adjuvant in saline. Following confirmation of AB production, dams will be paired with established breeder males using a 2:1 dam:sire breeding scheme. Breeding cohorts will be scheduled to accommodate offspring behavior studies and will include representatives from each treatment group. To achieve maximal statistical power based on our previous studies, at least 36 offspring (18 male, 18 female) per experimental treatment group will be produced for subsequent behavioral analyses.

Anti-FcRn protocol. Timed-pregnant females will be treated with a 5 mg/dose of anti-FcRn in saline IV on Days 10 and 15 of gestation. Control animals will receive 5 mg/dose of the wild-type control IgG. The effects of anti-FcRn in pilot studies demonstrated max reduction in MAR antibodies between 48-72 hrs in the pilot study. IgG does not begin to cross the placenta until Day 12 in rodents. Thus, the first injection will be performed on Day 10, to ensure the blocking antibody is in place when IgG begins to cross the placenta. The effect of the blocking antibody should last approximately 5 days thus a second dose will be administered on Day 15. Pups will be born beginning on Day 20-21.

Standard Behavioral Assessments. All behavioral studies will be carried out by the rat subcore of the IDDRC Rodent Behavior Core (RBC). Comprehensive, longitudinal behavioral studies will be carried out using RBC-established protocols (88). There will be 18 male and 18 female offspring per treatment group. Developmental milestones and neurological reflexes will be assessed using a standardized battery for rats, including pup isolation ultrasonic vocalization calls (USVs) on PND 4, 8, 12. On PND 21, the offspring will be weaned and housed same sex, two to a cage counterbalanced for treatment. The effects of prenatal MAR exposure will be characterized using a behavioral test battery that will began at PND 26 and conclude at PND 150. Tests will be conducted on separate days in an order designed to reduce the influence of sequential testing and will assess a range of behaviors at juvenile, adolescent and adult time points. Interactions with novel social partners (social dyads) will be recorded on PND 36, 55, 103 to further explore the social deficits described in the MAR pilot. In addition to duration measures, the frequency of positive social interactions initiated and received will be quantified to provide insight into the factors contributing to reduced social interactions in the MAR exposed rats. Appropriate control measures, including exploration (open field), sensorimotor gating (pre-pulse inhibition [PPI]), anxiety (elevated plus maze) and repetitive behaviors (quantified during dyads) will also be evaluated. As cognitive development has not been evaluated in the MAR rat model, evaluations of cognition from our existing test battery will include: (i) Contextual fear conditioning, (ii) Barnes/Morris water mazes, (iii) Novel Object (location, memory, temporal order), (iv) T-maze reversal learning (perseverative behaviors), and/or (V) touch screen tests (unique to our IDDRC RBC).

Expanded Characterization of Social Development. Behavioral assays designed to probe the sophisticated social repertoire of rats to further characterize the global reductions in social interaction observed in the MAR pilot will also be performed. (a) Isolation call classification—The RBC recently developed protocols for categorizing rat pup isolation USVs and will apply this approach to evaluate developmental trajectories of isolation call development beyond the simplistic automated assessment of call frequency. (b) Playback approach—As rats mature, USVs serve as situation-dependent affective signals that convey important communicative functions. Prosocial ultrasonic communication can be studied in a reliable and highly standardized manner by means of the 50-kHz USV radial maze playback paradigm described in and recently adapted by the Silverman Laboratory for use in the RBC. Radial maze playback paradigm will be used to determine if MAR offspring demonstrate species-typical responses to pro-social 50-kHz USVs following each social dyad. (c) Social motivation—A new test of prosocial motivation has been added that assess deficits in species-typical prosocial behavior. Pilot data from previous MAR cohorts suggests that MAR offspring do not show a species-typical response to familiar conspecifics in distress, which has been interpreted as an essential precursor to empathy. Pro-social behavior will be compared between adult MAR and control rats.

Statistical Analysis: Statistical analyses will be carried out by the BBRDC. From cross-sectional behavioral tests, age-dependent profiles of behavioral measurements among the five different groups of MAR treated rats and controls will be studied in both male and female. Group differences in behavioral measures will be determined using an analysis of variance (ANOVA) or multivariate regression analysis with important confounders (e.g., gender, body weight, lean mass, fat mass, etc.) as covariates. Where significant group differences are found, post-hoc analyses using the Tukey or Dunnet test to determine which two groups are specifically different from each other will be performed. For longitudinal data, generalized linear and latent mixed-effects model framework that accommodates traditional generalized linear models and classical latent variable models for multivariate data will be used, including exploratory and confirmatory factor analysis measurement models (that relate multiple correlated behavior test modules), and structural equations models. This framework provides a comprehensive assessment of behavioral characteristics associated with MAR ABs. All assumptions of the parametric statistical tests will be checked and, if they are not met, the data will either be transformed to meet assumptions, or a non-parametric test will be substituted. Since multiple outcomes will be measured from the same animal, statistical correction for multiple testing is warranted, such as Bonferroni correction or false discovery rate (FDR).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

| SEQ ID NO | SEQUENCE |
|---|---|
| 1 | CPPCPAPELLGGPSVFLFPPKPKDTLYITREPEVTCVVVDVSHEDPEVKFN |
| | WYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYP |
| | SDIAVEWESNGQPENNYKTTPPVLDSDGSFFLYSKLTVDKSRWQQGNVF |
| | SCSVMHEALKFHYTQKSLSLSPG |
| 2 | DKTHTCPPCPAPELLGGPSVFLFPPKPKDTLYITREPEVTCVVVDVSHEDP |
| | EVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCLV |
| | KGFYPSDIAVEWESNGQPENNYKTTPPVLDSDGSFFLYSKLTVDKSRWQ |
| | QGNVFSCSVMHEALKFHYTQKSLSLSPGK |
| 3 | DKTHTCPPCPAPELLGGPSVFLFPPKPKDTLYITREPEVTCVVVDVSHEDP |
| | EVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCLV |
| | KGFYPSDIAVEWESNGQPENNYKTTPPVLDSDGSFFLYSKLTVDKSRWQ |
| | QGNVFSCSVMHEALKFHYTQKSLSLSPG |

1. A method of reducing auto-reactive antibodies in a pregnant individual, an individual trying to become pregnant, an individual considering pregnancy, or an individual serving as a surrogate for a pregnancy, comprising administering an inhibitor of neonatal Fc receptor (FcRn) function to the pregnant individual, the individual trying to become pregnant, the individual considering pregnancy, or the individual serving as a surrogate for a pregnancy, thereby reducing the auto-reactive antibodies in the pregnant individual.

2. The method of claim 1, wherein the auto-reactive antibodies comprise antibodies that bind a central nervous system (CNS) target.

3. The method of claim 2, wherein an embryo or fetus expresses the CNS target.

4. The method of claim 1, wherein the auto-reactive antibodies bind to a target selected from the group consisting of lactate dehydrogenase A or B (LDH A,B), guanine deaminase (GDA), collapsin response mediator protein 1 or 2 (CRMP1,2)), stress-induced phosphoprotein 1 (STIP1), alpha subunit of the barbed-end actin binding protein Cap Z (CAPZA2), Y Box Binding Protein 1 (YBX1), eukaryotic translation and elongation factor 1A1 (EEF1A1), microtubule-associated protein Tau (MAPT), dihydropyrimidinase-like protein 2 (DPYSL2), dynamin 1-like protein (DNM1L), radixin (RDX), moesin (MSN), non-specific enolase (NSE), Caspr2, KCNAB2, KCNAB1, endothelial integrin ligand (EDIL3), IVD, brain specific sultatransferase (SULT4A1), TNIP2, retinoic acid induced 16 (RAI16), GDP D5, ezrin (EZR), and any combination thereof.

5. The method of claim 4, wherein the auto-reactive antibodies bind to a target selected from the group consisting of lactate dehydrogenase A (LDHA), lactate dehydrogenase B (LDHB), collapsin response mediator protein 1 (CRMP1), stress-induced phosphoprotein 1 (STIP1), and any combination thereof.

6. The method of claim 4, wherein the auto-reactive antibodies bind to a target selected from the group consisting of lactate dehydrogenase collapsin response mediator protein 1 (CRMP1), stress-induced phosphoprotein 1 (STIP1), and both collapsin response mediator protein 1 (CRMP1), stress-induced phosphoprotein 1 (STIP1).

7-103. (canceled)

104. A method of reducing auto-reactive antibodies contacting an embryo or fetus in a pregnant individual, an individual trying to become, an individual considering pregnancy or an individual serving as a surrogate for the pregnancy, comprising administering, to the pregnant individual, the individual trying to become pregnant, the individual considering pregnancy or the individual serving as a surrogate for the pregnancy, an inhibitor of neonatal Fc receptor (FcRn) function.
